(19) United States
(12) Patent Application Publication
(10) Pub. No.: US 2021/0236838 A1
(43) Pub. Date: Aug. 5, 2021

(54) PHOTOBIOMODULATION THERAPY SYSTEMS AND METHODS

(71) Applicant: Joovv, Inc., San Clemente, CA (US)

(72) Inventors: Stuart Pfiffner, San Clemente, CA (US); Greg Pfiffner, San Clemente, CA (US)

(21) Appl. No.: 17/236,950

(22) Filed: Apr. 21, 2021

Related U.S. Application Data
(63) Continuation-in-part of application No. 16/167,385, filed on Oct. 22, 2018, now Pat. No. 11,033,752.

(51) Int. Cl. A61N 5/06

(52) U.S. Cl. CPC A61N 5/06; A61N 2005/064; A61N 2005/0632

(57) ABSTRACT

A light therapy device mounting kit may include a first spacer comprising a first flat base, a first leg protruding away from the first flat base, and a second leg spaced from the first leg, wherein the second leg also protrudes away from the first flat base. In some embodiments, each of the first leg and the second leg is configured to slideably engage a cavity located within a light therapy device. Each leg may be configured to slideably engage one cavity located in two different light therapy devices. The light therapy device mounting kit may include vertical spacer(s) comprising two legs, horizontal spacer(s) comprising two legs, and square spacer(s) comprising four legs.

### CROSS-REFERENCE TO RELATED APPLICATIONS

The entire contents of the following application are incorporated by reference herein: U.S. Provisional Patent Application No. 62/460,113; filed Feb. 17, 2017; and entitled THERAPEUTIC LIGHT SOURCE AND HANGING APPARATUS.

The entire contents of the following application are incorporated by reference herein: U.S. patent application Ser. No. 15/616,028; filed Jun. 7, 2017; and entitled THERAPEUTIC LIGHT SOURCE AND HANGING APPARATUS (now U.S. Pat. No. 10,828,505).

The entire contents of the following application are incorporated by reference herein: PCT Patent Application No. PCT/US18/18288; filed Mar. 1, 2018; and entitled THERAPEUTIC LIGHT SOURCE AND MOUNTING APPARATUS.

The entire contents of the following application are incorporated by reference herein: U.S. patent application Ser. No. 16/167,385; filed Oct. 22, 2018; and entitled PHOTOBIOMODULATION THERAPY SYSTEMS AND METHODS.

The entire contents of the following application are incorporated by reference herein: U.S. patent application Ser. No. 16/227,289; filed Dec. 20, 2018; and entitled PHOTOBIOMODULATION THERAPY SYSTEMS AND METHODS (now U.S. Pat. No. 10,478,635).

The entire contents of the following application are incorporated by reference herein: U.S. Provisional Patent Application No. 62/863,247; filed Jun. 18, 2019; and entitled PHOTOBIOMODULATION THERAPY SYSTEMS AND METHODS.

The entire contents of the following application are incorporated by reference herein: U.S. Provisional Patent Application No. 62/872,835; filed Jul. 11, 2019; and entitled PHOTOBIOMODULATION THERAPY SYSTEMS AND METHODS.

The entire contents of the following application are incorporated by reference herein: U.S. Patent Application No. 16/584,784; filed September 26, 2019; and entitled PHOTOBIOMODULATION THERAPY SYSTEMS AND METHODS (now U.S. Pat. No. 10,639,495).

The entire contents of the following application are incorporated by reference herein: U.S. patent application Ser. No. 16/598,033; filed Oct. 10, 2019; and entitled PHOTOBIOMODULATION THERAPY SYSTEMS AND METHODS.

The entire contents of the following application are incorporated by reference herein: PCT Patent Application No. PCT/US2019/057292; filed Oct. 21, 2019; and entitled PHOTOBIOMODULATION THERAPY SYSTEMS AND METHODS.

The entire contents of the following application are incorporated by reference herein: PCT Patent Application No. PCT/US2019/059845; filed Nov. 5, 2019; and entitled PHOTOBIOMODULATION THERAPY SYSTEMS AND METHODS.

The entire contents of the following application are incorporated by reference herein: U.S. Non-Provisional patent application Ser. No. 16/904,243; filed Jun. 17, 2020; and entitled PHOTOBIOMODULATION THERAPY SYSTEMS AND METHODS.

The entire contents of the following application are incorporated by reference herein: PCT Patent Application No. PCT/US2020/038187; filed Jun. 17, 2020; and entitled PHOTOBIOMODULATION THERAPY SYSTEMS AND METHODS.

The entire contents of the following application are incorporated by reference herein: U.S. Non-Provisional patent application Ser. No. 17/027,338; filed Sep. 21, 2020; and entitled PHOTOBIOMODULATION THERAPY DEVICE ACCESSORIES.

The entire contents of the following application are incorporated by reference herein: U.S. Non-Provisional patent application Ser. No. 17/027,472; filed Sep. 21, 2020; and entitled PHOTOBIOMODULATION THERAPY SYSTEMS AND METHODS.

### BACKGROUND

### Field

Various embodiments disclosed herein relate to photobiomodulation therapy systems and methods.

### Background

Photobiomodulation therapy (or light therapy) is a therapeutic technique that uses low-level wavelengths of light to improve health and treat a variety of health conditions, including skin issues, such as wrinkles, scars, and persistent wounds, among many other conditions. Photobiomodulation therapy uses non-ionizing light sources, including lasers, light emitting diodes, and/or broadband light, in the visible (400-700 nm) and infrared (700-1100 nm) electromagnetic spectrum. Photobiomodulation is a nonthermal process involving endogenous chromophores eliciting photophysical (i.e., linear and nonlinear) and photochemical events at various biological scales. Similar to how plants use sunlight to heal and grow, humans and animals are able to harness these wavelengths of light and turn them into cellular energy. This treatment stimulates the body's natural healing processes.

Light therapy devices are sometimes available with a stand, mount, hanging mechanism, or similar device to support the light therapy device(s). Some examples include height-adjustable hanging mechanisms. There is a need for a broader range of supportive accessories for light therapy devices.

### SUMMARY

The disclosure includes a variety of embodiments whereby two or more light devices are electrically and/or mechanically coupled together to form a modular photobiomodulation therapy system. The term “photobiomodulation” shall be used interchangeably with the term “light”. For the purposes of this disclosure, “photobiomodulation” and “light” shall include wavelengths of light in the red, blue, green, and near infrared spectrums.

In some embodiments, the photobiomodulation therapy system includes a first light device and a second light device arranged and configured to be coupled to the first light device. The first light device may include a first housing and a first plurality of lights arranged and configured to emit at least one of red light, blue light, green light, and/or near infrared light. Additionally, the second light device may include a second housing and a second plurality of lights arranged and configured to emit at least one of red light, blue light, green light, and/or near infrared light. In some embodiments, the first light device is arranged and configured to be electrically coupled to the second light device.

In many embodiments, the first light device comprises a first power management system electrically coupled to the first plurality of lights, and the second light device comprises a second power management system electrically coupled to the second plurality of lights. Embodiments may thereby include a first power cord arranged and configured to be electrically coupled to the first power management system and a first power outlet. Additionally, embodiments may include a second power cord arranged and configured to be electrically coupled to the first power management system and the second power management system. This may allow the first light device and the second light device to be electrically paired together in series.

The photobiomodulation therapy system can be expanded by adding a third light device arranged and configured to be coupled to at least one of the first light device and the second light device, and a fourth light device arranged and configured to be coupled to at least one of the first light device, second light device, and third light device. The third light device may include a third housing, a third plurality of lights arranged and configured to emit at least one of red light, blue light, green light, and/or near infrared light, and a third power management system electrically coupled to the third plurality of lights. Accordingly, the fourth light device may include a fourth housing, a fourth plurality of lights arranged and configured to emit at least one of red light, blue light, green light, and/or near infrared light, and a fourth power management system electrically coupled to the fourth plurality of lights. Embodiments may thereby include a third power cord arranged and configured to be electrically coupled to the third power management system and a second power outlet. Additionally, embodiments may include a fourth power cord arranged and configured to be electrically coupled to the third power management system and the fourth power management system. This may allow the third light device and the fourth light device to be electrically coupled in series, and these together are in parallel with the first light device and the second light device.

Embodiments may also include a third power cord that can be arranged and configured to be electrically coupled to the second power management system and a third power management system. Additionally, embodiments may include a fourth power cord that may also be arranged and configured to be electrically coupled to the third power management system and the fourth power management system. This may allow the first light device, second light device, third light device, and fourth light device to all be electrically coupled in series.

The photobiomodulation therapy system can further be expanded to include a fifth light device arranged and configured to be coupled to at least one of the first light device, second light device, third light device, and fourth light device, and a sixth light device arranged and configured to be coupled to at least one of the first light device, second light device, third light device, fourth light device, and fifth light device. The fifth light device may include a fifth housing, a fifth plurality of lights arranged and configured to emit at least one of red light, blue light, green light, and/or near infrared light, and a fifth power management system electrically coupled to the fifth plurality of lights. Likewise, the sixth light device may include a sixth housing, a sixth plurality of lights arranged and configured to emit at least one of red light, blue light, green light, and/or near infrared light, and a sixth power management system electrically coupled to the sixth plurality of lights. Such embodiments may thereby include a fourth power cord electrically coupled to the second power outlet. Additionally, embodiments may include a fifth power cord arranged and configured to be electrically coupled to the fourth power management system and the fifth power management system, and a sixth power cord arranged and configured to be electrically coupled to the fifth power management system and the sixth power management system. This may allow the fourth light device, fifth light device, and sixth light device to be electrically coupled in series, and paired together in parallel with the first, second, and third light devices.

Alternatively, embodiments may be configured whereby all of the light devices are coupled together in series. In this regard, embodiments may thereby include the fourth power cord arranged and configured to be electrically coupled to the third power management system and the fourth power management system. Accordingly, embodiments may include a fifth power cord arranged and configured to be electrically coupled to the fourth power management system and a fifth power management system, and a sixth power cord arranged and configured to be electrically coupled to the fifth power management system and the sixth power management system. As previously stated, this may allow the first light device, second light device, third light device, fourth light device, fifth light device, and sixth light device to all be electrically coupled in series.

In addition to electrical coupling, two or more light devices may be mechanically coupled together to form a variety of sizes and configurations. In some embodiments, the system includes a first aperture located on a bottom side of the first housing, and a second aperture located on the bottom side of the first housing. Additionally, embodiments may include a first attachment member extending from a top surface of the second housing. The first attachment member may be arranged and configured to be detachably coupled to the first aperture to thereby couple the first housing to the second housing. Accordingly, embodiments may include a second attachment member extending from the top surface of the second housing. The second attachment member may be arranged and configured to be detachably coupled to the second aperture to thereby couple the first housing to the second housing. Mechanically coupling the first light device and the second light device in this way achieves a top-to-bottom orientation.

Alternatively, some embodiments may be mechanically coupled in a side-by-side orientation. In some embodiments, the system includes a first bracket arranged and configured to be detachably coupled to a back side of the first housing and a back side of the second housing to thereby detachably couple the first housing to the second housing. Additionally, some embodiments include a second bracket arranged and configured to be detachably coupled to the back side of the first housing and the back side of the second housing to detachably couple the first housing to the second housing.

In some embodiments, the system includes a door stand to thereby mount the light therapy system to a door. In this regard, some embodiments include a first attachment member extending from a top surface of the first housing. The first attachment member may be arranged and configured to be detachably coupled to a first door stand. Additionally, some embodiments include a second attachment member extending from the top surface of the first housing. The second attachment member may be arranged and configured to be detachably coupled to the first door stand. Accordingly, some embodiments include a third attachment member extending from a top surface of the second housing. The third attachment member may be arranged and configured to be detachably coupled to a second door stand. Additionally, some embodiments include a fourth attachment member extending from the top surface of the second housing. Likewise, the fourth attachment member may be arranged and configured to be detachably coupled to the second door stand.

From a mechanical coupling perspective, the light therapy system can be expanded by adding a third light device arranged and configured to be detachably coupled to at least one of the first light device and the second light device, and a fourth light device arranged and configured to be detachably coupled to at least one of the first light device, second light device, and third light device. The third light device may include a third housing and a third plurality of lights arranged and configured to emit at least one of red light, blue light, green light, and/or near infrared light. Accordingly, the fourth light device may include a fourth housing and a fourth plurality of lights arranged and configured to emit at least one of red light, blue light, green light, and/or near infrared light. Some embodiments include a third bracket arranged and configured to be detachably coupled to a back side of the third housing and a back side of the fourth housing to thereby detachably couple the third housing to the fourth housing. Additionally, some embodiments include a fourth bracket arranged and configured to be detachably coupled to the back side of the third housing and the back side of the fourth housing to thereby detachably couple the third housing to the fourth housing.

Even still, in some embodiments, the system includes a first aperture and a second aperture both located on a bottom side of the first housing, and a third aperture and a fourth aperture both located on a bottom side of the second housing. Some embodiments may thereby include a fifth attachment member extending from a top surface of the third housing. The fifth attachment member may be arranged and configured to be detachably coupled to the first aperture. Accordingly, some embodiments include a sixth attachment member extending from the top surface of the third housing. The sixth attachment member may be arranged and configured to be detachably coupled to the second aperture. Some embodiments may also include a seventh attachment member extending from a top surface of the fourth housing. The seventh attachment member may be arranged and configured to be detachably coupled to the third aperture. Even still, some embodiments may include an eighth attachment member extending from the top surface of the fourth housing. The eighth attachment member may be arranged and configured to be detachably coupled to the fourth aperture.

Some system embodiments disclosed herein include a mobile stand. In this regard, the first housing and the second housing may include a top side, a bottom side, a front side, and a back side. The system may further include a third light device arranged and configured to be detachably coupled to at least one of the first light device and the second light device, and a fourth light device arranged and configured to be detachably coupled to at least one of the first light device, the second light device, and the third light device. In some embodiments, the third light device includes a third housing and a third plurality of lights arranged and configured to emit at least one of red light, blue light, green light, and/or near infrared light. Accordingly, in some embodiments, the fourth light device includes a fourth housing and a fourth plurality of lights arranged and configured to emit at least one of red light, blue light, green light, and/or near infrared light. Additionally, some embodiments include a first bracket arranged and configured to be detachably coupled to a back side of the first housing and a back side of the second housing to thereby detachably couple the first housing to the second housing. Similarly, some embodiments include a second bracket arranged and configured to be detachably coupled to a back side of the third housing and a back side of the fourth housing to thereby detachably couple the third housing to the fourth housing. Some embodiments include a quad bracket arranged and configured to be detachably coupled to the back side of the first housing, the back side of the second housing, the back side of the third housing, and the back side of the fourth housing to thereby detachably couple the first housing, second housing, third housing, and fourth housing together. The quad bracket may be arranged and configured to be detachably coupled to a mobile stand.

In some embodiments, the first housing and the second housing may include a top side, a bottom side, a front side, and a back side. The system may further include a third light device arranged and configured to be detachably coupled to at least one of the first light device and the second light device, a fourth light device arranged and configured to be detachably coupled to at least one of the first light device, the second light device, and the third light device, a fifth light device arranged and configured to be detachably coupled to at least one of the first light device, the second light device, the third light device, and the fourth light device, and a sixth light device arranged and configured to be detachably coupled to at least one of the first light device, the second light device, the third light device, the fourth light device, and the fifth light device. In some embodiments, the third light device includes a third housing and a third plurality of lights arranged and configured to emit at least one of red light, blue light, green light, and/or near infrared light. Accordingly, in some embodiments the fourth light device includes a fourth housing and a fourth plurality of lights arranged and configured to emit at least one of red light, blue light, green light, and/or near infrared light. Similarly, in some embodiments the fifth light device includes a fifth housing and a fifth plurality of lights arranged and configured to emit at least one of red light, blue light, green light, and/or near infrared light. Finally, some embodiments include the sixth light device, which includes a sixth housing and a sixth plurality of lights arranged and configured to emit at least one of red light, blue light, green light, and/or near infrared light. Additionally, some embodiments include a first bracket arranged and configured to be detachably coupled to a back side of the first housing, a back side of the second housing, and a back side of the fifth housing to thereby detachably couple the first light device, second light device, and fifth light device together. Accordingly, some embodiments include a second bracket arranged and configured to be detachably coupled to a back side of the third housing, a back side of the fourth housing, and a back side of the sixth housing to thereby detachably couple the third light device, fourth light device, and sixth light device together. Additionally, some embodiments include a large bracket arranged and configured to be detachably coupled to the back side of the first housing, the back side of the second housing, the back side of the third housing, the back side of the fourth housing, the back side of the fifth housing, and the back side of the sixth housing to thereby detachably couple the first light device, second light device, third light device, fourth light device, fifth light device, and sixth light device together. In some embodiments, the large bracket is arranged and configured to be detachably coupled to a mobile stand.

In some embodiments, the system further includes the mobile stand having at least one vertical arm arranged and configured to couple to the large bracket. Additionally, in some embodiments, the mobile stand includes at least one horizontal leg arranged and configured to contact a floor surface.

In some embodiments, the first light device is a first modular light device and the second light device is a second modular light device whereby each of the first and second modular light devices are arranged and configured to be mechanically coupled in at least one of a side-by-side orientation and a top-to-bottom orientation. Furthermore, in some embodiments, the system includes a third modular light device and a fourth modular light device each arranged and configured to be mechanically coupled to at least one of the first, second, third, and fourth modular light devices in at least one of a side-by-side orientation and a top-to-bottom orientation.

The disclosure also includes a light therapy system, comprising a first light therapy device comprising a first rigid housing and a first plurality of lights configured to emit at least one of red light and near infrared light; and a second light therapy device comprising a second rigid housing and a second plurality of lights configured to emit at least one of red light and near infrared light, wherein the first rigid housing is configured to be detachably coupled to the second rigid housing.

In some embodiments. the first light therapy device and the second light therapy device are configured to be detachably coupled in a side-by-side orientation. In some embodiments, the first light therapy device and the second light therapy device are configured to be detachably coupled in a top-to-bottom orientation.

In some embodiments, the light therapy system further comprises a coupling system coupled to a bottom surface of the first rigid housing and a top surface of the second rigid housing, wherein the coupling system is configured to detachably couple the first rigid housing to the second rigid housing. In some embodiments, the first light therapy device comprises a first aperture located on the bottom surface of the first rigid housing, and a second aperture located on the bottom surface of the first rigid housing. In some embodiments, the coupling system comprises a first post extending from the top surface of the second rigid housing, and a second post extending from the top surface of the second rigid housing, the first post being configured to detachably couple to the first aperture, and the second post being configured to detachably couple to the second aperture.

In some embodiments, the light therapy system further comprises a first bracket configured to couple to both a back surface of the first rigid housing and a back surface of the second rigid housing to thereby couple the first rigid housing to the second rigid housing. Additionally, in some embodiments, the light therapy system further comprises a second bracket configured to couple to both the back surface of the first rigid housing and the back surface of the second rigid housing to thereby couple the first rigid housing to the second rigid housing.

Some embodiments further comprise: a third light therapy device comprising a third rigid housing and a third plurality of lights configured to emit at least one of red light and near infrared light; and a fourth light therapy device comprising a fourth rigid housing and a fourth plurality of lights configured to emit at least one of red light and near infrared light, wherein the third light therapy device and the fourth light therapy device are each configured to be detachably coupled to at least one of the first light therapy device and the second light therapy device.

In some embodiments, the first light therapy device comprises a first aperture located on a bottom surface of the first rigid housing, and the second light therapy device comprises a second aperture located on a bottom surface of the second rigid housing, wherein the third light therapy device comprises a first post extending from a top surface of the third rigid housing, the first post configured to detachably couple to the first aperture, and wherein the fourth light therapy device comprises a second post extending from a top surface of the fourth rigid housing, the second post configured to detachably couple to the second aperture.

In some embodiments, the light therapy system further comprises a first bracket configured to detachably couple to the first rigid housing and the second rigid housing, wherein a first portion of the first bracket is configured to couple to a back surface of the first rigid housing, and a second portion of the first bracket is configured to couple to a back surface of the second rigid housing; and a second bracket configured to detachably couple to the third rigid housing and the fourth rigid housing, wherein a first portion of the second bracket is configured to couple to a back surface of the third rigid housing, and a second portion of the second bracket is configured to couple to a back surface of the fourth rigid housing.

In some embodiments, the light therapy system further comprises a quad bracket configured to be detachably coupled to a back surface of the first rigid housing, a back surface of the second rigid housing, a back surface of the third rigid housing, and a back surface of the fourth rigid housing to thereby couple the first rigid housing, the second rigid housing, the third rigid housing and the fourth rigid housing together. In some embodiments, the light therapy system further comprises a mobile stand having at least one vertical arm configured to couple to the quad bracket.

In some embodiments, the mobile stand comprises at least one horizontal leg coupled to the at least one vertical arm, wherein the at least one horizontal leg is configured to stabilize the mobile stand; and a plurality of casters extending from the at least one horizontal leg, the plurality of casters configured to contact a ground surface and thereby enable the mobile stand to roll along the ground surface.

In some embodiments, the light therapy system further comprises an attachment system configured to couple to a top surface of the first rigid housing, wherein the attachment system is configured to detachably couple the first rigid housing and the second rigid housing to at least one of a door stand and a wall stand.

In some embodiments, the attachment system comprises: a first post extending from the top surface of the first rigid housing; a second post extending from the top surface of the first rigid housing, the second post spaced from the first post; and a cable extending from the first post to the second post, the cable configured to couple to at least one of the door stand and the wall stand.

In some embodiments, the light therapy system further comprises an attachment system configured to couple to a top surface of the first rigid housing and a top surface of the second rigid housing, wherein the attachment system is configured to detachably couple the first rigid housing and the second rigid housing to at least one of a door stand and a wall stand, wherein the attachment system further comprises: a first post extending from the top surface of the first rigid housing; a second post extending from the top surface of the first rigid housing, the second post spaced from the first post; a first cable extending from the first post to the second post, the first cable configured to couple to at least one of the door stand and the wall stand; a third post extending from the top surface of the second rigid housing; a fourth post extending from the top surface of the second rigid housing, the fourth post spaced from the third post; a second cable extending from the third post to the fourth post, the second cable configured to couple to at least one of the door stand and the wall stand.

In some embodiments, wherein the first light therapy device is configured to be electrically coupled to the second light therapy device.

In some embodiments, a first portion of the first plurality of lights is configured to emit red light and a first portion of the second plurality of lights is configured to emit red light. In some embodiments, a second portion of the first plurality of lights is configured to emit near infrared light and a second portion of the second plurality of lights is configured to emit near infrared light.

In some embodiments, the first plurality of lights are at least partially encompassed within the first rigid housing and thereby protrude through the first rigid housing, and wherein the second plurality of lights are at least partially encompassed within the second rigid housing and thereby protrude through the second rigid housing.

The disclosure also include a photobiomodulation therapy system, comprising: a first light device comprising a first rigid housing, a first printed circuit board (PCB) located within the first rigid housing, and a first plurality of light sources electrically coupled to the first PCB, wherein the first plurality of light sources are at least partially encompassed within the first rigid housing and thereby protrude through the first rigid housing, and wherein a first portion of the first plurality of light sources emits near infrared light and a second portion of the first plurality of light sources emits red light; a second light device arranged and configured to be detachably coupled to the first light device, the second light device having a second rigid housing, a second printed circuit board (PCB) located within the second rigid housing, and a second plurality of light sources electrically coupled to the second PCB, wherein the second plurality of light sources are at least partially encompassed within the second rigid housing and thereby protrude through the second rigid housing, and wherein a first portion of the second plurality of light sources emits near infrared light and a second portion of the second plurality of light sources emits red light; a first aperture located on a bottom side of the first rigid housing; a second aperture located on the bottom side of the first rigid housing; a first attachment member extending from a top surface of the second rigid housing, wherein the first attachment member is arranged and configured to be detachably coupled to the first aperture to thereby couple the first rigid housing to the second rigid housing; and a second attachment member extending from the top surface of the second rigid housing, wherein the second attachment member is arranged and configured to be detachably coupled to the second aperture to thereby couple the first rigid housing to the second rigid housing.

In some embodiments, the first light device is arranged and configured to be electrically coupled to the second light device. In some embodiments, the first light device comprises a first power management system electrically coupled to the first plurality of light sources, and the second light device comprises a second power management system electrically coupled to the second plurality of light sources.

In some embodiments, the system further comprises: a first power cord arranged and configured to be electrically coupled to the first power management system and a first power outlet; and a second power cord arranged and configured to be electrically coupled to the first power management system and the second power management system.

In some embodiments, the system further comprises: a third power cord arranged and configured to be electrically coupled to a third power management system and a second power outlet; and a fourth power cord arranged and configured to be electrically coupled to the third power management system and a fourth power management system.

In some embodiments, the system further comprises: a fifth light device arranged and configured to be detachably coupled to at least one of the first light device, the second light device, the third light device, and the fourth light device, the fifth light device comprising a fifth rigid housing, a fifth printed circuit board (PCB) located within the fifth rigid housing, a fifth plurality of light sources electrically coupled to the fifth PCB, wherein the fifth plurality of light sources are at least partially encompassed within the fifth rigid housing and thereby protrude through the fifth rigid housing, and wherein a first portion of the fifth plurality of light sources emits near infrared light and a second portion of the fifth plurality of light sources emits red light, and a fifth power management system electrically coupled to the fifth plurality of light sources; and a sixth light device arranged and configured to be detachably coupled to at least one of the first light device, the second light device, the third light device, the fourth light device, and the fifth light device, the sixth light device comprising a sixth rigid housing, a sixth printed circuit board (PCB) located within the sixth rigid housing, a sixth plurality of light sources electrically coupled to the sixth PCB, wherein the sixth plurality of light sources are at least partially encompassed within the sixth rigid housing and thereby protrude through the sixth rigid housing, and wherein a first portion of the sixth plurality of light sources emits near infrared light and a second portion of the sixth plurality of light sources emits red light, and a sixth power management system electrically coupled to the sixth plurality of light sources.

In some embodiments, the system further comprises: a third power cord arranged and configured to be electrically coupled to a second power management system and the a third power management system; a fourth power cord arranged and configured to be electrically coupled to a fourth power management system and a second power outlet; a fifth power cord arranged and configured to be electrically coupled to the fourth power management system and the fifth power management system; and a sixth power cord arranged and configured to be electrically coupled to the fifth power management system and the sixth power management system.

In some embodiments, the system further comprises: a third power cord arranged and configured to be electrically coupled to a second power management system and a third power management system; and a fourth power cord arranged and configured to be electrically coupled to the third power management system and a fourth power management system.

In some embodiments, the system further comprises: a fifth light device arranged and configured to be detachably coupled to at least one of the first light device, the second light device, the third light device, and the fourth light device, the fifth light device comprising a fifth rigid housing, a fifth printed circuit board (PCB) located within the fifth rigid housing, a fifth plurality of light sources electrically coupled to the fifth PCB, wherein the fifth plurality of light sources are at least partially encompassed within the fifth rigid housing and thereby protrude through the fifth rigid housing, and wherein a first portion of the fifth plurality of light sources emits near infrared light and a second portion of the fifth plurality of light sources emits red light, and a fifth power management system electrically coupled to the fifth plurality of light sources; a sixth light device arranged and configured to be detachably coupled to at least one of the first light device, the second light device, the third light device, the fourth light device, and the fifth light device, the sixth light device comprising a sixth rigid housing, a sixth printed circuit board (PCB) located within the sixth rigid housing, a sixth plurality of light sources electrically coupled to the sixth PCB, wherein the sixth plurality of light sources are at least partially encompassed within the sixth rigid housing and thereby protrude through the sixth rigid housing, and wherein a first portion of the sixth plurality of light sources emits near infrared light and a second portion of the sixth plurality of light sources emits red light, and a sixth power management system electrically coupled to the sixth plurality of light sources; a fifth power cord arranged and configured to be electrically coupled to the fourth power management system and the fifth power management system; and a sixth power cord arranged and configured to be electrically coupled to the fifth power management system and the sixth power management system.

In some embodiments, the system further comprises: a first bracket arranged and configured to be detachably coupled to a back side of the first rigid housing and a back side of the second rigid housing to thereby detachably couple the first rigid housing to the second rigid housing; a second bracket arranged and configured to be detachably coupled to the back side of the first rigid housing and the back side of the second rigid housing to thereby detachably couple the first rigid housing to the second rigid housing; a first attachment member extending from a top surface of the first rigid housing, wherein the first attachment member is arranged and configured to be detachably coupled to a first door stand; a second attachment member extending from the top surface of the first rigid housing, wherein the second attachment member is arranged and configured to be detachably coupled to the first door stand; a third attachment member extending from a top surface of the second rigid housing, wherein the third attachment member is arranged and configured to be detachably coupled to a second door stand; and a fourth attachment member extending from the top surface of the second rigid housing, wherein the fourth attachment member is arranged and configured to be detachably coupled to the second door stand.

In some embodiments, the system further comprises: a third light device arranged and configured to be detachably coupled to at least one of the first light device and the second light device, the third light device comprising a third rigid housing, a third printed circuit board (PCB) located within the third rigid housing, and a third plurality of light sources electrically coupled to the third PCB, wherein the third plurality of light sources are at least partially encompassed within the third rigid housing and thereby protrude through the third rigid housing, and wherein a first portion of the third plurality of light sources emits near infrared light and a second portion of the third plurality of light sources emits red light; a fourth light device arranged and configured to be detachably coupled to at least one of the first light device, the second light device, and the third light device, the fourth light device comprising a fourth rigid housing, a fourth printed circuit board (PCB) located within the fourth rigid housing, and a fourth plurality of light sources electrically coupled to the fourth PCB, wherein the fourth plurality of light sources are at least partially encompassed within the fourth rigid housing and thereby protrude through the fourth rigid housing, and wherein a first portion of the fourth plurality of light sources emits near infrared light and a second portion of the fourth plurality of light sources emits red light; a third bracket arranged and configured to be detachably coupled to a back side of the third rigid housing and a back side of the fourth rigid housing to thereby detachably couple the third rigid housing to the fourth rigid housing; and a fourth bracket arranged and configured to be detachably coupled to the back side of the third rigid housing and the back side of the fourth rigid housing to thereby detachably couple the third rigid housing to the fourth rigid housing.

In some embodiments, the system further comprises: a first aperture and a second aperture both located on a bottom side of the first rigid housing; a third aperture and a fourth aperture both located on a bottom side of the second rigid housing; a fifth attachment member extending from a top surface of the third rigid housing, wherein the fifth attachment member is arranged and configured to be detachably coupled to the first aperture; a sixth attachment member extending from the top surface of the third rigid housing, wherein the sixth attachment member is arranged and configured to be detachably coupled to the second aperture; a seventh attachment member extending from a top surface of the fourth rigid housing, wherein the seventh attachment member is arranged and configured to be detachably coupled to the third aperture; and an eighth attachment member extending from the top surface of the fourth rigid housing, wherein the eighth attachment member is arranged and configured to be detachably coupled to the fourth aperture.

In some embodiments, each of the first rigid housing and the second rigid housing comprises a top side, a bottom side, a front side, and a back side, the system further comprising: a first bracket arranged and configured to be detachably coupled to a back side of the first rigid housing and a back side of the second rigid housing to thereby detachably couple the first rigid housing to the second rigid housing; and a second bracket arranged and configured to be detachably coupled to a back side of the third rigid housing and a back side of the fourth rigid housing to thereby detachably couple the third rigid housing to the fourth rigid housing.

In some embodiments, each of the first rigid housing and the second rigid housing comprises a top side, a bottom side, a front side, and a back side, the system further comprising: a third light device arranged and configured to be detachably coupled to at least one of the first light device and the second light device, the third light device comprising a third rigid housing, a third printed circuit board (PCB) located within the third rigid housing, and a third plurality of light sources electrically coupled to the third PCB, wherein the third plurality of light sources are at least partially encompassed within the third rigid housing and thereby protrude through the third rigid housing, and wherein a first portion of the third plurality of light sources emits near infrared light and a second portion of the third plurality of light sources emits red light; a fourth light device arranged and configured to be detachably coupled to at least one of the first light device, the second light device, and the third light device, the fourth light device comprising a fourth rigid housing, a fourth printed circuit board (PCB) located within the fourth rigid housing, and a fourth plurality of light sources electrically coupled to the fourth PCB, wherein the fourth plurality of light sources are at least partially encompassed within the fourth rigid housing and thereby protrude through the fourth rigid housing, and wherein a first portion of the fourth plurality of light sources emits near infrared light and a second portion of the fourth plurality of light sources emits red light; a fifth light device arranged and configured to be detachably coupled to at least one of the first light device, the second light device, the third light device, and the fourth light device, the fifth light device comprising a fifth rigid housing, a fifth printed circuit board (PCB) located within the fifth rigid housing, and a fifth plurality of light sources electrically coupled to the fifth PCB, wherein the fifth plurality of light sources are at least partially encompassed within the fifth rigid housing and thereby protrude through the fifth rigid housing, and wherein a first portion of the fifth plurality of light sources emits near infrared light and a second portion of the fifth plurality of light sources emits red light; a sixth light device arranged and configured to be detachably coupled to at least one of the first light device, the second light device, the third light device, the fourth light device, and the fifth light device, the sixth light device comprising a sixth rigid housing, a sixth printed circuit board (PCB) located within the sixth rigid housing, and a sixth plurality of light sources electrically coupled to the sixth PCB, wherein the sixth plurality of light sources are at least partially encompassed within the sixth rigid housing and thereby protrude through the sixth rigid housing, and wherein a first portion of the sixth plurality of light sources emits near infrared light and a second portion of the sixth plurality of light sources emits red light; a first bracket arranged and configured to be detachably coupled to a back side of the first rigid housing, a back side of the second rigid housing, and a back side of the fifth rigid housing to thereby detachably couple the first light device, second light device, and fifth light device together; a second bracket arranged and configured to be detachably coupled to a back side of the third rigid housing, a back side of the fourth rigid housing, and a back side of the sixth rigid housing to thereby detachably couple the third light device, fourth light device, and sixth light device together; and a large bracket arranged and configured to be detachably coupled to the back side of the first rigid housing, the back side of the second rigid housing, the back side of the third rigid housing, the back side of the fourth rigid housing, the back side of the fifth rigid housing, and the back side of the sixth rigid housing to thereby detachably couple the first light device, second light device, third light device, fourth light device, fifth light device, and sixth light device together, wherein the large bracket is arranged and configured to be detachably coupled to a mobile stand.

In some embodiments, the first light device is a first modular light device and the second light device is a second modular light device, the system further comprising a third modular light device and a fourth modular light device, whereby each of the first, second, third, and fourth modular light devices are arranged and configured to be mechanically coupled to at least one of the first, second, third, and fourth modular light devices in at least one of a side-by-side orientation and a top-to-bottom orientation.

The disclosure also includes a photobiomodulation therapy system, comprising: a first light device comprising a first rigid housing, a first printed circuit board (PCB) located within the first rigid housing, and a first plurality of light sources electrically coupled to the first PCB, wherein the first plurality of light sources are at least partially encompassed within the first rigid housing and thereby protrude through the first rigid housing, and wherein a first portion of the first plurality of light sources emits near infrared light and a second portion of the first plurality of light sources emits red light; a second light device arranged and configured to be detachably coupled to the first light device, the second light device having a second rigid housing, a second printed circuit board (PCB) located within the second rigid housing, and a second plurality of light sources electrically coupled to the second PCB, wherein the second plurality of light sources are at least partially encompassed within the second rigid housing and thereby protrude through the second rigid housing, and wherein a first portion of the second plurality of light sources emits near infrared light and a second portion of the second plurality of light sources emits red light; a third light device arranged and configured to be detachably coupled to at least one of the first light device and the second light device, the third light device having a third rigid housing, a third printed circuit board (PCB) located within the third rigid housing, and a third plurality of light sources electrically coupled to the PCB, wherein the third plurality of light sources are at least partially encompassed within the third rigid housing and thereby protrude through the third rigid housing, and wherein a first portion of the third plurality of light sources emits near infrared light and a second portion of the third plurality of light sources emits red light; and a fourth light device arranged and configured to be detachably coupled to at least one of the first light device, the second light device, and the third light device, the fourth light device having a fourth rigid housing, a fourth printed circuit board (PCB) located within the fourth rigid housing, and a fourth plurality of light sources electrically coupled to the PCB, wherein the fourth plurality of light sources are at least partially encompassed within the fourth rigid housing and thereby protrude through the fourth rigid housing, and wherein a first portion of the fourth plurality of light sources emits near infrared light and a second portion of the fourth plurality of light sources emits red light.

A photobiomodulation therapy system, comprising: a first light device comprising a first rigid housing, a first printed circuit board (PCB) located within the first rigid housing, and a first plurality of light sources electrically coupled to the first PCB, wherein the first plurality of light sources are at least partially encompassed within the first rigid housing and thereby protrude through the first rigid housing, and wherein a first portion of the first plurality of light sources emits near infrared light and a second portion of the first plurality of light sources emits red light; a second light device arranged and configured to be detachably coupled to the first light device, the second light device having a second rigid housing, a second printed circuit board (PCB) located within the second rigid housing, and a second plurality of light sources electrically coupled to the second PCB, wherein the second plurality of light sources are at least partially encompassed within the second rigid housing and thereby protrude through the second rigid housing, and wherein a first portion of the second plurality of light sources emits near infrared light and a second portion of the second plurality of light sources emits red light; a third light device arranged and configured to be detachably coupled to at least one of the first light device and the second light device, the third light device having a third rigid housing, a third printed circuit board (PCB) located within the third rigid housing, and a third plurality of light sources electrically coupled to the PCB, wherein the third plurality of light sources are at least partially encompassed within the third rigid housing and thereby protrude through the third rigid housing, and wherein a first portion of the third plurality of light sources emits near infrared light and a second portion of the third plurality of light sources emits red light; a fourth light device arranged and configured to be detachably coupled to at least one of the first light device, the second light device, and the third light device, the fourth light device having a fourth rigid housing, a fourth printed circuit board (PCB) located within the fourth rigid housing, and a fourth plurality of light sources electrically coupled to the PCB, wherein the fourth plurality of light sources are at least partially encompassed within the fourth rigid housing and thereby protrude through the fourth rigid housing, and wherein a first portion of the fourth plurality of light sources emits near infrared light and a second portion of the fourth plurality of light sources emits red light; and a mobile stand having at least one vertical arm arranged and configured to couple to a large bracket, the mobile stand comprising at least one horizontal leg arranged and configured to contact a floor surface.

In some embodiments, the at least one vertical arm is arranged and configured to rotate about a first axis and a second axis, wherein the second axis is different than the first axis. In some embodiments, the at least one vertical arm is arranged and configured to position at least one of the first light device, the second light device, the third light device, and the fourth light device.

In some embodiments, the system further comprises a quad bracket arranged and configured to be detachably coupled to the back side of the first rigid housing, the back side of the second rigid housing, the back side of the third rigid housing, and the back side of the fourth rigid housing to thereby detachably couple the first rigid housing, second rigid housing, third rigid housing and fourth rigid housing together, wherein the quad bracket is arranged and configured to be detachably coupled to the mobile stand.

The disclosure includes a light therapy device mounting system comprising an elongate pole including a plurality of holes, the elongate pole comprising a first end and a second end, a mounting device slideably coupled to the elongate pole via at least one hole of the plurality of holes, a top bracket slideably coupled to the first end of the elongate pole, wherein the top bracket comprises a top hook for receiving a top portion of a door, and a bottom bracket slideably coupled to the second end of the elongate pole, wherein the bottom bracket comprises a bottom hook for receiving a bottom portion of the door. In some embodiments, the light therapy device mounting system further comprises a light therapy device detachably coupled to the mounting device, wherein the light therapy device comprises a light therapy device bracket coupled to a back side of the light therapy device. The light therapy device bracket may include a plurality of keyhole brackets configured to receive at least one projection coupled to the mounting device.

In some embodiments, the mounting device comprises a hollow cylinder configured to slideably receive the elongate pole, a mounting device bracket coupled to the hollow cylinder, wherein the mounting device bracket is configured to detachably couple to a light therapy device via a plurality of keyhole brackets of a light therapy device bracket located on a back side of the light therapy device, a clip coupled to the hollow cylinder and the mounting device bracket, wherein the clip is located on a top portion of the mounting device bracket and is configured to retain the mounting device bracket, and a knob coupled to a side portion of the hollow cylinder, wherein the knob comprises a protrusion that extends into an inner portion of the hollow cylinder, and wherein the protrusion is configured to couple to at least one hole of the plurality of holes located on the elongate hole.

The elongate pole may be elongate in a first direction, and the knob may be configured to slide in a second direction perpendicular to the first direction, wherein sliding the knob in the second direction is configured to at least one of release and couple the protrusion to at least one hole of the plurality of holes. In some embodiments, the knob is configured to rotate in a third direction perpendicular to the first direction and the second direction, wherein rotating the knob is configured to at least one of tighten and loosen the knob.

The top bracket may comprise a top cap coupled on the first end of the elongate pole and an extension bar configured to couple the top cap to the top hook. The extension bar may comprise at least one height adjustment hole, and the extension bar may be configured to at least one of raise and lower a height of the top hook. In some embodiments, the bottom bracket comprises a bottom cap coupled on the second end of the elongate pole and an adjustable strap configured to couple the bottom cap to the bottom hook, wherein the adjustable strap is configured to at least one of tighten and loosen.

The disclosure includes a light therapy device mounting system comprising an elongate pole including a plurality of holes, the elongate pole comprising a first end and a second end, a first mounting device slideably coupled to the elongate pole via at least one hole of the plurality of holes, and a bottom base configured to receive the second end of the elongate pole. In some embodiments, the system further comprises a mobile stand comprising a plurality of wheels located on a bottom surface of the mobile stand, wherein the bottom base is configured to couple to a top surface of the mobile stand via a plurality of holes located on the top surface configured to receive at least one coupling mechanism, wherein the top surface is located opposite the bottom surface.

The light therapy device mounting system may further comprise a fixed stand configured to contact a ground surface and comprising a plurality of holes configured to receive at least one coupling mechanism, wherein the bottom base is configured to couple to a top surface of the fixed stand via the at least one coupling mechanism, and a wall bracket snapably coupled to the elongate pole, wherein the wall bracket comprises a plurality of holes configured to receive at least one coupling mechanism to thereby couple the wall bracket to a wall surface. In some embodiments, the fixed stand comprises a plurality of feet located on a bottom surface of the fixed stand, the plurality of feet configured to contact the ground surface, wherein the bottom surface is located opposite the top surface of the fixed stand. The wall bracket may comprise a C-shaped hook located on a first surface of the wall bracket, the C-shaped hook configured to snapably couple to the elongate pole. In some embodiments, the plurality of holes are located on a second surface of the wall bracket opposite the first surface, such that when the wall bracket is snapably coupled to the elongate pole and the wall bracket is coupled to the wall surface via the at least one coupling mechanism, the elongate pole is thereby coupled to the wall surface via the wall bracket.

The mobile stand may be configured to couple to a first bottom base via the plurality of holes located on the top surface of the mobile stand configured to receive the at least one coupling mechanism, wherein the first bottom base is configured to receive a first elongate pole, thereby coupling the first elongate pole to the mobile stand. In some embodiments, the mobile stand is configured to couple to a first bottom base, a second bottom base, and a third bottom base via the plurality of holes located on the top surface of the mobile stand configured to receive the at least one coupling mechanism. The first bottom base may be configured to receive a first elongate pole, the second bottom base may be configured to receive a second elongate pole, and the third bottom base may be configured to receive a third elongate pole, thereby coupling the first elongate pole, the second elongate pole, and the third elongate pole to the mobile stand. The light therapy device mounting system may further comprise at least one brace configured to couple at least one of the first elongate pole, the second elongate pole, and the third elongate pole to at least one adjacent pole.

In some embodiments, the elongate pole comprises a first elongate pole, and the system further comprises a second mounting device slideably coupled to one of the first elongate pole and a second elongate pole, wherein the system is sized and configured to accommodate a plurality of light therapy devices.

The disclosure includes a light therapy device floor stand comprising a floor plate extending along a first direction and a second direction, wherein the second direction is perpendicular to the first direction, a first curved side arm coupled to a top surface of the floor plate wherein the first curved side arm extends along a third direction that is perpendicular to both the first direction and the second direction, wherein the first curved side arm is coupled to a first side of the floor plate, and a second curved side arm coupled to the top surface of the floor plate and coupled to a second side located opposite the first side, the second curved side arm facing the first curved side arm such that the first curved side arm and the second curved side arm open toward one another. In some embodiments, the first curved side arm and the second curved side arm are configured to slideably receive a bottom portion of a light therapy device.

In some embodiments, the top surface of the floor plate comprises an indented portion arranged and configured to receive at least one of a portion of the first curved side arm, a portion of the second curved side arm, and the bottom portion of the light therapy device. The first curved side arm and the second curved side arm may be configured to couple to the floor plate in the indented portion.

The disclosure also includes a light therapy device mounting kit comprising a first spacer including a first flat base of the first spacer, a first leg of the first spacer, and a second leg of the first spacer. In some embodiments, the first leg of the first spacer protrudes away from the first flat base along a first direction, and is arranged and configured to slideably engage a first cavity located within a first light therapy device. The second leg of the first spacer may protrude away from the first flat base along the first direction, and may be spaced from the first leg of the first spacer. In some embodiments, the second leg of the first spacer is arranged and configured to slideably engage a first cavity located within a second light therapy device.

In many embodiments, the first flat base is elongate along a vertical direction perpendicular to the first direction, and the first spacer defines a first vertical spacer. The first leg of the first vertical spacer may be spaced from the second leg of the first vertical spacer along the vertical direction.

In some embodiments, the light therapy device mounting kit further comprises a first square spacer including a first flat base of the first square spacer elongate along the vertical direction and a horizontal direction perpendicular to the first direction and the vertical direction. The first square spacer may also include a first leg of the first square spacer protruding away from the first flat base of the first square spacer along the first direction, the first leg of the first square spacer arranged and configured to slideably engage a second cavity located within the first light therapy device, a second leg of the first square spacer protruding away from the first flat base of the first square spacer along the first direction, the second leg of the first square spacer spaced from the first leg of the first square spacer, the second leg of the first square spacer arranged and configured to slideably engage a second cavity located within the second light therapy device, a third leg of the first square spacer protruding away from the first flat base of the first square spacer along the first direction, the third leg of the first square spacer spaced from the first leg of the first square spacer and the second leg of the first square spacer, the third leg of the first square spacer arranged and configured to slideably engage a first cavity located within a third light therapy device, and a fourth leg protruding away from the first flat base of the first square spacer along the first direction, the fourth leg of the first square spacer spaced from the first leg of the first square spacer, the second leg of the first square spacer, and the third leg of the first square spacer, the fourth leg of the first square spacer arranged and configured to slideably engage a first cavity located within a fourth light therapy device.

The first leg of the first square spacer may be spaced from the second leg of the first square spacer along the vertical direction, and may be spaced from the third leg of the first square spacer along the horizontal direction. In some embodiments, the second leg of the first square spacer is spaced from the first leg of the first square spacer along the vertical direction, and spaced from the fourth leg of the first square spacer along the horizontal direction. The third leg of the first square spacer may be spaced from the first leg of the first square spacer along the horizontal direction, and may be spaced from the fourth leg of the first square spacer along the vertical direction. In some embodiments, the fourth leg of the first square spacer is spaced from the second leg of the first square spacer along the horizontal direction, and spaced from the third leg of the first square spacer along the vertical direction.

In some embodiments, the light therapy device mounting kit further comprises a first horizontal spacer including a first flat base of the first horizontal spacer elongate along the horizontal direction, a first leg of the first horizontal spacer protruding away from the first flat base of the first horizontal spacer along the first direction, the first leg of the first horizontal spacer arranged and configured to slideably engage a third cavity located within the second light therapy device, and a second leg of the first horizontal spacer protruding away from the first flat base of the first horizontal spacer along the first direction, the second leg of the first horizontal spacer spaced from the first leg of the first horizontal spacer, the second leg of the first horizontal spacer arranged and configured to slideably engage a second cavity within the fourth light therapy device. The first leg of the first horizontal spacer may be spaced from the second leg of the first horizontal spacer along the horizontal direction.

In some embodiments, the light therapy device mounting kit further comprises a first O-ring arranged and configured to slideably couple to one of the first leg of the first vertical spacer and the second leg of the first vertical spacer. The light therapy device mounting kit may also comprise a second vertical spacer including a first flat base of the second vertical spacer elongate along the vertical direction, a first leg of the second vertical spacer protruding away from the first flat base along the first direction, the first leg of the second vertical spacer arranged and configured to slideably engage a first cavity located within a third light therapy device, and a second leg of the second vertical spacer protruding away from the first flat base along the first direction, the second leg of the second vertical spacer spaced from the first leg of the second vertical spacer, the second leg of the second vertical spacer arranged and configured to slideably engage a first cavity located within a fourth light therapy device. In some embodiments, the light therapy device mounting kit further comprises a second O-ring and a third O-ring, wherein each of the second O-ring and the third O-ring is arranged and configured to slideably couple to at least one leg of the first vertical spacer and at least one leg of the second vertical spacer.

A first square spacer may include a first flat base of the first square spacer elongate along the vertical direction and a horizontal direction perpendicular to the first direction and the vertical direction, a first leg of the first square spacer protruding away from the first flat base of the first square spacer along the first direction, the first leg of the first square spacer arranged and configured to slideably engage a second cavity located within the first light therapy device, a second leg of the first square spacer protruding away from the first flat base of the first square spacer along the first direction, the second leg of the first square spacer spaced from the first leg of the first square spacer, the second leg of the first square spacer arranged and configured to slideably engage a second cavity located within the second light therapy device, a third leg of the first square spacer protruding away from the first flat base of the first square spacer along the first direction, the third leg of the first square spacer spaced from the first leg of the first square spacer and the second leg of the first square spacer, the third leg of the first square spacer arranged and configured to slideably engage a second cavity located within the third light therapy device, and a fourth leg protruding away from the first flat base of the first square spacer along the first direction, the fourth leg of the first square spacer spaced from the first leg of the first square spacer, the second leg of the first square spacer, and the third leg of the first square spacer, the fourth leg of the first square spacer arranged and configured to slideably engage a second cavity located within the fourth light therapy device.

In some embodiments, the mounting kit further comprises a first horizontal spacer including a first flat base of the first horizontal spacer elongate along the horizontal direction, a first leg of the first horizontal spacer protruding away from the first flat base of the first horizontal spacer along the first direction, the first leg of the first horizontal spacer arranged and configured to slideably engage a third cavity located within the first light therapy device, and a second leg of the first horizontal spacer protruding away from the first flat base of the first horizontal spacer along the first direction, the second leg of the first horizontal spacer spaced from the first leg of the first horizontal spacer, the second leg of the first horizontal spacer arranged and configured to slideably engage a third cavity within the third light therapy device. The mounting kit may further comprise a second horizontal spacer including a first flat base of the second horizontal spacer elongate along the horizontal direction, a first leg of the second horizontal spacer protruding away from the first flat base of the second horizontal spacer along the first direction, the first leg of the second horizontal spacer arranged and configured to slideably engage a third cavity located within the second light therapy device, and a second leg of the second horizontal spacer protruding away from the first flat base of the second horizontal spacer along the first direction, the second leg of the second horizontal spacer spaced from the first leg of the second horizontal spacer, the second leg of the second horizontal spacer arranged and configured to slideably engage a third cavity within the fourth light therapy device.

In some embodiments, the mounting kit includes a fourth O-ring, a fifth O-ring, a sixth O-ring, and a seventh O-ring, wherein each of the fourth O-ring, the fifth O-ring, the sixth O-ring, and the seventh O-ring is arranged and configured to slideably couple to at least one leg of the first square spacer, at least one leg of the first horizontal spacer, and at least one leg of the second horizontal spacer. The mounting kit may include a second vertical spacer including a first flat base of the second vertical spacer elongate along the vertical direction, a first leg of the second vertical spacer protruding away from the first flat base along the first direction, the first leg of the second vertical spacer arranged and configured to slideably engage a first cavity located within a fifth light therapy device, and a second leg of the second vertical spacer protruding away from the first flat base along the first direction, the second leg of the second vertical spacer spaced from the first leg of the second vertical spacer, the second leg of the second vertical spacer arranged and configured to slideably engage a first cavity located within a sixth light therapy device.

In some embodiments, the mounting kit comprises a second square spacer including a first flat base of the first square spacer elongate along the vertical direction and a horizontal direction perpendicular to the first direction and the vertical direction, a first leg of the first square spacer protruding away from the first flat base of the first square spacer along the first direction, the first leg of the first square spacer arranged and configured to slideably engage a second cavity located within the third light therapy device, a second leg of the first square spacer protruding away from the first flat base of the first square spacer along the first direction, the second leg of the first square spacer spaced from the first leg of the first square spacer, the second leg of the first square spacer arranged and configured to slideably engage a third cavity located within the fourth light therapy device, a third leg of the first square spacer protruding away from the first flat base of the first square spacer along the first direction, the third leg of the first square spacer spaced from the first leg of the first square spacer and the second leg of the first square spacer, the third leg of the first square spacer arranged and configured to slideably engage a second cavity located within the fifth light therapy device, and a fourth leg protruding away from the first flat base of the first square spacer along the first direction, the fourth leg of the first square spacer spaced from the first leg of the first square spacer, the second leg of the first square spacer, and the third leg of the first square spacer, the fourth leg of the first square spacer arranged and configured to slideably engage a second cavity located within the sixth light therapy device.

The mounting kit may comprise a second horizontal spacer including a first flat base of the second horizontal spacer elongate along the horizontal direction, a first leg of the second horizontal spacer protruding away from the first flat base of the second horizontal spacer along the first direction, the first leg of the second horizontal spacer arranged and configured to slideably engage a third cavity located within the first light therapy device, and a second leg of the first horizontal spacer protruding away from the first flat base of the first horizontal spacer along the first direction, the second leg of the first horizontal spacer spaced from the first leg of the first horizontal spacer, the second leg of the first horizontal spacer arranged and configured to slideably engage a third cavity within the third light therapy device. In some embodiments, the mounting kit further comprises a third horizontal spacer including a first flat base of the third horizontal spacer elongate along the horizontal direction, a first leg of the third horizontal spacer protruding away from the first flat base of the third horizontal spacer along the first direction, the first leg of the third horizontal spacer arranged and configured to slideably engage a third cavity located within the fourth light therapy device, and a second leg of the third horizontal spacer protruding away from the first flat base of the third horizontal spacer along the first direction, the second leg of the third horizontal spacer spaced from the first leg of the third horizontal spacer, the second leg of the third horizontal spacer arranged and configured to slideably engage a third cavity within the sixth light therapy device. The mounting kit may also comprise a fourth horizontal spacer including a first flat base of the fourth horizontal spacer elongate along the horizontal direction, a first leg of the fourth horizontal spacer protruding away from the first flat base of the fourth horizontal spacer along the first direction, the first leg of the fourth horizontal spacer arranged and configured to slideably engage a fourth cavity located within the third light therapy device, and a second leg of the fourth horizontal spacer protruding away from the first flat base of the fourth horizontal spacer along the first direction, the second leg of the fourth horizontal spacer spaced from the first leg of the fourth horizontal spacer, the second leg of the fourth horizontal spacer arranged and configured to slideably engage a third cavity within the fifth light therapy device.

The disclosure includes a light therapy system comprising a first light therapy device comprising a first front treatment surface and a first back surface located opposite the first front treatment surface and a second light therapy device comprising a second front treatment surface and a second back surface located opposite the second front treatment surface. The system may further comprise a first vertical spacer including a first flat base of the first vertical spacer elongate along a vertical direction, a first leg of the first vertical spacer protruding away from the first flat base along a first direction that is perpendicular to the vertical direction, the first leg of the first vertical spacer arranged and configured to slideably engage a first cavity located within the first light therapy device, and a second leg of the first vertical spacer protruding away from the first flat base along the first direction, the second leg of the first vertical spacer spaced from the first leg of the first vertical spacer, the second leg of the first vertical spacer arranged and configured to slideably engage a first cavity located within a second light therapy device. In some embodiments, the system comprises a second vertical spacer including a first flat base of the second vertical spacer elongate along the vertical direction, a first leg of the second vertical spacer protruding away from the first flat base along the first direction, the first leg of the second vertical spacer arranged and configured to slideably engage a second cavity located within the first light therapy device, and a second leg of the second vertical spacer protruding away from the first flat base along the first direction, the second leg of the second vertical spacer spaced from the first leg of the second vertical spacer, the second leg of the second vertical spacer arranged and configured to slideably engage a second cavity located within the second light therapy device.

In some embodiments, the first light therapy device defines a first size and the second light therapy device defines a second size. The first size may be smaller than the second size, and the system may further comprise a first O-ring arranged and configured to slideably couple to the first leg of the first vertical spacer, such that the first O-ring may be arranged and configured to couple within the first cavity of the first light therapy device, and a second O-ring arranged and configured to slideably couple to the first leg of the second vertical spacer, such that the second O-ring may be arranged and configured to couple within the second cavity of the first light therapy device.

In many embodiments, the light therapy system comprises a third light therapy device comprising a third front treatment surface and a third back surface located opposite the third front treatment surface, wherein the first leg of the second vertical spacer is arranged and configured to slideably engage a first cavity located within the third light therapy device, and a fourth light therapy device comprising a fourth front treatment surface and a fourth back surface located opposite the fourth front treatment surface, wherein the second leg of the second vertical spacer is arranged and configured to slideably engage a first cavity located within the fourth light therapy device.

The system may also comprise a first square spacer including a first flat base of the first square spacer elongate along the vertical direction and a horizontal direction perpendicular to the first direction and the vertical direction, a first leg of the first square spacer protruding away from the first flat base of the first square spacer along the first direction, the first leg of the first square spacer arranged and configured to slideably engage a second cavity located within the first light therapy device, a second leg of the first square spacer protruding away from the first flat base of the first square spacer along the first direction, the second leg of the first square spacer spaced from the first leg of the first square spacer, the second leg of the first square spacer arranged and configured to slideably engage a second cavity located within the second light therapy device, a third leg of the first square spacer protruding away from the first flat base of the first square spacer along the first direction, the third leg of the first square spacer spaced from the first leg of the first square spacer and the second leg of the first square spacer, the third leg of the first square spacer arranged and configured to slideably engage a second cavity located within the third light therapy device, and a fourth leg protruding away from the first flat base of the first square spacer along the first direction, the fourth leg of the first square spacer spaced from the first leg of the first square spacer, the second leg of the first square spacer, and the third leg of the first square spacer, the fourth leg of the first square spacer arranged and configured to slideably engage a second cavity located within the fourth light therapy device.

In some embodiments, the system comprises a first horizontal spacer including a first flat base of the first horizontal spacer elongate along the horizontal direction, a first leg of the first horizontal spacer protruding away from the first flat base of the first horizontal spacer along the first direction, the first leg of the first horizontal spacer arranged and configured to slideably engage a third cavity located within the first light therapy device, and a second leg of the first horizontal spacer protruding away from the first flat base of the first horizontal spacer along the first direction, the second leg of the first horizontal spacer spaced from the first leg of the first horizontal spacer, the second leg of the first horizontal spacer arranged and configured to slideably engage a third cavity within the third light therapy device. The system may also comprise a second horizontal spacer including a first flat base of the second horizontal spacer elongate along the horizontal direction, a first leg of the second horizontal spacer protruding away from the first flat base of the second horizontal spacer along the first direction, the first leg of the second horizontal spacer arranged and configured to slideably engage a third cavity located within the second light therapy device, and a second leg of the second horizontal spacer protruding away from the first flat base of the second horizontal spacer along the first direction, the second leg of the second horizontal spacer spaced from the first leg of the second horizontal spacer, the second leg of the second horizontal spacer arranged and configured to slideably engage a third cavity within the fourth light therapy device.

In many embodiments, the first light therapy device defines a first size, the second light therapy device defines a second size, the third light therapy device defines a third size, and the fourth light therapy device defines a fourth size, wherein the first size and third size are smaller than the second size and fourth size. The system may further comprise a first O-ring arranged and configured to slideably couple to the first leg of the first vertical spacer, such that the first O-ring is arranged and configured to couple within the first cavity of the first light therapy device, a second O-ring arranged and configured to slideably couple to the first leg of the second vertical spacer, such that the second O-ring is arranged and configured to couple within the first cavity of the third light therapy device, a third O-ring arranged and configured to slideably couple to the first leg of the first square spacer, such that the third O-ring is arranged and configured to couple within the second cavity of the first light therapy device, a fourth O-ring arranged and configured to slideably couple to the third leg of the first square spacer, such that the fourth O-ring is arranged and configured to couple within the second cavity of the third light therapy device, a fifth O-ring arranged and configured to slideably couple to the first leg of the first horizontal spacer, such that the fifth O-ring is arranged and configured to couple within the third cavity of the first light therapy device, and a sixth O-ring arranged and configured to slideably couple to the second leg of the first horizontal spacer, such that the sixth O-ring is arranged and configured to couple within the third cavity of the third light therapy device.

In some embodiments, the system comprises a fifth light therapy device comprising a fifth front treatment surface and a fifth back surface located opposite the fifth front treatment surface, wherein the first leg of the second vertical spacer is arranged and configured to slideably engage a first cavity located within the fifth light therapy device, and a sixth light therapy device comprising a sixth front treatment surface and a sixth back surface located opposite the sixth front treatment surface, wherein the second leg of the second vertical spacer is arranged and configured to slideably engage a first cavity located within the sixth light therapy device. The system may further comprise the first square spacer, wherein the first leg of the first square spacer is arranged and configured to slideably engage a second cavity located within the first light therapy device, the second leg of the first square spacer is arranged and configured to slideably engage a second cavity located within the second light therapy device, the third leg of the first square spacer is arranged and configured to slideably engage a first cavity located within the third light therapy device, and the fourth leg of the first square spacer is arranged and configured to slideably engage a first cavity located within the fourth light therapy device.

The system may also comprise a second square spacer including a first flat base of the second square spacer elongate along the vertical direction and a horizontal direction perpendicular to the first direction and the vertical direction, a first leg of the second square spacer protruding away from the first flat base of the second square spacer along the first direction, the first leg of the second square spacer arranged and configured to slideably engage a second cavity located within the third light therapy device, a second leg of the second square spacer protruding away from the first flat base of the second square spacer along the first direction, the second leg of the second square spacer spaced from the first leg of the second square spacer, the second leg of the second square spacer arranged and configured to slideably engage a second cavity located within the fourth light therapy device, a third leg of the second square spacer protruding away from the first flat base of the second square spacer along the first direction, the third leg of the second square spacer spaced from the first leg of the second square spacer and the second leg of the second square spacer, the third leg of the second square spacer arranged and configured to slideably engage a second cavity located within the fifth light therapy device, and a fourth leg protruding away from the first flat base of the second square spacer along the first direction, the fourth leg of the second square spacer spaced from the first leg of the second square spacer, the second leg of the second square spacer, and the third leg of the second square spacer, the fourth leg of the second square spacer arranged and configured to slideably engage a second cavity located within the sixth light therapy device.

In some embodiments, the system comprises a third horizontal spacer including a first flat base of the third horizontal spacer elongate along the horizontal direction, a first leg of the third horizontal spacer protruding away from the first flat base of the third horizontal spacer along the first direction, the first leg of the third horizontal spacer arranged and configured to slideably engage a fourth cavity located within the third light therapy device, and a second leg of the third horizontal spacer protruding away from the first flat base of the third horizontal spacer along the first direction, the second leg of the third horizontal spacer spaced from the first leg of the third horizontal spacer, the second leg of the third horizontal spacer arranged and configured to slideably engage a third cavity within the fifth light therapy device. The system may further comprise a fourth horizontal spacer including a first flat base of the fourth horizontal spacer elongate along the horizontal direction, a first leg of the fourth horizontal spacer protruding away from the first flat base of the fourth horizontal spacer along the first direction, the first leg of the fourth horizontal spacer arranged and configured to slideably engage a fourth cavity located within the fourth light therapy device, and a second leg of the fourth horizontal spacer protruding away from the first flat base of the fourth horizontal spacer along the first direction, the second leg of the fourth horizontal spacer spaced from the first leg of the fourth horizontal spacer, the second leg of the fourth horizontal spacer arranged and configured to slideably engage a third cavity within the sixth light therapy device.

### BRIEF DESCRIPTION OF THE DRAWINGS

These and other features, aspects, and advantages are described below with reference to the drawings, which are intended to illustrate, but not to limit, the invention. In the drawings, like reference characters denote corresponding features consistently throughout similar embodiments.

FIG. 1 illustrates a perspective view of an embodiment of a light therapy system, according to some embodiments.

FIG. 2 illustrates a perspective view of a light therapy system, specifically how a first housing mechanically couples with a second housing, according to some embodiments.

FIG. 3 illustrates a back view of a light therapy system, specifically how a first light device electrically couples with a second light device, according to some embodiments.

FIG. 4 illustrates a perspective view of a light therapy system having four light devices mechanically coupled, according to some embodiments.

FIG. 5 illustrates a back view of a light therapy system in which a first light device is electrically coupled to a first power outlet and a second light device, and a third light device electrically coupled to a second power outlet and a fourth light device, according to some embodiments.

FIG. 6 illustrates a back view of a light therapy system in which a first light device is electrically coupled to a first power outlet and a second light device, the second light device is also electrically coupled to a third light device, and the third light device is also electrically coupled to a fourth light device, according to some embodiments.

FIG. 7 illustrates a perspective view of a light therapy system having six light devices mechanically coupled together, according to some embodiments.

FIG. 8 illustrates a back view of a light therapy system, specifically showing six light devices mechanically and electrically coupled together, according to some embodiments.

FIG. 9 illustrates a back view of another light therapy system, specifically showing six light devices mechanically and electrically coupled together, according to some embodiments.

FIG. 10 illustrates a perspective view of a light therapy system in which a first light device is mechanically coupled with a second light device in a top-to-bottom orientation, according to some embodiments.

FIG. 11 illustrates a back view of a light therapy system in which a first light device is mechanically coupled to a second light device in a side-by-side orientation, according to some embodiments.

FIG. 12 illustrates a front view of a light therapy system that has four light devices mechanically coupled and is mounted to a door via a first and a second door stand, according to some embodiments.

FIG. 13 illustrates a back view of a light therapy system in which a first light device is mechanically coupled to a second light device via a first and a second bracket, and a third light device is mechanically coupled to a fourth light device via a third and a fourth bracket, according to some embodiments.

FIG. 14 illustrates a perspective view of a light therapy system mounted to a door, specifically showing a third and fourth housing mechanically coupled with a first and second housing, according to some embodiments.

FIG. 15 illustrates a back view of a light therapy system in which first, second, third, and fourth light devices are mechanically coupled via a first bracket, a second bracket, and a quad bracket, according to some embodiments.

FIG. 16 illustrates a back view of a light therapy system in which first, second, third, and fourth light devices are mechanically coupled via a first bracket, a second bracket, and a quad bracket, whereby the quad bracket is mechanically coupled with a mobile stand, according to some embodiments.

FIG. 17 illustrates a perspective view of a mobile stand, according to some embodiments.

FIG. 18 illustrates a perspective view of one possible embodiment of a light therapy system mounted to a mobile stand that is capable of rotation on two axes, according to some embodiments.

FIG. 19 illustrates a perspective view of a light therapy system mounted to a mobile stand in which a first and second light device have a first form factor, and a third and fourth light device have a second form factor, according to some embodiments.

FIG. 20 illustrates a back view of a light therapy system in which first, second, third, fourth, fifth, and sixth light devices are mechanically coupled via a first bracket, a second bracket, and a large bracket, according to some embodiments.

FIG. 21 illustrates a back view of a light therapy system in which first, second, third, fourth, fifth, and sixth light devices are mechanically coupled via a first bracket, a second bracket, and a large bracket that is mechanically coupled with a mobile stand, according to some embodiments.

FIG. 22 illustrates a perspective view of a light therapy system that includes a mini stand, according to some embodiments.

FIG. 23 illustrates a perspective view of a light therapy system that includes a wall mount for attaching the system to a substrate, such as a wall, according to some embodiments.

FIG. 24 illustrates a perspective view of a partial light device with the lights protruding from the rigid housing, according to some embodiments.

FIG. 25 illustrates a side cross-sectional view of a light device whereby the lights are at least partially encompassed within the rigid housing and at least partially protruding from the rigid housing, according to some embodiments.

FIG. 26 illustrates a front view of a light therapy device mounting system coupled to a door, according to some embodiments.

FIG. 27 illustrates a perspective view of a light therapy device mounting system coupled to a door, according to some embodiments.

FIGS. 28A and 28B illustrate front and back views, respectively, of a top bracket, according to some embodiments.

FIGS. 29A and 29B illustrate front and back views, respectively, of a bottom bracket, according to some embodiments.

FIG. 30 illustrates a perspective view of a mounting device, according to some embodiments.

FIG. 31 illustrates a top view of a mounting device, according to some embodiments.

FIG. 32 illustrates a perspective view of a light therapy device mounting system, according to some embodiments.

FIG. 33 illustrates a perspective view of a light therapy device bracket, according to some embodiments.

FIG. 34 illustrates a perspective view of a light therapy device mounting system, according to some embodiments.

FIG. 35 illustrates a perspective view of a mobile stand, according to some embodiments.

FIG. 36 illustrates a front view of a plurality of light therapy devices coupled to a mobile stand, according to some embodiments.

FIG. 37 illustrates a side view of a plurality of light therapy devices coupled to a mobile stand, according to some embodiments.

FIG. 38A illustrates a perspective view of a mobile stand, according to some embodiments.

FIGS. 38B and 38C illustrate perspective views of a mobile stand coupled to a plurality of elongate poles, according to some embodiments.

FIG. 39 illustrates a front view of a plurality of light therapy devices coupled to a mobile stand, according to some embodiments.

FIG. 40 illustrates a side view of a plurality of light therapy devices coupled to a mobile stand, according to some embodiments.

FIGS. 41A, 41B, 41C, and 41D illustrate various embodiments of at least one brace.

FIG. 42 illustrates a back view of a plurality of light therapy devices coupled to a mobile stand, according to some embodiments.

FIG. 43 illustrates a perspective view of a fixed stand, according to some embodiments.

FIG. 44A, 44B, and 44C illustrate perspective views of a light therapy device mounting system, including a wall bracket, according to some embodiments.

FIG. 45 illustrates a front view of a plurality of light therapy devices coupled to a fixed stand, according to some embodiments.

FIG. 46 illustrates a side view of a plurality of light therapy devices coupled to a fixed stand, according to some embodiments.

FIG. 47 illustrates a perspective view of a light therapy device floor stand, according to some embodiments.

FIG. 48 illustrates a perspective view of a light therapy device coupled to a light therapy device floor stand, according to some embodiments.

FIGS. 49A, 49B, and 49C illustrates perspective views of a light therapy device table stand, according to some embodiments.

FIG. 50 illustrates a perspective view of a light therapy device coupled to a light therapy device table stand, according to some embodiments.

FIGS. 51A and 51B illustrate top and bottom perspective views, respectively, of a vertical spacer, according to some embodiments.

FIGS. 52A and 52B illustrate top and bottom perspective views, respectively, of a square spacer, according to some embodiments.

FIGS. 53A and 53B illustrate top and bottom perspective views, respectively, of a horizontal spacer, according to some embodiments.

FIGS. 54 and 55 illustrates back perspective views of a light therapy device mounting kit, according to some embodiments.

FIGS. 56, 57, 58, 59, 60, 61, 62, 63, and 64 illustrate back views of a light therapy device mounting kit, according to some embodiments.

### DETAILED DESCRIPTION

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any system or method disclosed herein, the acts or operations of the system or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

### Introduction

Photobiomodulation therapy provides an alternative option for treating many common ailments and diseases. For example, when the human body is exposed to red light, blue light, green light, and/or near infrared light, subjects can expect to see improvement in multiple skin conditions, weight loss, muscle recovery, sexual performance, joint pain, and thyroid function. Instead of using prescription medications to solve these many problems, light therapy can be used in place of these traditional remedies to achieve safe and effective results.

Light therapy has been adopted by many top professionals, but oftentimes the light therapy devices and systems used are not big enough to treat an entire body at once. Accordingly, many embodiments described herein enable two or more light therapy devices to be coupled together in various ways. In doing so, the area of treatment can be expanded to reduce the time and number of treatments to achieve the desired results.

An objective of the present invention is to provide a variety of accessories to support (e.g., hang, mount, hold in an upright position, etc.) at least one light therapy device. Of the embodiments described herein are different components, some of which may be used interchangeably with other components. For example, the elongate pole may be used with a mobile stand, a fixed stand, a door mounting system, and/or a wall mounting system. A single light therapy device or multiple light therapy devices may be supported by any of the accessory devices/systems described herein.

### LIST OF REFERENCE NUMERALS

10—light therapy system
12—light device
14—housing
16—plurality of lights
17—lights
17a—portion of lights protruding from housing
17b—portion of lights at least partially encompassed within housing
18—power management system
20—power cord
22—power outlet
30—aperture
32—bottom surface
34—attachment post
36—top surface
38—bracket
39—attachment member
40—back side
42—door stand
44—quad bracket
50—mobile stand
52—vertical arm
54—horizontal leg
56—form factor
58—wall mount
59—mini stand
60—first axis
62—second axis
64—large bracket
100—light therapy device mounting system
101—bottom portion (light therapy device)
102—light therapy device
104—light therapy device table stand
106—back side
108—light therapy device
110—base
112—arm
114—back bracket
120—elongate pole
140—plurality of holes
160—first end
180—second end
200—mounting device
220a—top bracket
220b—bottom bracket
240a—top hook
240b—bottom hook
260a—top portion (of door)
260b—bottom portion (of door)
280—door
300—light therapy device
300a—plurality of light therapy devices
320—light therapy device bracket
340—back side (of light therapy device)
360—plurality of keyhole brackets
370—at least one projection
380—hollow cylinder
400—mounting device bracket
420—clip
440—top portion (of mounting device bracket)
460—knob
480—protrusion
500—inner portion (of hollow cylinder)
520a—top cap
520b—bottom cap
540—extension bar
560—adjustable strap
580—bottom base
600—mobile stand
620—plurality of wheels
640a—bottom surface (mobile stand)
640b—bottom surface (fixed stand)
660a—top surface (mobile stand)
660b—top surface (fixed stand)
680a—plurality of holes (mobile stand)
680b—plurality of holes (fixed stand)
700—at least one coupling mechanism
720—fixed stand
740—ground surface
760—wall bracket
780—plurality of holes (wall bracket)
800—wall surface
820—plurality of feet
840—C-shaped hook
860a—first surface (wall bracket)
860b—second surface (wall bracket)
880—at least one brace
900—light therapy device floor stand
920—floor plate
940a—first curved side arm
940b—second curved side arm
960—top surface
980a—first side (floor plate)
980b—second side (floor plate)
1000—light therapy device mounting kit
1002—first spacer
1004—first flat base
1006—first leg
1008—cavity (first light therapy device)
1010—first light therapy device
1012—second leg
1014—cavity (second light therapy device)
1016—second light therapy device
1018—vertical spacer
1020—square spacer
1022—first flat base (square spacer)
1024—first leg
1026—second leg
1028—third leg
1030—fourth leg
1032—cavity (third light therapy device)
1034—third light therapy device
1036—cavity (fourth light therapy device)
1038—fourth light therapy device
1040—horizontal spacer
1042—first flat base (horizontal spacer)
1044—first leg
1046—second leg
1048—O-ring
1050—cavity (fifth light therapy device)
1052—fifth light therapy device
1054—cavity (sixth light therapy device)
1056—sixth light therapy device
1058—first size
1060—second size

### Modular System Embodiments

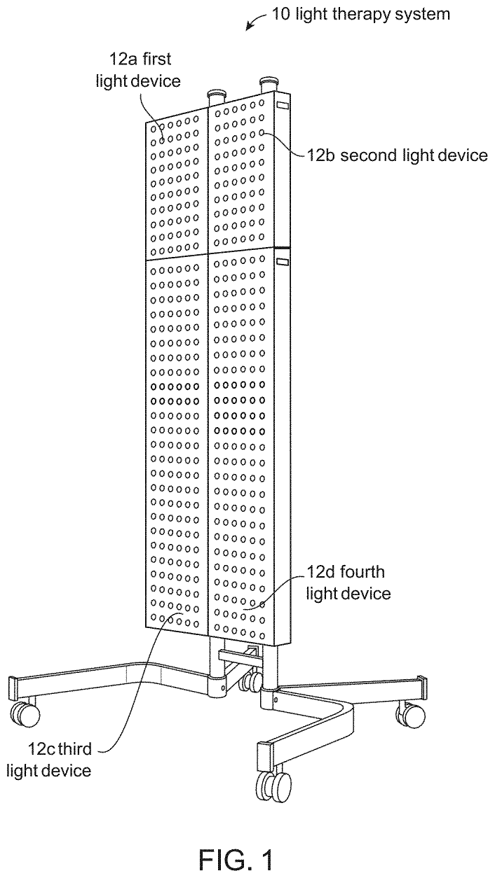

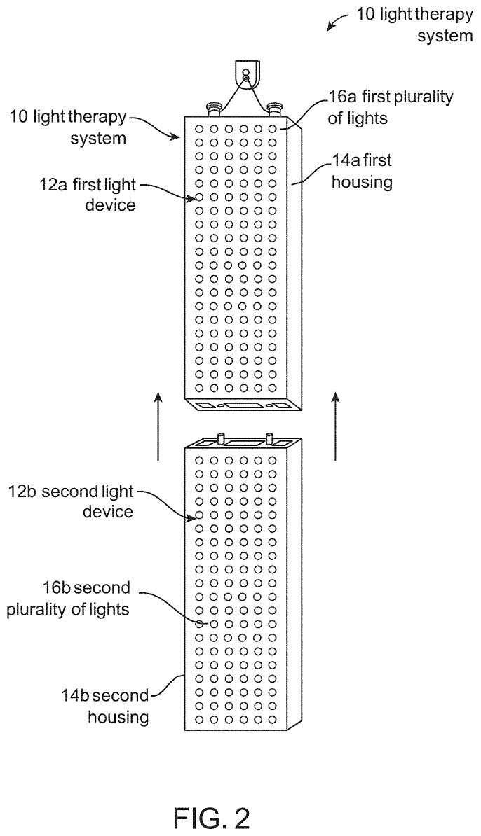

The disclosure includes a light therapy system 10 that includes two or more light devices 12. For example, in some embodiments as shown in FIG. 1, the system 10 includes four light devices, such as a first light device 12a, a second light device 12b, a third light device 12c, and a fourth light device 12d. As will be shown and described, the four light devices 12 may be electrically and mechanically coupled together. The light therapy system 10 may further include an auxiliary mounting device, such as a mobile stand and/or a door stand, to assist in transporting and positioning the system 10. In order to describe specific features and components of the light therapy system 10, we now refer to FIG. 2, which illustrates a perspective view of an embodiment of a system 10 having the light devices arranged in a top-to-bottom orientation. As shown, the system 10 may include a first light device 12a comprising a first housing 14a and a first plurality of lights 16a, and a second light device 12b comprising a second housing 14b and a second plurality of lights 16b. In many embodiments, the first housing and the second housing are constructed of rigid materials. As such, in many embodiments, the housing(s) is(are) rigid. The second light device 12b may be arranged and configured to couple to the first light device 12a. In many embodiments, the first rigid housing 14a is configured to be detachably coupled to the second rigid housing 14b. Both the first plurality of lights 16a and the second plurality of lights 16b are arranged and configured to emit red light, blue light, green light, and/or near infrared light. In some embodiments, the plurality of lights 16a and 16b may comprise a plurality of light emitting diodes (LED). Although LEDs are specifically mentioned herein, any light source capable of emitting red light, blue light, green light, and/or near infrared light may be appropriate. Furthermore, each light of the plurality of lights may be configured to emit both red light, blue light, green light, and/or near infrared light, or each light may be configured to only emit red light, blue light, green light, and/or near infrared light.

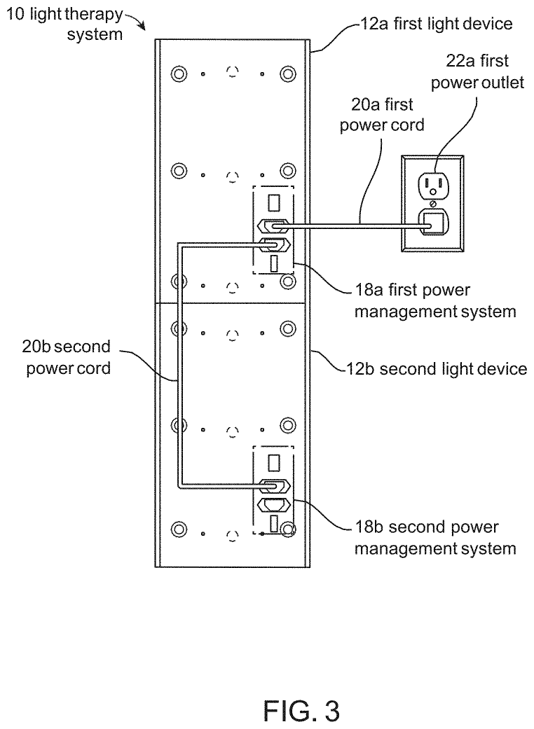

In some embodiments, the first light device 12a is arranged and configured to electrically couple to the second light device 12b. FIG. 3 illustrates one embodiment of how this may be achieved. The first light device 12a may comprise a first power management system 18a, which is electrically coupled to the first plurality of lights 16a. Likewise, the second light device 12b may comprise a second power management system 18b, which is electrically coupled to the second plurality of lights 16b. In some embodiments, the power management systems 18 may include an input port configured to receive a cord to thereby transfer power into the light devices 12, and an output port configured to transfer power out of the light device 12. Moreover, FIG. 3 illustrates the light devices arranged in a top-to-bottom orientation.

FIG. 3 further illustrates an embodiment which has a first power cord 20a electrically coupled to the first power management system 18a and a first power outlet 22a. Furthermore, FIG. 3 illustrates a second power cord 20b electrically coupled to the second power management system 18b. In such embodiments, power may pass through the first light device 12a via the second power cord 20b such that the second light device 12b will have sufficient power to perform as expected.

The power cord(s) 20 may also be configured to send signals from one light device 12 to another. This can be advantageous to ensure that multiple light devices 12 perform the same task. For example, a user may wish to use only red light, blue light, green light, and/or near infrared light, or any combination of lights. In some embodiments, a user may input instructions into the first light device 12a, and the signal will be carried through by the second power cord 20b to the second light device 12b. In this regard, the second light device 12b will be paired to the first light device 12a.

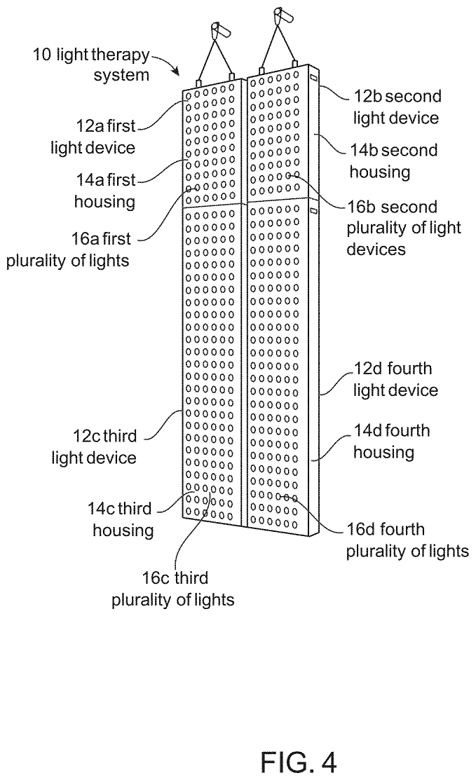

Some treatments may require a larger therapy area than that provided by two light devices. Accordingly, FIG. 4 illustrates a perspective view of an embodiment which comprises, in addition to the first light device 12a and the second light device 12b, a third light device 12c configured to be electrically coupled with the first light device 12a and/or the second device 12b. The third light device 12c may comprise a third housing 14c, a third plurality of lights 16c, and a third power management system 18c (Figures). The embodiment depicted in FIG. 4 also shows a fourth light device 12d configured to be coupled with any one of the first light device 12a, second light device 12b, and/or the third light device 12c. The fourth light device 12d may also comprise a fourth housing 14d, a fourth plurality of lights 16d, and a fourth power management system 18d (Figures). The third plurality of lights 16c and the fourth plurality of lights 16d may also be configured to emit red light, blue light, green light, and/or near infrared light.

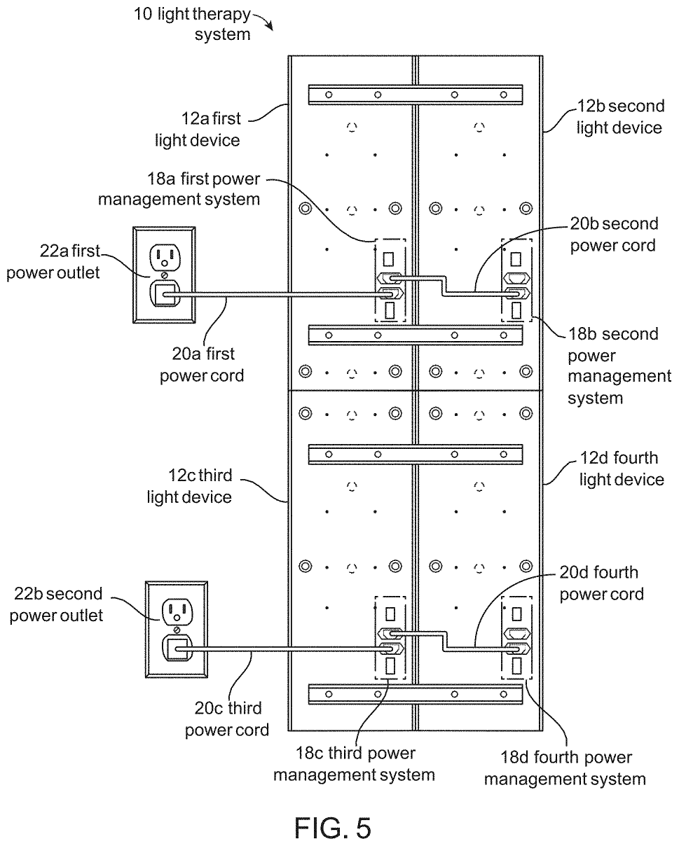

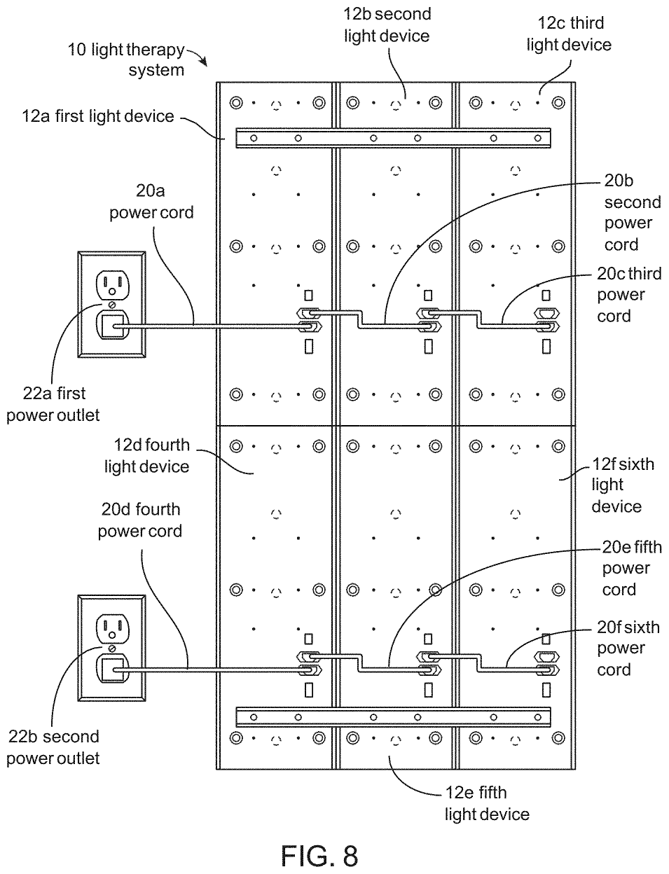

The light therapy system 10 may be arranged in a variety of electrical configurations, whereby light devices 12 are electrically coupled in series. Specifically, FIGS. 5, 6, 8, and 9 illustrate two different scenarios of how the system 10 may be coupled. With respect to FIGS. 5 and 8, the system 10 may be electrically coupled in series having a maximum number of three light devices 12 per series. In other words, three light devices may be electrically coupled in series to a power outlet 22. Because there are four light devices 12 in FIG. 5, the configuration thereby requires two power outlets 22a and 22b. As such, following the discussion with respect to FIG. 3, a third power cord 20c may be electrically coupled to a third power management system 18c and a second power outlet 22b. As well, a fourth power cord 20d may be electrically coupled to the third power management system 18c and a fourth power management system 18d. In this regard, the second and fourth light devices 12b, 12d may thereby receive electrical power from the first and third light devices 12a, 12c, respectively, which in turn receive electrical power from the first and second power outlets 22a, 22b, respectively.

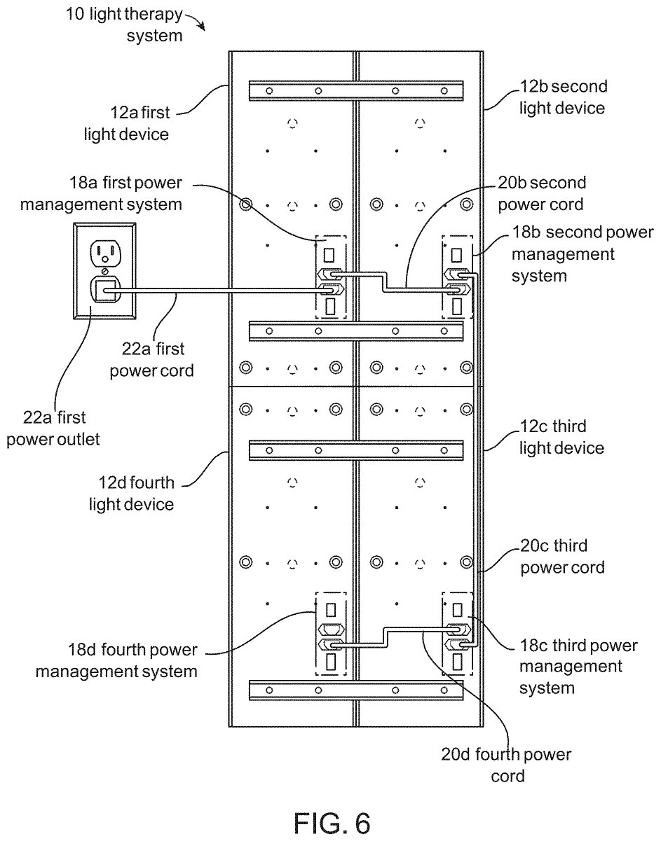

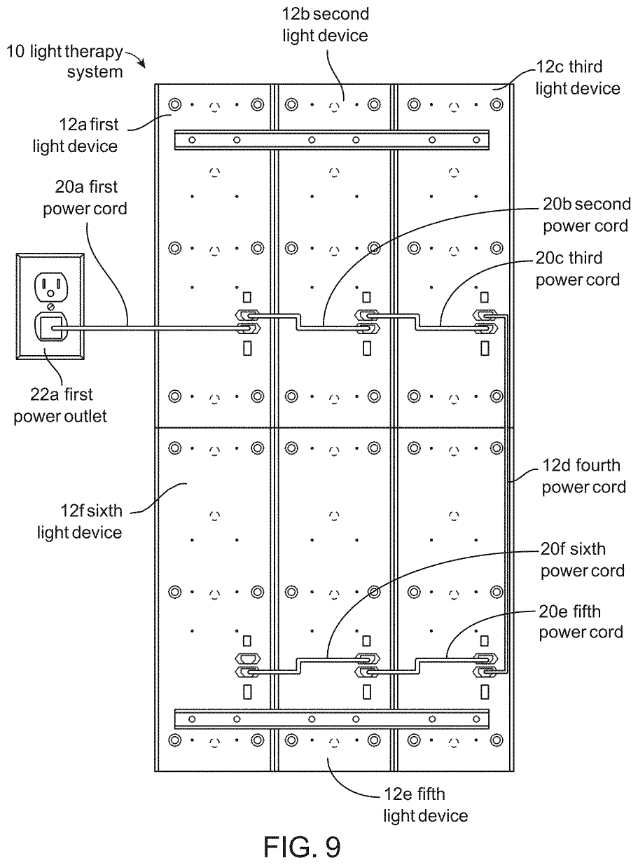

Now, with respect to FIGS. 6 and 9, some embodiments may be configured with no limit on the number of light devices 12 electrically coupled in series. Again, building from the configuration disclosed with respect to FIG. 3, the third power cord 20c may be electrically coupled to the second power management system 18b and the third power management system 18c. The fourth power cord 28d may be electrically coupled to the third power management system 18c and the fourth power management system 18d. Coupling the third power cord in this way removes the need for the second power outlet 22b. This configuration may be advantageous depending on the power requirements of the light therapy system 10. For example, smaller light devices may require less power to achieve the desired outcome. However, a larger light device may require a great deal more power than only one power outlet can provide.

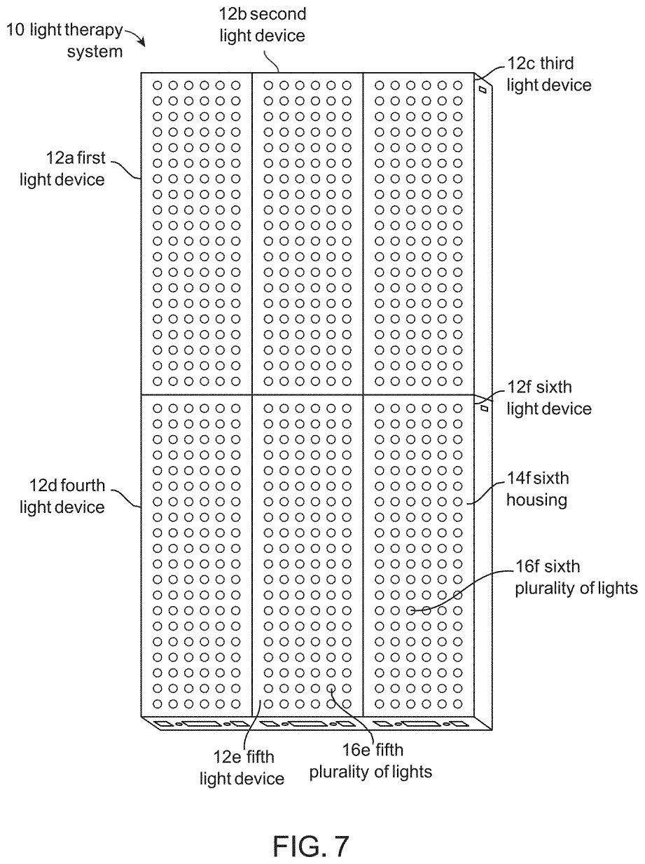

FIGS. 7, 8, and 9 depict how some embodiments of a light therapy system 10 may be further expanded from four to six light devices and how the light devices may be electrically coupled to provide necessary power. As illustrated, a fifth light device 12e may be coupled to the any one of the first, second, third, or fourth light devices 12a, 12b, 12c, 12d. The fifth light device 12e may comprise a fifth housing 14e, a fifth plurality of lights 16e, and a fifth power management system 18e. The fifth power management system 18e may be electrically coupled with the fifth plurality of lights 16e. A sixth light device 12f may be configured to be coupled to any one of the first, second, third, fourth, or fifth light devices 12a, 12b, 12c, 12d, 12e. The sixth light device 12f may comprise a sixth housing 14f, a sixth plurality of lights 16f, and a sixth power management system 18f. The sixth power management system 18f may be electrically coupled with the sixth plurality of lights 16f. It should be appreciated that the plurality of lights 16e and 16f are configured to emit red light, blue light, green light, and/or near infrared light.

Revisiting the manner in which the light devices 12 may be electrically coupled in series we now refer to FIG. 8, which illustrates a light therapy system 10 having six light devices 12a, 12b, 12c, 12d, 12e, and 12f. Because the embodiment illustrated in FIG. 8 is limited to three devices in series, the system 10 thereby requires two power outlets 22a, 22b. Continuing with the discussion from FIG. 5, the fourth power cord 20d may be electrically coupled to the fourth power management system 18d and the second power outlet 22b. A fifth power cord 20e may be electrically coupled to the fourth power management system 18d and the fifth power management system 18e. The sixth power cord 20f may be electrically coupled to the fifth power management system 18e and the sixth power management system 18f. In this embodiment the first through third light devices 12a, 12b, and 12c are connected in a first series, and the fourth through sixth light devices 12d, 12e, and 12f are connected in a second series.

As well, FIG. 9 shows another embodiment in which the light devices 12a, 12b, 12c, 12d, 12e, and 12f are connected in one series. Continuing with the discussion from FIG. 6, the fourth power cord 20d may be electrically coupled to the third power management system 18c and the fourth power management system 18d. The fifth power cord 20e may be electrically coupled to the fourth power management system 18d and the fifth power management system 18e. The sixth power cord 20f may thereby be electrically coupled to the fifth power management system 18e and the sixth power management system 18f. Configuring the light therapy system 10 in this manner avoids the need for the second power outlet 22b.

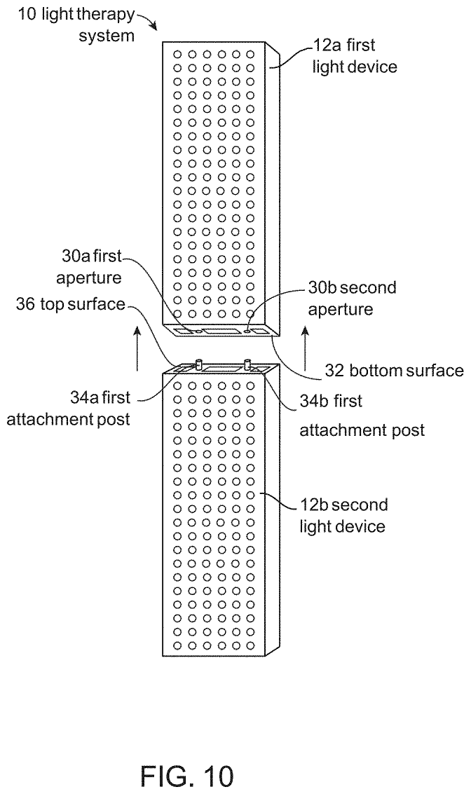

The disclosure also includes embodiments whereby multiple light devices 12 are mechanically coupled together to form a variety of light system sizes and configurations. In doing so, mechanical coupling allows the light devices 12 to work together, as a system, to create one large treatment area. FIG. 10 illustrates an embodiment of how two light devices 12 may be configured for mechanical coupling in a top-to-bottom orientation. The light therapy system 10 of FIG. 10 comprises a first aperture 30a and a second aperture 30b located on the bottom surface 32 of the first housing 14a. A first attachment post 34a and a second attachment post 34b are located on the top surface 36 of the second housing 14b whereby the first and second attachment posts 34a, 34b are spaced from each other. The attachment posts 34 may be thereby securely fit into the apertures 30 to mechanically couple the light devices 12 together. The attachment posts 34 shown in FIG. 10 are posts, but any mechanism suitable for mechanical coupling may be implemented. For example, light devices 12 may be mechanically coupled together via adhesive, bolts, and the like. It should be appreciated that the attachment members 34 are detachably coupled with the apertures 30 such that a user may detach the light devices 12 from one another.

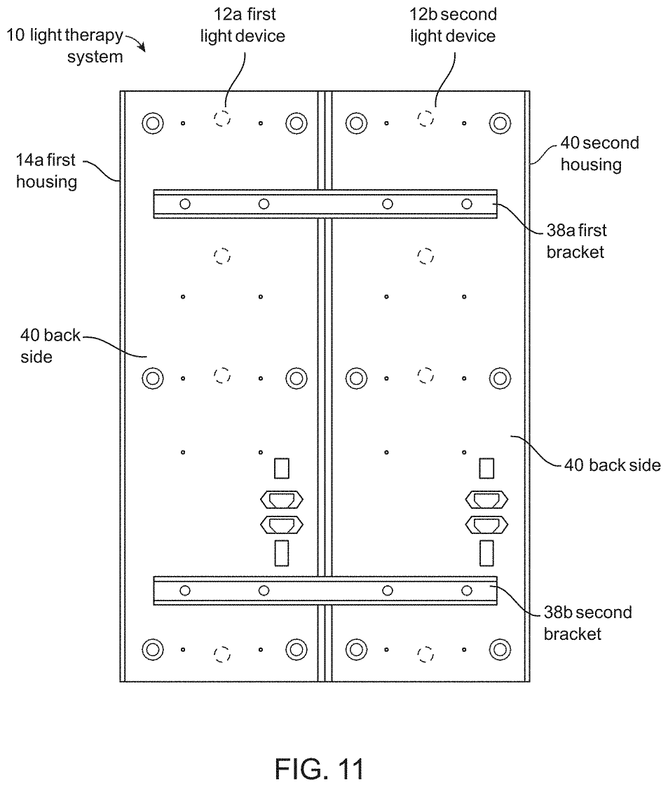

FIG. 11 depicts an embodiment of a light therapy system 10 in which two light devices 12 are mechanically coupled in a side-by-side orientation. The embodiment in FIG. 11 shows a first bracket 38a mechanically coupled to the back side 40 of the first and second light devices 12a and 12b. Similarly, a second bracket 38b is also mechanically coupled to the back side 40 of the first and second light devices 12a and 12b. It should be appreciated that the brackets 38 are detachably coupled to the light devices 12.

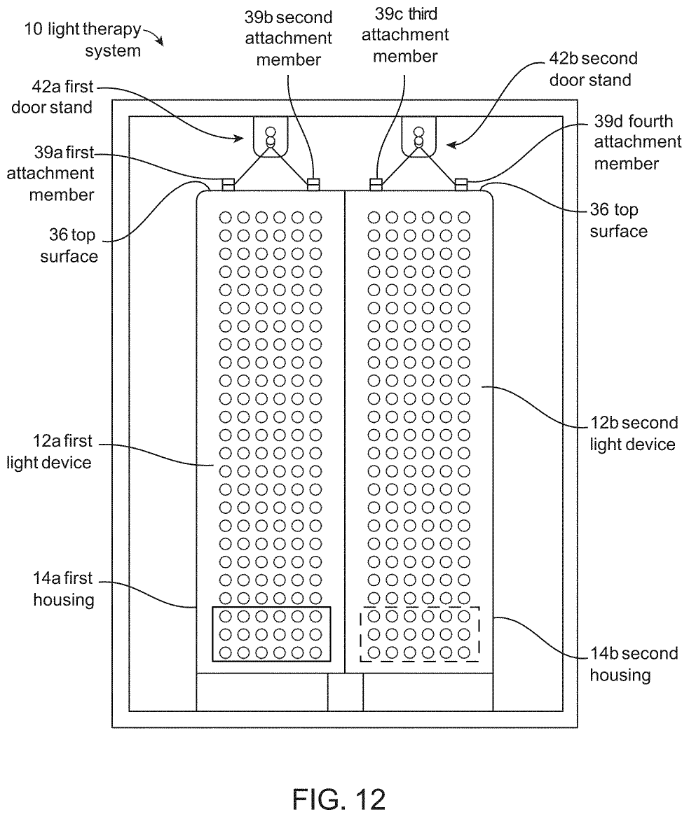

It may be beneficial for the light therapy system 10 to be mounted to an auxiliary support, such as a door stand 42 or a mobile stand 50. This can be achieved in several ways. FIG. 12 shows one embodiment in which two light devices 12a and 12b are mechanically coupled in a side-by-side orientation and mounted to a first and a second door stand 42a and 42b. In doing so, a first attachment member 39a and a second attachment member 39b may extend from the top surface 36 and be detachably coupled to the first door stand 42a. Similarly, the second door stand 42b is detachably coupled to the third attachment member 39c and the fourth attachment member 39d. Moreover, attachment members 39a, 39b, 39c, and 39d may also be referred to as posts. Collectively, one or all of the posts 39a, 39b, 39c, and 39d may be referred to as the attachment system. The attachment system may thereby be configured to detachably couple one or more of the rigid housings to at least one of a door stand and a wall stand. Furthermore, the posts 39a, 39b, 39c, and 39d may be spaced from each other along the top surface of the respective housing.

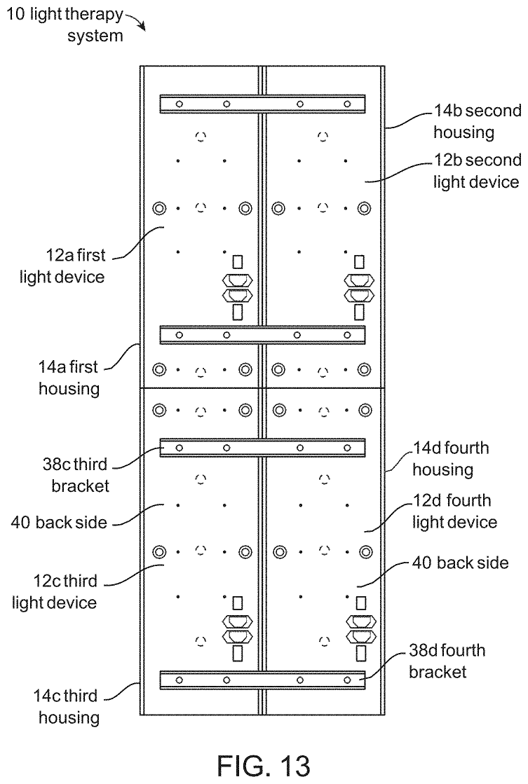

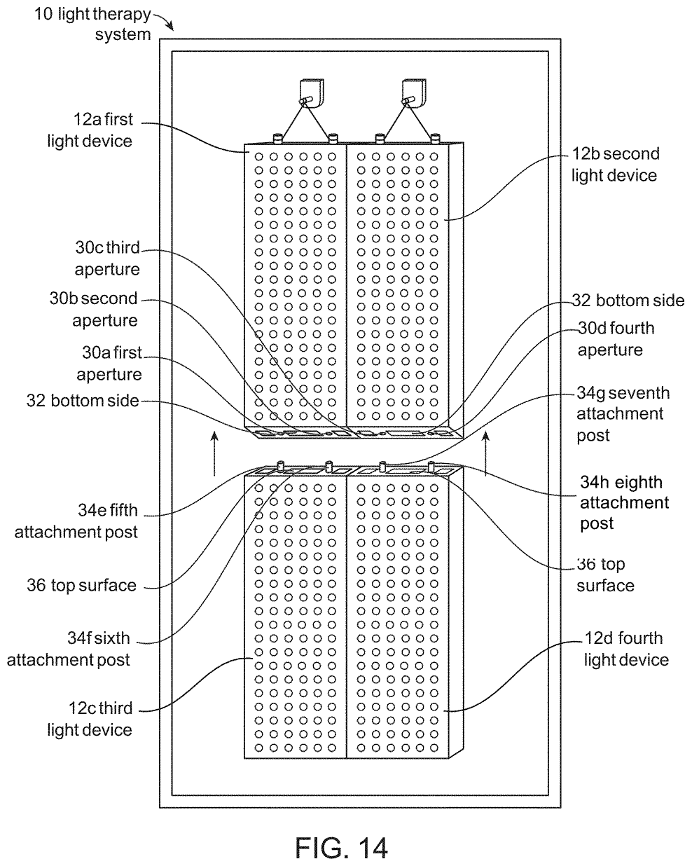

FIGS. 13 and 14 show light therapy system 10 embodiments that combine the top-to-bottom coupling with the side-by-side coupling to achieve a two by two grid of light devices 12. FIG. 13 illustrates a third bracket 38c and a fourth bracket 38d coupled to the back side 40 of the third housing 14c and the fourth housing 14d. FIG. 14 illustrates a fifth attachment post 34e, a sixth attachment post 34f, a seventh attachment post 34g, and an eighth attachment post 34h coupled to the top surface 36 of the third light device 12c and the fourth light device 12d. The illustration in FIG. 14 further depicts the two by two grid of light devices 12 mounted to door stands 42. Collectively, one or all of the attachment posts 34a, 34b, 34c, 34d, 34e, 34f, 34g, and 34h may be referred to as the coupling system. In this regard, the coupling system is configured to detachably couple the first rigid housing 14a to the second rigid housing 14b.

In other embodiments, the four light devices 12 may be mechanically coupled together in other arrangements. For example, one embodiment may have all four light devices 12 coupled together in a side-by-side orientation forming a one by four grid. In other examples, three light devices 12 may be coupled together in a side-by-side orientation and the fourth light device 12d may be coupled to any one of the other three light devices 12 in a top-to-bottom orientation. Another possible embodiment may feature a four by one grid in which the four light devices 12 are all coupled in a top-to-bottom orientation. Generally, it should be appreciated that any arrangement or configuration of light devices may be achieved using the modular concepts disclosed herein.

In order to facilitate ease of using the light therapy system 10 and/or individual light device(s) 12, the disclosure also includes various devices for mounting the system 10 and/or light device(s) 12. For example, users may wish to mount the system 10 and/or device(s) 12 to a stand, such as a mobile stand 50, table top stand 59 (or mini stand 59), wall stand 58, and the like. A stand may thereby allow the system 10 and/or device(s) 12 to be easily transported from one place to another and then quickly configured in a variety of positions to effectively apply therapy to any treatment zone on a patient. Additionally, a stand may allow a user to set up the system 10 and/or light device 12 in an easy to access yet out of the way location, such as the wall stand 58.

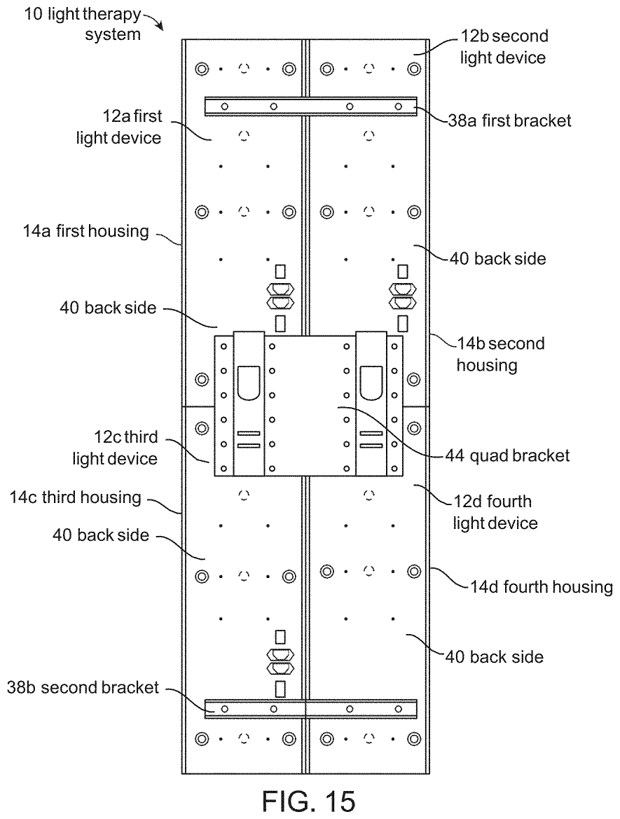

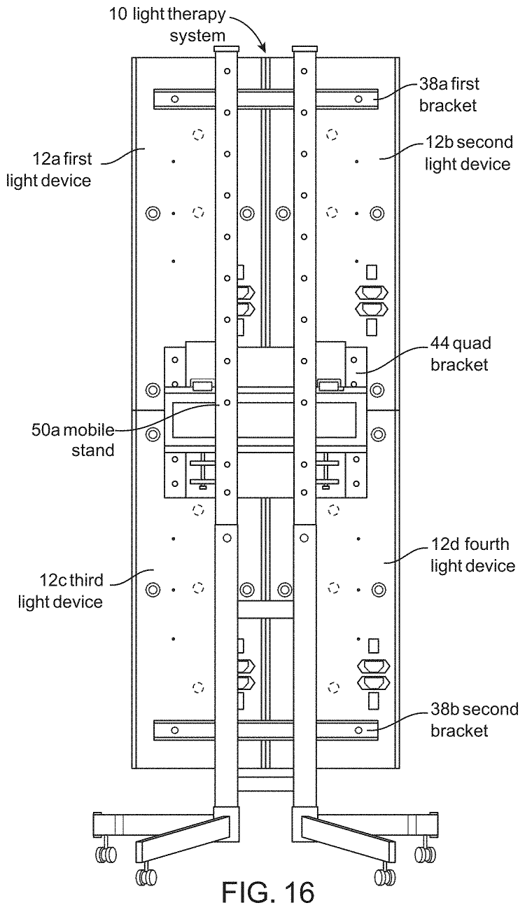

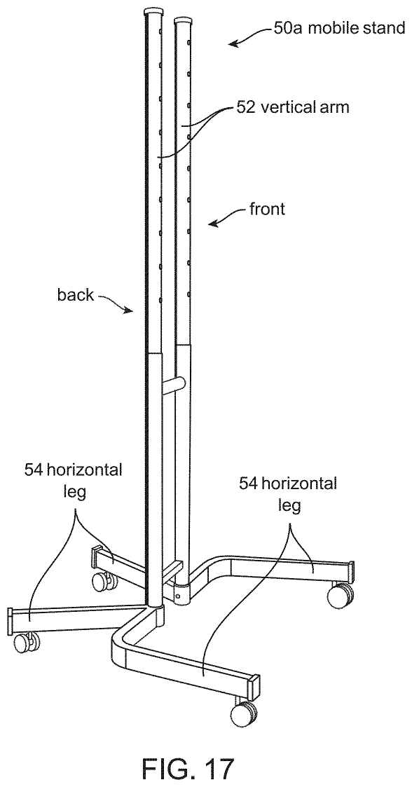

Now with reference to the figures, as shown in FIGS. 15, 16, 17, 18, and 19, the light therapy system 10 includes a mobile stand 50 that couples to one or more brackets on the back side of the various light devices 12. With regard to the four light device embodiment, the first bracket 38a may be coupled the to the back side 40 of the first housing 14a and the second housing 14b and the second bracket 38b may be coupled to the back side 40 of the third housing 14c and the fourth housing 14d. A quad bracket 44 may thereby be coupled to the back side 40 of the first housing 14a, second housing 14b, third housing 14c, and the fourth housing 14d. Accordingly, the mobile stand 50 may be detachably coupled to the light devices 12, the bracket(s) 38, and/or quad bracket 44.

The mobile stand 50a may include one or more horizontal legs 54 to stabilize the system 10. The one or more horizontal legs 54 may include casters to make it easier for a user to move the mobile stand 50a. Casters are not necessary though, for example, wheels may be implemented instead. Even still, the horizontal legs 54 may not include casters or wheels, and the horizontal legs 54 may be affixed to a ground surface or not affixed and free to move with respect to the ground surface. In any case, the horizontal leg 54 may provide balance and stability to the mobile stand 50a. To achieve this, some part of the horizontal leg 54 may contact the ground surface.

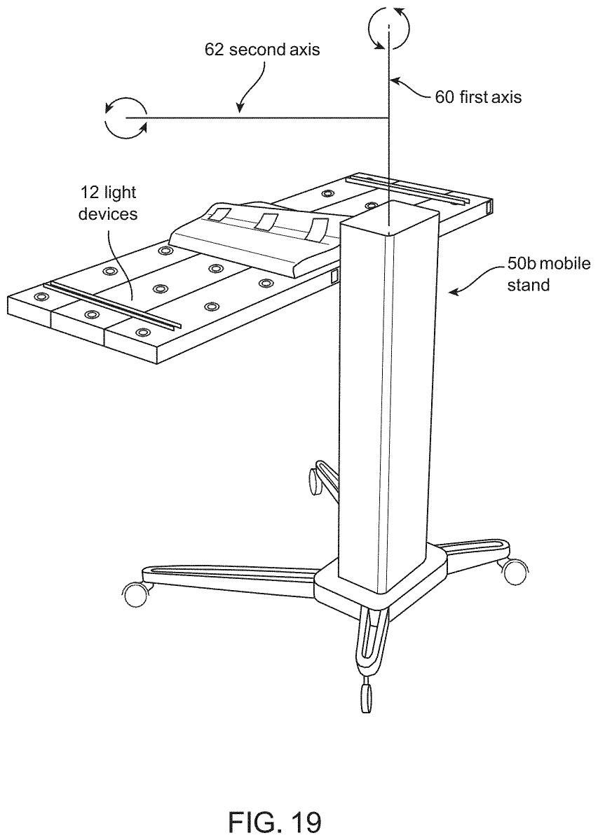

FIG. 19 shows yet another embodiment of a mobile stand 50b. In this embodiment, the mobile stand 50b is configured such that the light devices 12 can be rotated on a first axis 60 and/or a second axis 62. Being able to rotate the light devices 12 along two axes 60, 62 may allow the system 10 to be moved into any possible position to accommodate a desired treatment. For example, if a person is lying down on a flat surface, such as a bed, the light devices 12 can be aimed downwards toward the flat surface, and treatment can be applied to a user's entire front or back side.

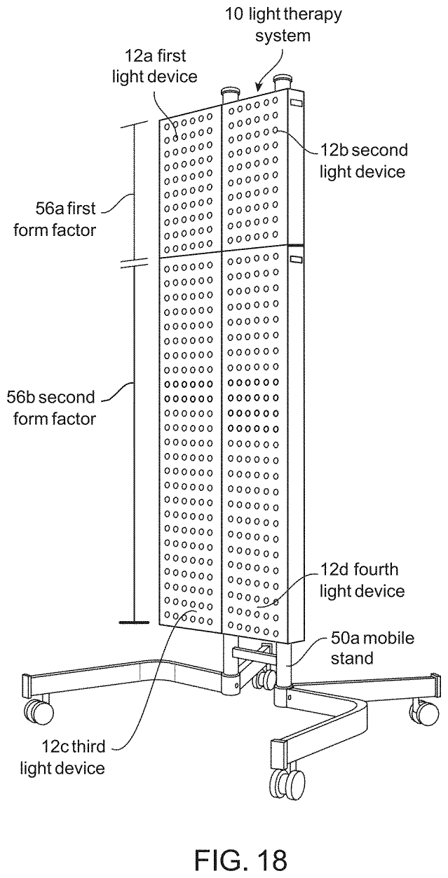

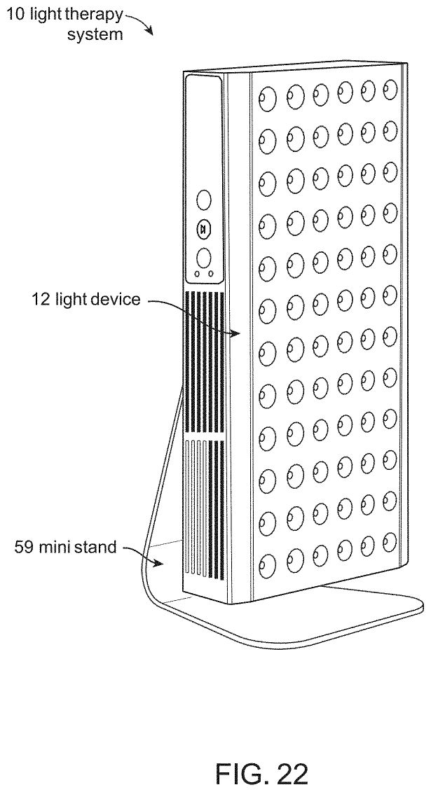

With regards to FIGS. 18 and 22, the light devices 12 may define more than one form factor 56. FIG. 18 shows an embodiment in which the light therapy system 10 is made up of light devices 12 with two different form factors 56a and 56b. The first form factor 56a in FIG. 18 is shorter than the second form factor 56b. Different form factors 56 can be advantageous in situations when a larger treatment area is required than one light device 12 can provide, but there is a lack of space for a second equal size light device 12. Another benefit is that smaller form factors 56a cost less than a larger form factors 56b. In this regard, a user may purchase a smaller light device, such as an entry level device, and then add on to the system 10 once they become familiar and comfortable with the performance of the smaller light device. In some embodiments, the different form factors 56 may be configured to be modular. In this way, any amount of smaller form factor 56a light devices 12 and larger form factor 56b light devices may be mechanically coupled together in either a side-by-side orientation or a top-to-bottom orientation. This allows the light therapy system 10 to be configured into a customizable shape and size for unique treatment areas.

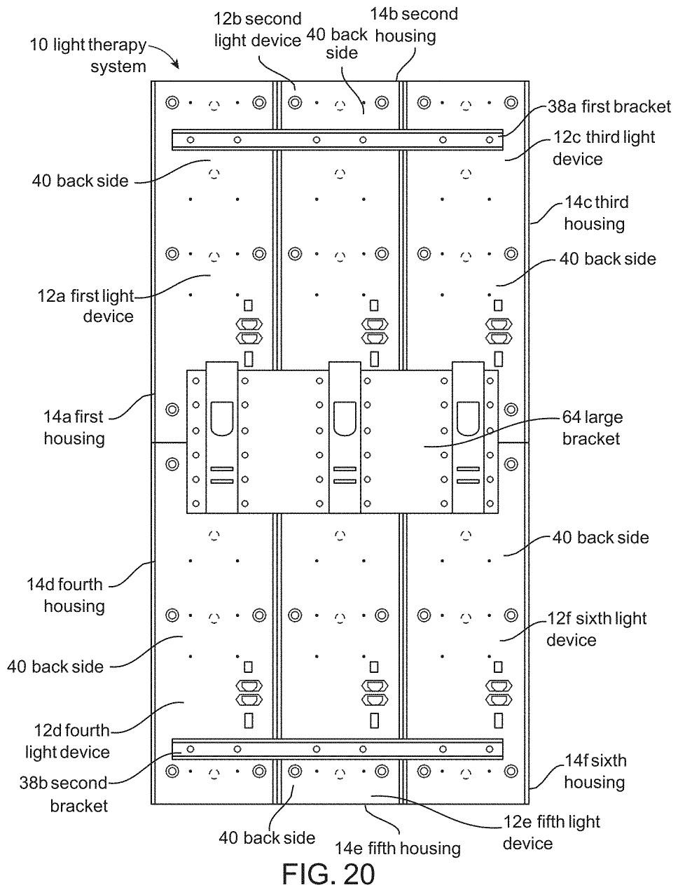

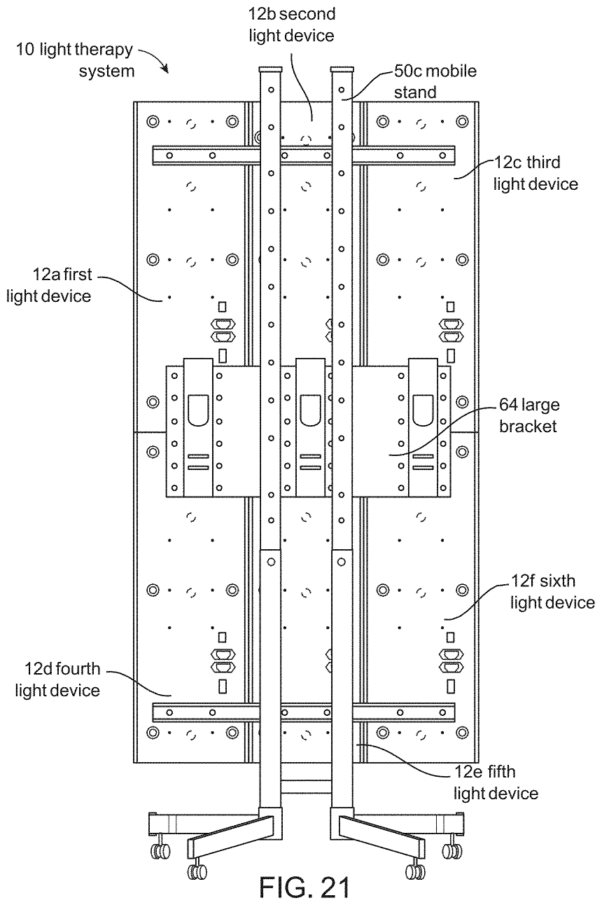

An example of a modular embodiment can be seen in FIG. 20. The illustration shows six light devices 12a, 12b, 12c, 12d, 12e, and 12f mechanically coupled into a two by three grid. Light devices 12a, 12b, and 12c are coupled together via the first bracket 38a, and the light devices 12d, 12e, and 12f are coupled together via the second bracket 38b. As shown, the large bracket 64 located in the middle may mechanically couple all six of the light devices 12 together. FIG. 21 shows an embodiment in which the large bracket 64 also serves as a mount to couple the light system 10 to a mobile stand 50c.

Generally, FIGS. 20 and 21 illustrate the potential for expansion of the system 10. Accordingly, it should be appreciated that any size and shape of brackets 38, 44 may be used to accommodate expansion beyond six light devices 12. In this regard, the system 10 may be configured to have 8, 10, 12, 14, 16, and 24 or more light devices 12 working together as one unified light therapy system 10.

As shown in FIG. 22, the disclosure also includes a single light therapy device that may be arranged and configured with any of the features described herein. As such, a single light therapy device 12 configured to be electrically and/or mechanically coupled to additional light therapy devices. In this regard, the single light therapy device may include any of the features described in the specification and/or shown in the figures.

Furthermore, with continued reference to FIG. 22, the disclosure includes a mini stand 59, which allows the light therapy system and/or one or more light devices (12) to be mounted on a table top, desk, counter, and the like. In this regard, the mini stand 59 may be arranged and configured to couple to a light device 12, such as a smaller light device 12, or plurality of light devices 12 all of which may define smaller form factors sized and configured to sit atop a table top, etc. The mini stand 59 may be able to pivot, raise, and/or lower in any direction with respect to the base of the mini stand 59.

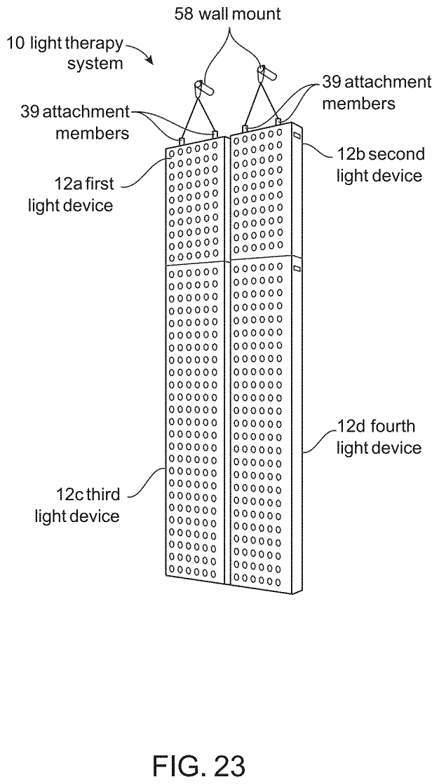

As shown in FIG. 23, the system 10 and/or one or more light devices 12 may also be coupled to a wall mount 58, which can be mounted to a substrate, such as a wall. Specifically, the system 10 may include one or more attachment members 39, or posts 39, located at the top of the respective light device(s) 12. A first portion of the wall mount 58 may thereby be coupled to a wall, while a second portion of the wall mount 58 may be coupled to the attachment member(s) 39 to thereby couple the system 10 and/or light device(s) 12 to the substrate or wall. In this regard, the wall mount 58 may allow the system 10 and/or light device(s) 12 to be mounted in an easy to reach location yet physically situated out of the way to thereby reduce space requirements. Similar to any of the embodiments described throughout, the wall mount 58 may be used to mount one light device 12 or two or more light devices 12.

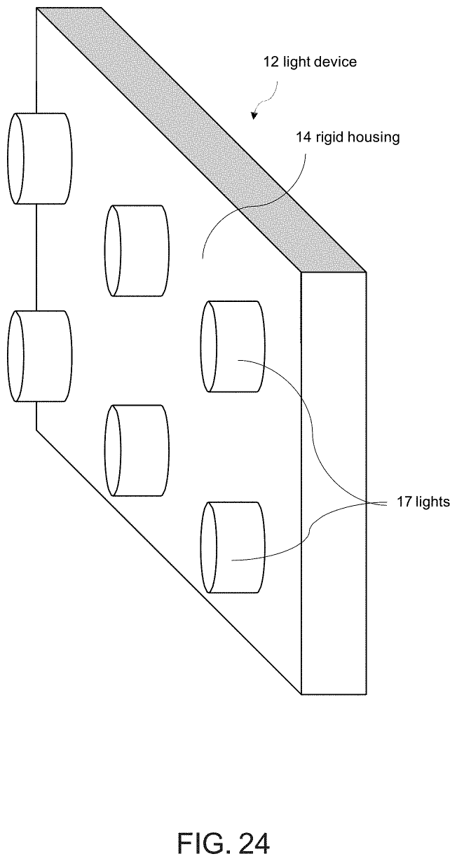

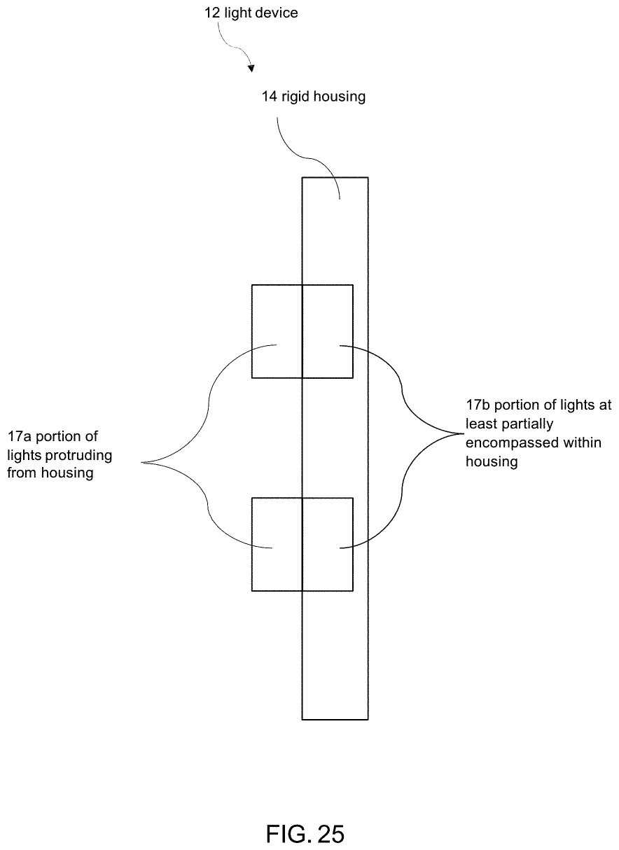

FIGS. 24 and 25 illustrate a partial light therapy device. As shown in FIG. 24, the light therapy device 12 may include a plurality of lights 17 that protrude from the rigid housing 14. In this regard, the light(s) 17 may extend from an interior portion of the rigid housing 14, as shown in FIG. 25, through a light aperture in the rigid housing 14 whereby the light(s) 17 extend away from a surface of the rigid housing 14, as shown in FIG. 24. In this regard, the lights 17 may at least be partially encompassed within the rigid housing and thereby protrude through the rigid housing 14.

### Door Mount System Embodiments

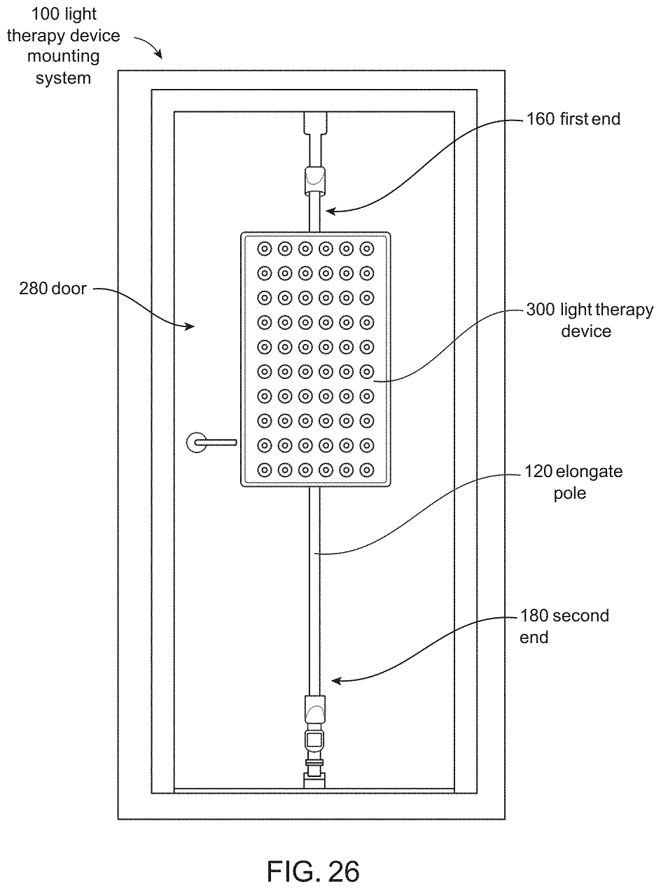

FIG. 26 illustrates an embodiment of a light therapy device mounting system 100. As shown in the Figure, the system 100 may be configured to couple to a door 280. In many embodiments, the system 100 includes an elongate pole 120 comprising a first end 160 and a second end 180. The first end 160 may be considered the “top” (or adjacent the top) of the elongate pole 120, and the second end 180 may be considered the “bottom” (or adjacent the bottom) of the elongate pole 120. A light therapy device 300 may be configured to couple to the elongate pole 120 between the first end 160 and the second end 180. The manner in which the light therapy device 300 couples to the elongate pole 120 will be discussed in greater detail later in the disclosure. Though a single light therapy device 300 is shown coupled to the elongate pole 120, multiple light therapy devices may couple to the elongate pole 120. In some embodiments, the light therapy device 300 comprises a larger device 300 than depicted in FIG. 26. The light therapy device 300 may comprise a smaller device 300 than depicted in FIG. 26. The system 100 may be configured to include a plurality of elongate poles 120 coupled to the door 280, and each elongate pole 120 may be configured to couple to at least one light therapy device 300.

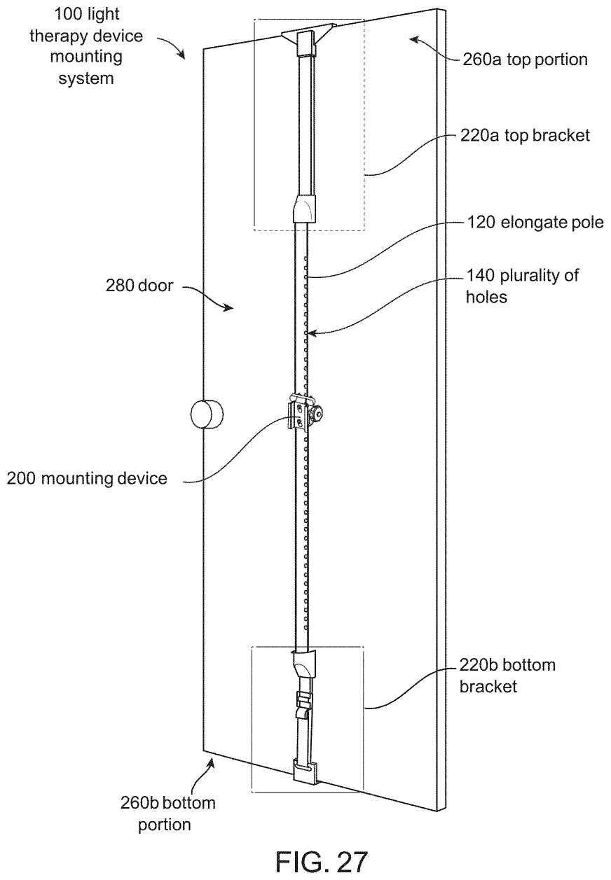

FIG. 27 illustrates a perspective view of the system 100 coupled to the door 280. FIG. 27 is similar to FIG. 26, but shows the system 100 without the light therapy device 300 and illustrates additional elements of the system 100. For example, FIG. 27 includes the mounting device 200, which will be discussed in greater detail later in the disclosure, in particular with reference to FIGS. 26 and 27. FIG. 27 also demonstrates that, in many embodiments, the elongate pole 120 comprises a plurality of holes 140. The plurality of holes 140 may extend in a substantially straight line down a side portion of the pole 120, as illustrated in FIG. 27. The plurality of holes 140 may extend down substantially the full length of the pole 120. As shown in FIG. 27, the plurality of holes 140 may extend down less than the full length of the pole 120. In some embodiments, the plurality of holes 140 comprises more than one line of holes. The plurality of holes 140 may be featured in a scattered pattern around the pole 120. The plurality of holes 140 may be located anywhere on the surface of the pole 120. In some embodiments, the plurality of holes 140 is configured to accommodate height adjustment of the placement of the mounting device 200, and thereby, of a light therapy device 300 coupled to the mounting device 200.

FIG. 27 also includes a top bracket 220a and a bottom bracket 220b. In many embodiments, the top bracket 220a is coupled to the first end 160 of the elongate pole 120, and the bottom bracket 220b is coupled to the second end 180 of the elongate pole 120. The brackets 220a, 220b may be slideably coupled to the elongate pole 120 at their respective ends 160, 180, wherein the brackets 220a, 220b may be configured to slideably receive the ends 160, 180. In some embodiments, the brackets 220a, 220b are coupled in a different manner (e.g., snapably, mechanically, threadably, via a friction fit, via a mechanism such as a screw or locking pin, via the plurality of holes 140, and the like). The brackets 220a, 220b may be detachably coupled to the elongate pole 120. The top bracket 220a may also be configured to detachably couple to a top portion 260a of the door 280, and the bottom bracket 220b may be configured to detachably couple to a bottom portion 260b of the door 280.

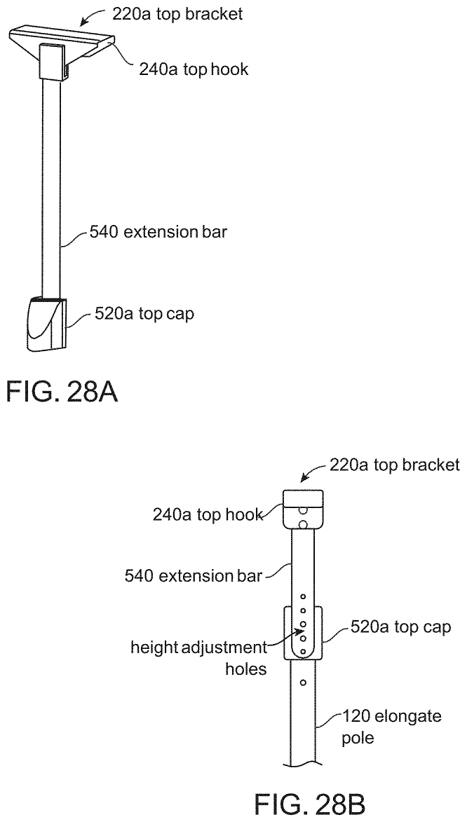

The top bracket 220a is shown in greater detail in FIGS. 28A and 28B, which show front and back views, respectively, of an embodiment of the top bracket 220a. In many embodiments, the top bracket 220a comprises a top cap 520a, a top hook 240a, and an extension bar 540. As previously stated, the top bracket 220a may be slideably coupled to a first end 160 of the elongate pole 120. In some embodiments, the top bracket 220a is slideably coupled via the top cap 520a, which is configured to receive the elongate pole 120. The top cap 520a may include two pieces configured to snap together around the elongate pole 120. In some embodiments, one-piece (e.g., a back piece) of the top cap 520a is configured to receive the elongate pole 120, and the second piece (e.g., a front piece) is configured to cover the first end 160 and the first piece of the cap 520a, thus providing a cosmetic appearance to the top cap 520a. In some embodiments, the top cap 520a comprises a single piece configured to substantially surround the first end 160 of the elongate pole 120. The top cap 520a may comprise more than two pieces.

In some embodiments, the top cap 520a is configured to couple to the extension bar 540. As shown in FIG. 28B, the extension bar 540 may include at least one height adjustment hole. In many embodiments, the at least one height adjustment hole is configured to couple to the top cap 520a via at least one coupling mechanism, such as a bolt. The at least one coupling mechanism may be located on a back portion of the top cap 520a, where a back portion may be considered a portion opposite a portion of the system 100 coupled to the light therapy device 300. For example, in the embodiment shown in FIG. 26, the system 100 is configured such that the light therapy device 300 faces away from the door 280. In such an embodiment, the back portion of the top cap 520a may be considered the portion located adjacent the door 280. In some embodiments, the at least one height adjustment hole and the at least one coupling mechanism are located within the top cap 520a. The at least one height adjustment hole and the at least one coupling mechanism may be located on a front portion located opposite the back portion of the top cap 520a.

The at least one height adjustment hole may be configured to receive the at least one coupling mechanism such that if the top height adjustment hole receives the at least one coupling mechanism, the extension bar 540 is configured to sit at a minimum height. If the bottom height adjustment hole receives the at least one coupling mechanism, the extension bar 540 may be configured to sit at a maximum height. In many embodiments, the extension bar 540 is configured to accommodate a range of door heights by raising or lowering the height of the top hook 240a. The extension bar 540 may be configured to accommodate door heights of about 80 inches. The extension bar 540 may be configured to accommodate doors shorter than 80 inches. In some embodiments, the extension bar 540 is configured to accommodate doors taller than 80 inches. Embodiments of the extension bar 540 may define different lengths in order to accommodate different door heights. For example, in some embodiments, the extension bar 540 measures about 9.5 inches long, which may be used for a door about 80 inches tall. The extension bar 540 may measure about 25 inches long, which may be used for a door taller than 80 inches. The extension bar 540 may measure any length between about 9.5 inches and about 25 inches in length. The extension bar 540 may measure shorter than 9.5 inches, which may be used for a door shorter than 80 inches. In some embodiments, the extension bar 540 measures longer than 25 inches. Such embodiments may enable the system 100 to couple to an oversize door significantly taller than 80 inches.

As shown in FIG. 27, the top hook 240a may be configured to receive a top portion 260a of the door 280. The top hook 240a may define a range of widths, as demonstrated by FIGS. 28A and 28B. In many embodiments, the top hook 240a is configured to receive the top portion 260a of the door 280 such that the top hook 240a extends across a top edge of the door 280 and at least partially down a side of the door 280 located opposite the side of the door 280 adjacent the light therapy device 300. For example, if the light therapy device 300 is coupled to a side of the door 280 facing into a room when the door 280 is in a closed position, the top hook 240a may extend at least partially down the side of the door 280 facing out into a hallway. In many embodiments, the top hook 240a is sized and configured to not impede operation of the door 280, such that the door 280 may be moved between an open position and a closed position (and vice versa) when the system 10, including the top hook 240a, is coupled to the door 280. The top hook 240a may be sized and configured to produce minimal contact with a door jamb when the door 280 is in the closed position. For example, the top hook 240a may define a thickness of a few millimeters where “thickness” is defined as the amount of the top hook 240a that sits on top of the top edge of the door 280, between the top edge and the door jamb.

The top hook 240a may be coupled to the extension bar 540 in a number of suitable ways, including but not limited to, slideably coupled, mechanically detachably coupled via a mechanism such as a bolt, screw, etc., and coupled via welding, adhesive, and the like. In some embodiments, the top hook 240a is fixedly coupled to the extension bar 540 via a plurality of fasteners with locking nuts. The fasteners may comprise 4mm fasteners. In some embodiments, the extension bar 540 is coupled to a back portion of the top hook 240a located adjacent the door 280. The extension bar 540 may be coupled to a front portion of the top hook 240a, where the front portion is located opposite the back portion.

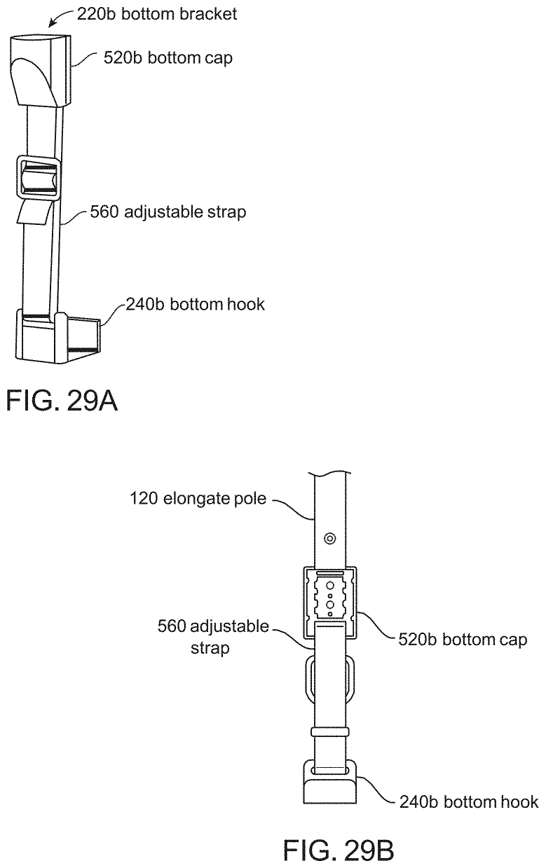

FIGS. 29A and 29B are similar to FIGS. 28A and 28B; however, they illustrate the bottom bracket 220b rather than the top bracket 220a. As shown, in many embodiments, the bottom bracket 220b includes a bottom cap 520b, and adjustable strap 560, and a bottom hook 240b. The bottom cap 520b may be configured to slideably couple the bottom bracket 220b to the second end 180 of the elongate pole 120. In some embodiments, the bottom cap 520b is configured to slideably receive the elongate pole 120. The bottom cap 520b may include two pieces configured to snap together around the elongate pole 120. In some embodiments, one-piece (e.g., a back piece) of the bottom cap 520b is configured to receive the elongate pole 120, and the second piece (e.g., a front piece) is configured to cover the second end 180 and the first piece of the cap 520b, thus providing a cosmetic appearance to the bottom cap 520b. In some embodiments, the bottom cap 520b comprises a single piece configured to substantially surround the second end 180 of the elongate pole 120. The bottom cap 520b may comprise more than two pieces. The bottom cap 520b may be configured to store the excess length of the adjustable strap 560 after the strap 560 is tightened and/or loosened.

In some embodiments, the bottom cap 520b is configured to couple to the adjustable strap 560. The adjustable strap 560 may loop around a portion of the bottom cap 520b. In some embodiments, the portion comprises at least one of a back piece, a front piece, and an internal piece of the bottom cap 520b. The portion may comprise a horizontal bar or similar portion. The adjustable strap 560 may couple to the bottom cap 520b in any other suitable way. In many embodiments, the adjustable strap 560 comprises a flexible strap and an adjusting mechanism. The adjusting mechanism may comprise a strap adjuster, a buckle, a slider, a cord lock, or any similar adjusting mechanism. In some embodiments, the adjusting mechanism is configured to at least one of tighten and loosen the adjustable strap 560. Tightening the adjustable strap 560 may facilitate tightening the system 100 by increasing the tension between the bottom hook 240b and the bottom cap 520b, and, therefore, between the bottom hook 240b and the elongate pole 120. Tightening the system 100 may be desirable in order to reduce the amount of movement of the system 100 on the door 280, particularly when the door 280 is moved between a closed position and an open position, and vice versa. Loosening the adjustable strap 560 may reduce the tension between the bottom hook 240b and the bottom cap 520b, and, therefore, between the bottom hook 240b and the elongate pole 120, thereby facilitating loosening of the system 100 so that the system 100 may be removed from and/or adjusted on the door 280. In some embodiments, the system 100 is arranged such that the top hook 240a is configured to couple to the door 280, then the bottom hook 240b is configured to couple to the door 280, then the adjustable strap 560 is configured to tighten.

As shown in FIG. 27, the bottom hook 240b may be configured to receive a bottom portion 260b of the door 280. In some embodiments, the bottom hook 240b defines a different width than the top hook 240a. The bottom hook 240b may define substantially the same width as at least one embodiment of the top hook 240a. In many embodiments, the bottom hook 240b is configured to receive the bottom portion 260b of the door 280 such that the bottom hook 240b extends across a bottom edge of the door 280 and at least partially up a side of the door 280 located opposite the side of the door 280 including the light therapy device 300. For example, if the light therapy device 300 is coupled to a side of the door 280 facing into a room when the door 280 is in a closed position, the bottom hook 240b may extend at least partially up the side of the door 280 facing out into a hallway. The bottom hook 240b may extend up the opposite side of the door 280 substantially the same amount as the top hook 240a extends down the opposite side of the door 280. The top hook 240a and bottom hook 240a may extend different amounts down and up, respectively, the opposite side of the door 280.

In many embodiments, the bottom hook 240b is sized and configured to not impede operation of the door 280, such that the door 280 may be moved between an open position and a closed position (and vice versa) when the system 100, including the bottom hook 240b, is coupled to the door 280. The bottom hook 240b may be sized and configured to produce minimal contact with a floor surface. For example, the bottom hook 240b may define a thickness of a few millimeters where “thickness” is defined as the amount of the bottom hook 240b that sits under the bottom edge of the door 280, between the bottom edge and the floor surface. In many embodiments, the top hook 240a and the bottom hook 240b define substantially the same thickness. The top hook 240a and the bottom hook 240b may define different thicknesses.

In some embodiments, the adjustable strap 560 is configured to couple to the bottom hook 240b by looping around a portion of the hook 240b, as shown in FIGS. 29A and 29B. The adjustable strap 560 may couple to the bottom hook 240b in other ways, including fixed coupling. In some embodiments, the adjustable strap 560 is fixedly coupled to the bottom hook 240b and movably coupled to the bottom cap 520b. The adjustable strap 560 may be movably coupled to the bottom hook 240b and fixedly coupled to the bottom cap 520b. The adjustable strap 560 may be movably coupled to both the bottom cap 520b and the bottom hook 240b. The adjustable strap 560 may be fixedly coupled to both the bottom cap 520b and the bottom hook 240b.

In many embodiments, at least one of the top hook 240a and the bottom hook 240b is sized and configured to fit a standard door depth. An example of a standard door depth is about 1.375 inches. As such, in some embodiments, at least one of the top hook 240a and the bottom hook 240b defines a depth of about 1.375 inches. At least one of the top hook 240a and the bottom hook 240b may be sized slightly larger than a standard door depth to accommodate deeper doors. In some embodiments, at least one of the top hook 240a and the bottom hook 240b defines a depth of about 1.875 inches. At least one of the top hook 240a and the bottom hook 240b may define a depth of greater than 1.875 inches. At least one of the top hook 240a and the bottom hook 240b may define a depth of less than 1.375 inches. In many embodiments, the top hook 240a and the bottom hook 240b define substantially the same depth. The top hook 240a and the bottom hook 240b may define different depths.

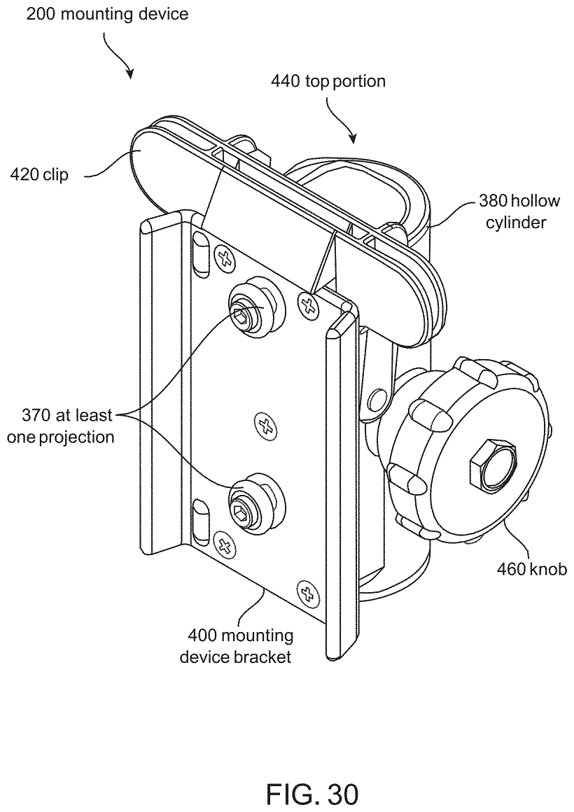

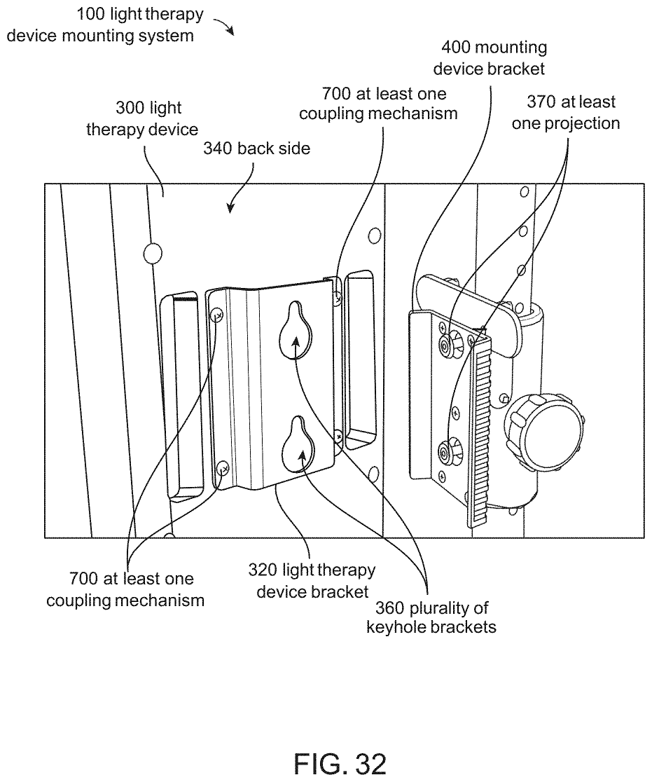

FIG. 30 shows a perspective view of a mounting device 200. In many embodiments, the mounting device 200 includes a hollow cylinder 380, a mounting device bracket 400, a clip 420, and a knob 460. The mounting device 200 may also include at least one projection 370 coupled to a front-facing surface of the mounting device bracket 400, which will be discussed in greater detail with reference to FIG. 32. In some embodiments, the clip 420 is located at a top portion 440 of the mounting device 200. The clip 420 may be located at a bottom portion opposite the top portion. The knob 460 may be coupled to a side portion of the hollow cylinder 380. The mounting device bracket 400 may be configured to couple to the at least one of the clip 420 and the hollow cylinder 380 via any of the coupling mechanisms disclosed herein.

The hollow cylinder 380 may be configured to slideably receive the elongate pole 120, as demonstrated in FIG. 27. At least one of a top end and a bottom end of the hollow cylinder 380 may comprise a collar configured to at least one of substantially surround and cover a top edge and bottom edge, respectively, of the cylinder 380. In many embodiments, the mounting device 200 is configured to couple to the elongate pole 120 via at least one hole of the plurality of holes 140, shown in FIG. 27. It should be noted that FIG. 27 shows the mounting device 200 without the mounting device bracket 400 or the clip 420.

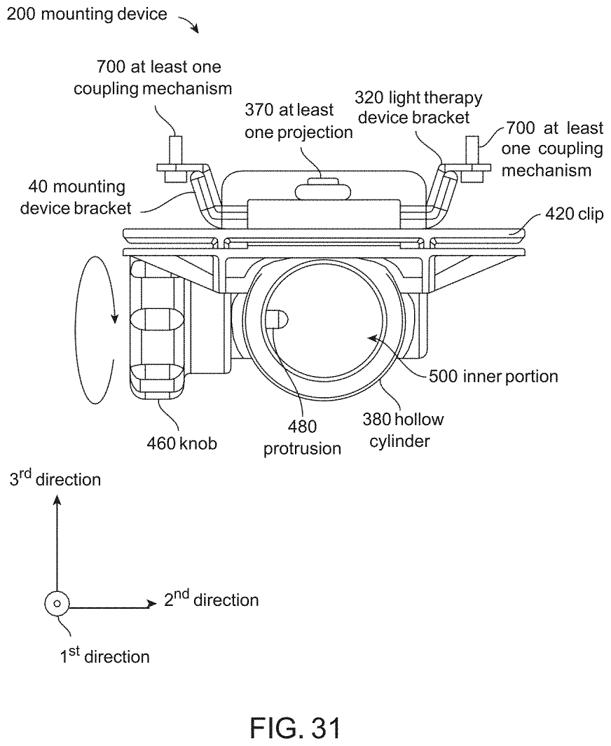

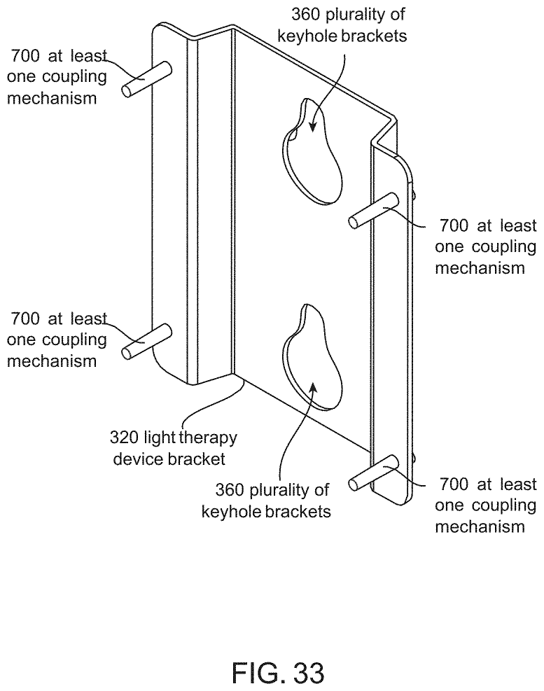

FIG. 31 shows a top view of the mounting device 200 and includes the light therapy device bracket 320, which will be discussed further with reference to FIGS. 32 and 33. In many embodiments, the elongate pole 120 (not shown in FIG. 31) extends along a first direction. The knob 460 may be configured to slide along a second direction, where the second direction may be perpendicular to the first direction, as shown by the directional indicators included in FIG. 31. In many embodiments, when the knob 460 slides along the second direction, a protrusion 480 coupled to the knob 460 at least one of extends into and retracts from an inner portion 500 of the hollow cylinder 380. At least one hole of the plurality of holes 140 of the elongate pole 120 may be configured to receive the protrusion 480, thereby coupling the mounting device 200 to the elongate pole 120. Stated another way, the protrusion 480 may operate similarly to a locking pin. In some embodiments, the knob 460 is pulled along the second direction away from the hollow cylinder 380, thereby retracting the protrusion 480 and allowing the mounting device 200 to slide on the elongate pole 120 in the first direction. Once the mounting device 200 is in a desired location (i.e., at a desired height) on the elongate pole 120, the knob 460 may be pushed toward the hollow cylinder 380, thereby inserting the protrusion 480 into the inner portion 500 and coupling the protrusion 480 to at least one hole of the plurality of holes 140. The knob 460 may include a plurality of protrusions 48 configured to couple to the plurality of holes 140. The knob 460 may comprise a spring mechanism configured to enable retraction of the protrusion 480.

In many embodiments, the knob 460 is configured to rotate along a third direction, where the third direction is perpendicular to both the first direction and the second direction. Rotating the knob 460 may be configured to tighten and/or loosen the knob 460, thereby tightening and/or loosening the coupling between the hollow cylinder 380 and the elongate pole 120. For example, after coupling the mounting device 200 to the elongate pole 120 via the protrusion 480 and the plurality of holes 140, a user of the system 100 may desire to tighten the knob 460 in order to provide greater security to the location of the mounting device 200 on the pole 120. Prior to retracting the protrusion 480 to move the mounting device 200, the user may then rotate the knob 460 in order to loosen the knob 460 before sliding the knob 460 along the second direction.

FIG. 32 shows a perspective view of the system 100, including a back side 340 of a light therapy device 300. As shown, in many embodiments, the light therapy device 300 includes a light therapy device bracket 320 located on the back side 340 of the light therapy device 300. The light therapy device bracket 320 may include a plurality of keyhole brackets 360 configured to detachably couple to the at least one projection 370, thereby detachably coupling the light therapy device bracket 320 to the mounting device bracket 400. In some embodiments, the at least one projection 370 is coupled to a front face of the mounting device bracket 400. The at least one projection 370 may be configured to extend through the bracket 400 to the front face from a back portion located adjacent the hollow cylinder 380. The at least one projection 370 may be coupled to the hollow cylinder 380. In some embodiments, the light therapy device bracket 320 is coupled to the back side 340 of the light therapy device 300 via at least one coupling mechanism 700. The at least one coupling mechanism 700 may include any suitable mechanism, including but not limited to, screw(s), bolt(s), staple(s), magnet(s), adhesive, welding, and the like. FIG. 33 shows a standalone view of the light therapy device bracket 320. Though shown in FIGS. 32 and 33 with four mechanisms in the at least one coupling mechanism 700, it should be noted that the bracket 320 may include fewer or more than four coupling mechanisms 700. The bracket 320 also may include a single keyhole bracket. In some embodiments, the plurality of keyhole brackets 360 comprises more than two keyhole brackets 360.

The clip 420 shown in FIGS. 30 and 31 may be configured to retain the light therapy device bracket 320 in a locked position on the mounting device bracket 400. In some embodiments, when the light therapy device bracket 320 is coupled to the mounting device bracket 400, a top edge of the light therapy device bracket 320 is configured to sit below the clip 420. As such, the clip 420 may move back toward the elongate pole 120 to allow the bracket 320 to slide onto the bracket 400, then may “spring” back into the original position, thereby retaining the bracket 320 on the bracket 400. The clip 420, and the extra security it provides, may enable the system 100 to withstand a sudden movement (e.g., person bumps into system 100, door 280 slammed into a wall, earthquake, and the like) and prevent the light therapy device 300 from being dislodged from the mounting device bracket 400. In many embodiments, the clip 420 is a spring-loaded clip. The clip 420 may be any suitable type of clip.

In many embodiments, to release the light therapy device 300, a user moves the clip 420 back toward the elongate pole 120 and raises the light therapy device bracket 320 (and light therapy device 300) up and off of the mounting device bracket 400. The movements of the clip 420 may be the inverse of what has been disclosed. For example, the clip 420 may move toward the light therapy device 300 in order to release and toward the elongate pole 120 in order to engage the light therapy device bracket 320. In some embodiments, the system 100 includes a plurality of clips 420 on the mounting device bracket 400.

### Mobile Stand System Embodiments

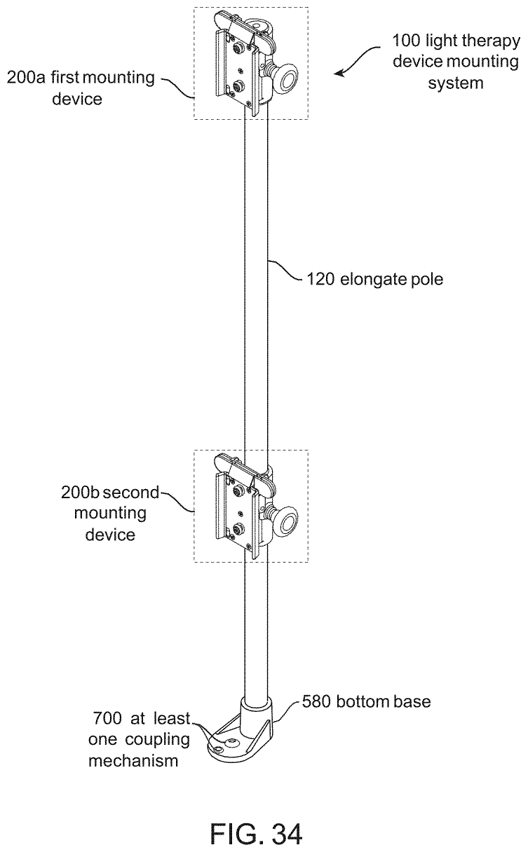

FIG. 34 shows an embodiment of the light therapy device mounting system 100. Like the embodiments shown in FIGS. 22 and 23, the system 100 illustrated in FIG. 34 includes an elongate pole 120 and a mounting device 200. Though not shown in FIG. 34, the elongate pole 120 may include a plurality of holes 140, and the mounting device 200 may be configured to couple to the elongate pole 120 via at least one hole of the plurality of holes 140, as discussed with reference to FIG. 31. The elongate pole 120 may also comprise a first end 160 and a second end 180. In some embodiments, the system 100 includes a first mounting device 200a and a second mounting device 200b. The first mounting device 200a may be coupled to a light therapy device 300, and the second mounting device 200b may be coupled to another light therapy device 300. As such, multiple light therapy devices 300 may be coupled to the elongate pole 120 simultaneously.

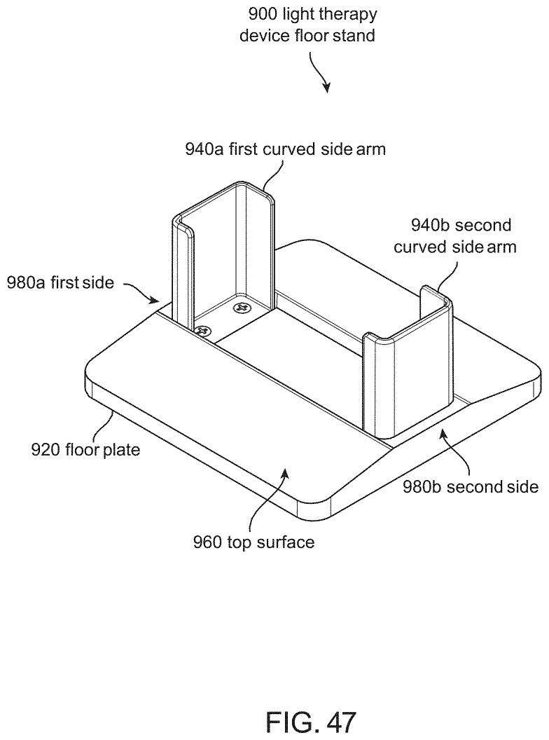

FIG. 34 also illustrates that, in some embodiments, the system 100 includes a bottom base 580 coupled to the elongate pole 120. The bottom base 580 may be configured to slideably receive the elongate pole 120. Other methods of coupling, including, but not limited to, a friction fit, threadably coupling, coupling via gravity, and mechanical coupling may be suitable to couple the elongate pole 120 to the bottom base 580. The bottom base 580 may be configured to couple to the elongate pole 120 at the second end 180 (shown in FIG. 47) of the elongate pole 120. As illustrated, the bottom base 580 may be shaped similar to an elbow connector. The bottom base 580 may define any suitable shape to receive the elongate pole 120 and couple to a stand. In many embodiments, the bottom base 580 is configured to support the elongate pole 120 and any other elements (e.g., mounting device 200, light therapy device 300, and the like coupled to the elongate pole 120) in an upright position.

In some embodiments, the system 100 includes an additional element, not shown in the Figures, configured to facilitate coupling between the bottom base 580 and the elongate pole 120. The additional element may comprise a plug-type component configured to slideably couple to the bottom base 580. For example, the bottom base 580 may be configured to receive the plug and couple to the plug via at least one mechanical coupling mechanism. The elongate pole 120 may then be configured to slide into the bottom base 580 around the plug, such that when the elongate pole 120 is coupled to the bottom base 580, the plug is located at least partially within an interior portion of the elongate pole 120. The bottom base 580 and the plug may comprise at least one hole located on a bottom and/or back surface, wherein the at least one hole is configured to receive a screw, bolt, or similar device to mechanically couple the plug to the bottom base 580. The plug may be configured to provide additional support and stability to the elongate pole 120 when the elongate pole 120 is coupled to the bottom base 580.

In some embodiments, the elongate pole 120 is configured for use with both the door mount embodiment shown in FIGS. 22 and 23 and the mobile stand embodiments discussed herein. As previously discussed, the elongate pole 120 may be detachably coupled to the top bracket 220a and the bottom bracket 220b of the system 100. Once decoupled from the bottom bracket 220b, the elongate pole 120 may be configured to couple to the bottom base 580. The bottom base 580 may be configured to couple to a variety of stands, as will be discussed in further detail with reference to FIGS. 31-42. As such, a user of the system 100 may obtain a single elongate pole 120 to facilitate use of a light therapy device 300 on a door mount, a mobile stand, and/or a fixed stand. Stated differently, the elongate pole 120 may be interchangeably coupled to a variety of components (e.g., top bracket 220a, bottom bracket 220b, bottom base 580) in order to facilitate a variety of mounting methods (door mount, mobile stand, fixed stand) for a light therapy device 300.

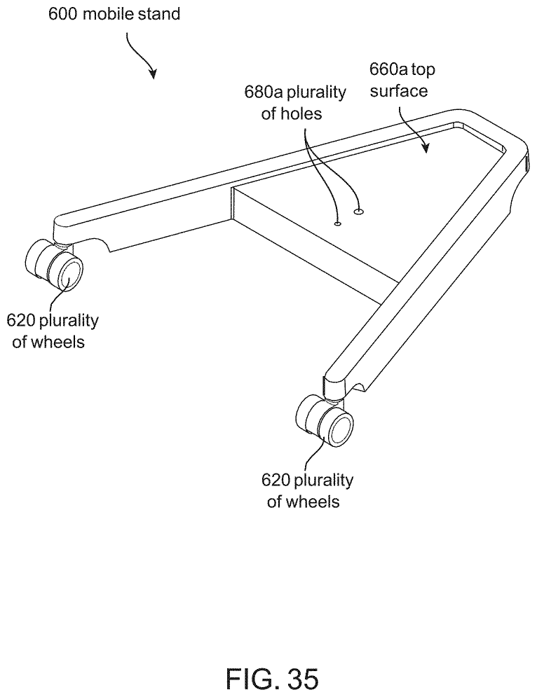

Referring now to FIG. 35, the system 100 may include a mobile stand 600. In some embodiments, the mobile stand 600 comprises a plurality of wheels 620. The plurality of wheels 620 may include two, three, four, or more than four wheels. In some embodiments, the mobile stand 600 includes a single wheel. The mobile stand 600 may include an alternative to a traditional wheel, including but not limited to, a slideable surface (e.g., sled), a continuous track, and/or a pedrail wheel.

FIG. 35 also shows that, in many embodiments, the mobile stand 600 includes a top surface 660a. The top surface 660a may include a plurality of holes 680a. In many embodiments, the plurality of holes 680a is configured to couple the mobile stand 600 to the elongate pole 120 via the bottom base 580. FIG. 34 illustrates that the bottom base 580 may include at least one coupling mechanism 700, and, in some embodiments, the plurality of holes 680a are configured to receive the at least one coupling mechanism 700. The at least one coupling mechanism 700 may include any number of suitable coupling mechanisms, including, but not limited to, fasteners such as a screw(s), a bolt(s), a nut(s), a machine screw(s), and any combination thereof. The plurality of holes 680a may be located anywhere on the top surface 660a. In some embodiments, the bottom base 580 comprises a pad configured to couple between the bottom base 580 and the mobile stand 600. The pad may be configured to act as a buffer; for example, when the bottom base 580 and mobile stand 600 comprise metallic materials, the pad may comprise a plastic material.

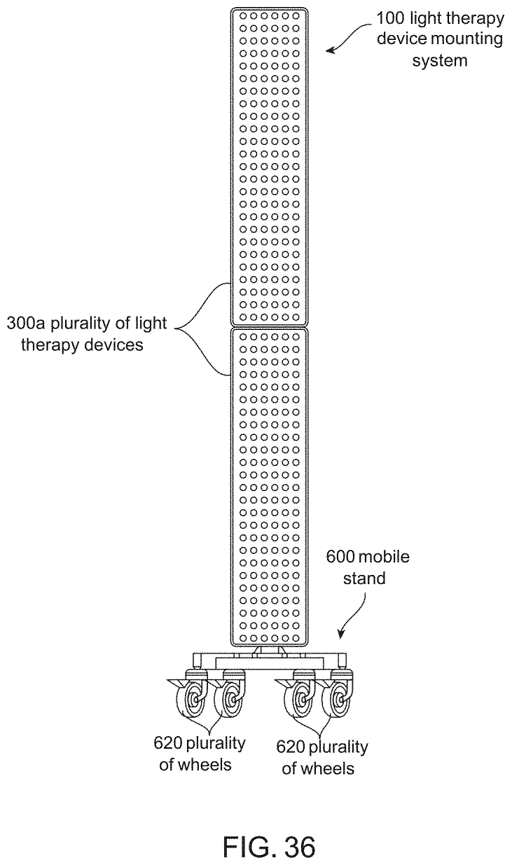

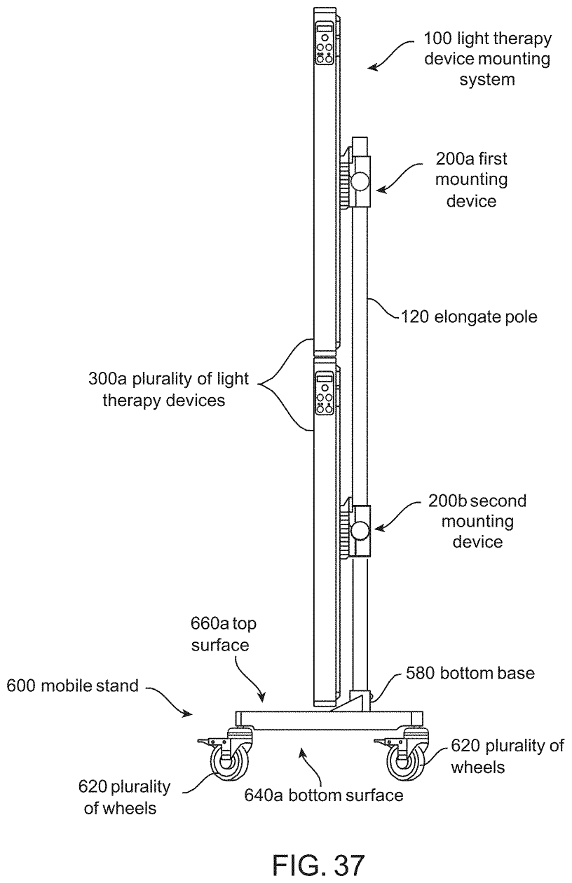

FIG. 36 illustrates a front view of the system 100 with a plurality of light therapy devices 300a coupled to the mobile stand 600. As previously stated, in some embodiments, the plurality of wheels 620 includes four wheels. FIG. 37 shows a side view of the system 100, including a plurality of light therapy devices 300a coupled to a mobile stand 600. As discussed with reference to FIG. 34, in some embodiments, the elongate pole 120 comprises a first mounting device 200a and a second mounting device 200b. FIG. 37 shows the elongate pole 120 with the first and second mounting devices 200a, 200b, where the use of first and second mounting devices 200a, 200b allows for the coupling of a plurality of light therapy devices 300a to the elongate pole 120. In some embodiments, each light therapy device 300 is smaller than the devices 300a shown in FIGS. 36 and 37. As such, the plurality of light therapy devices 300a may comprise more than two devices 300a. For example, three light therapy devices 300a may couple to the elongate pole 120 via three mounting devices 200. In some embodiments, more than three light therapy devices 300a and more than three mounting devices 200 are sized and configured to couple to the elongate pole 120. The elongate pole 120 may define a height greater than what is depicted in the Figures, such that the elongate pole 120 is sized and configured to fit more than two light therapy devices 300a of the size shown in FIGS. 36 and 37.

FIG. 37 also shows the bottom base 580 coupled to the top surface 660a of the mobile stand 600. In some embodiments, the bottom base 580 is larger than the base 580 depicted in FIG. 37, such that the base 580 extends further across the top surface 660a of the stand 600. Stated differently, the bottom base 580 may comprise a larger “footprint” on the top surface 660a such that it takes up a larger portion of the top surface 660a. The bottom base 580 may also define a greater height such that it is configured to receive a greater portion of the elongate pole 120. In some embodiments, the bottom base 580 is smaller than the embodiment shown in FIG. 37. The Figure also shows that, in many embodiments, the plurality of wheels 620 is coupled to a bottom surface 640a of the mobile stand 600. The bottom surface 640a may be located opposite from the top surface 660a.

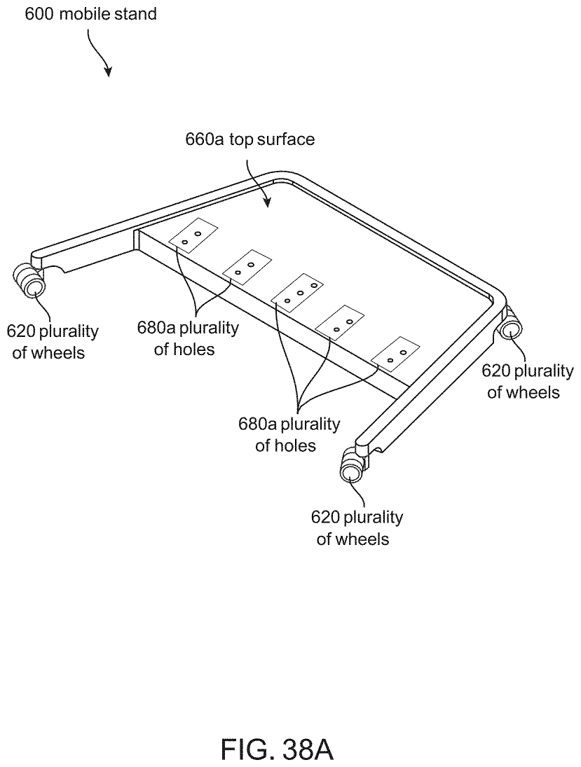

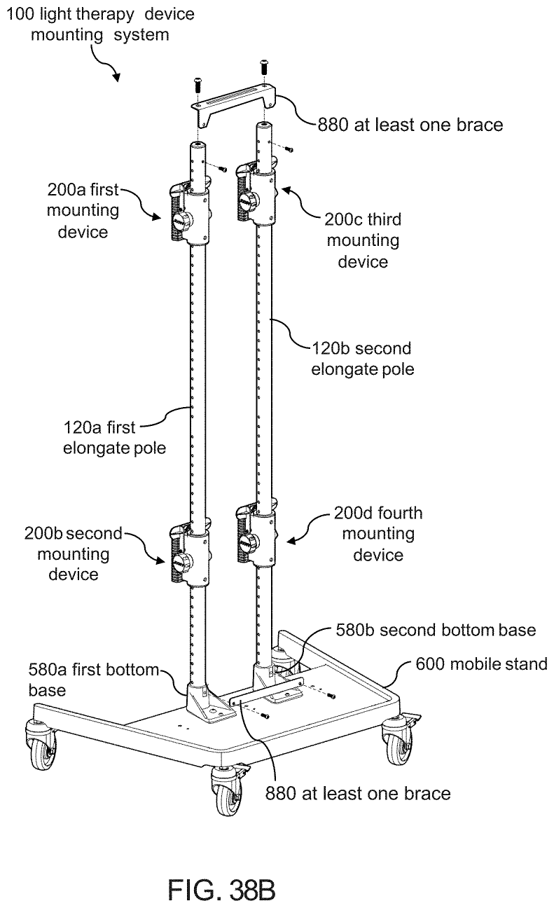

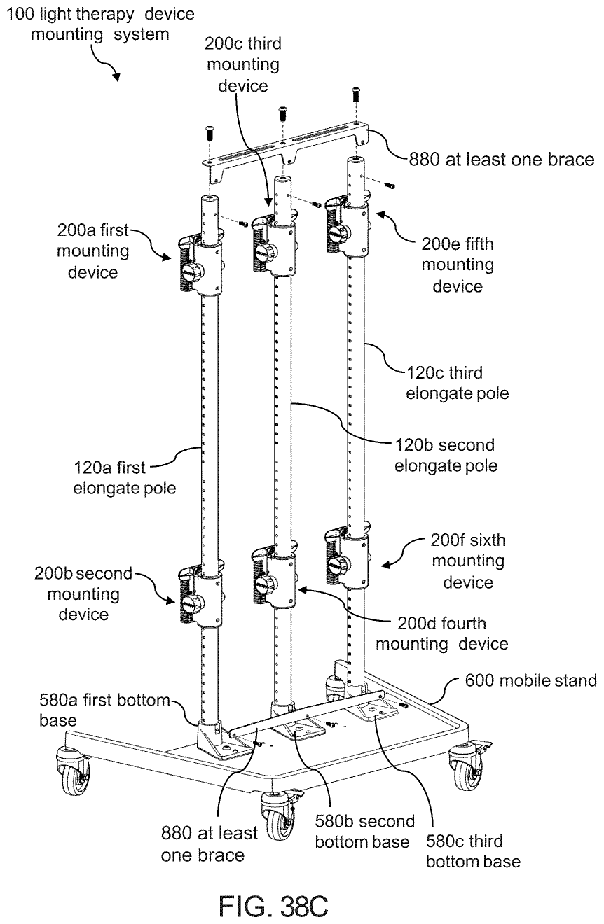

FIG. 38A illustrates another embodiment of the mobile stand 600. As shown, in some embodiments, the mobile stand 600 is larger than the stand 600 shown in FIG. 35. A larger embodiment of the mobile stand 600 may include a more numerous plurality of holes 680a on the top surface 660a. For example, as shown in FIG. 38A, the mobile stand 600 may include five sets in the plurality of holes 680a, as compared to the single set shown on the mobile stand 600 in FIG. 35. In some embodiments, the mobile stand 600 includes a plurality of holes 680a comprising two, three, or four sets of holes. The mobile stand 600 may include more than five sets of holes in the plurality of holes 680a. It should be noted that each “set” of holes in the plurality of holes 680a may include one, two, three, four, or more than four holes. In some embodiments, each “set” of holes is configured to couple to a bottom base 580. As such, the mobile stand 600 shown in FIG. 38A may be sized and configured to couple to up to five bottom bases 580, and therefore, up to five elongate poles 120.

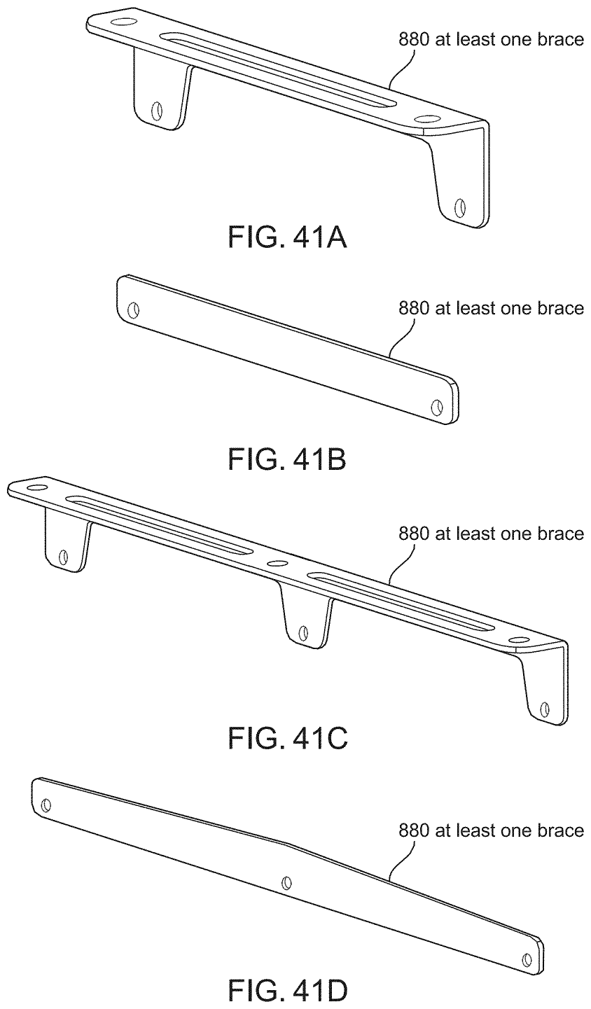

For example, as shown in FIG. 38B, the mobile stand 600 may be configured to couple to a first bottom base 580a and a second bottom base 580b, and therefore to a first elongate pole 120a and a second elongate pole 120b. The system 100 illustrated in FIG. 38B may be configured to include up to four light therapy devices in the plurality of light therapy devices 300a; with one light therapy device 300 coupled to each of the first mounting device 200a, second mounting device 200b, third mounting device 200c, and fourth mounting device 200d. FIG. 38B also shows that, in some embodiments, the system 100 includes at least one brace 880 configured to couple to the first elongate pole 120a and second elongate pole 120b. The at least one brace 880 will be discussed in greater detail with reference to FIGS. 41A-41D.

FIG. 38C is similar to FIG. 38B, but includes an additional third elongate pole 120c to further demonstrate the ability of the mobile stand 600 to support multiple elongate poles 120. As shown, the mobile stand 600 may be configured to couple to a first bottom base 580a, a second bottom base 580b, and a third bottom base 580c, and therefore to a first elongate pole 120a, a second elongate pole 120b, and a third elongate pole 120c. The system 100 illustrated in FIG. 38C may be configured to include up to six light therapy devices in the plurality of light therapy devices 300a; with one light therapy device 300 coupled to each of the first mounting device 200a, second mounting device 200b, third mounting device 200c, fourth mounting device 200d, fifth mounting device 200e, and sixth mounting device 200f FIG. 38C also shows that, in some embodiments, the system 100 includes at least one brace 880 configured to couple to the first elongate pole 120a, second elongate pole 120b, and third elongate pole 120c. As mentioned above, the at least one brace 880 will be discussed later in the disclosure.

Instead of a plurality of holes 680a coupled to at least one coupling mechanism 700, the bottom base 580 may be configured to couple to the top surface 660a of the mobile stand 600 via other coupling methods, including, but not limited to, adhesive, magnet(s), welding, and the like. The bottom base 580 may be fixedly coupled to the mobile stand 600 (e.g., adhesive, welding, etc.). In some embodiments, the bottom base 580 is detachably coupled to the mobile stand 600 (magnets, plurality of holes 680a with at least one coupling mechanism 700, etc.). In some embodiments, the bottom base 580 comprises a pad configured to couple between the bottom base 580 and the mobile stand 600. The pad may be configured to act as a buffer; for example, when the bottom base 580 and mobile stand 600 comprise metallic materials, the pad may comprise a plastic material.

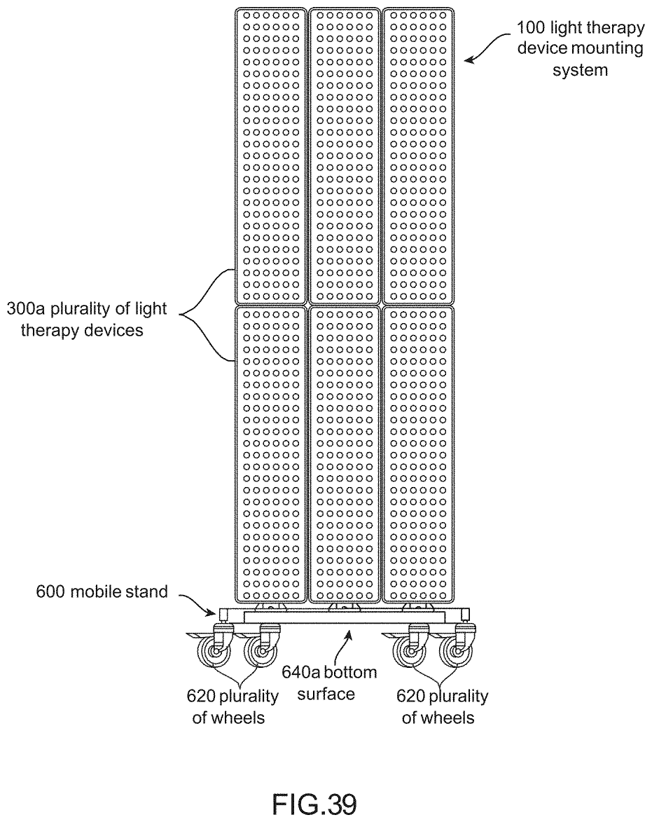

FIG. 39 shows a front view of a plurality of light therapy devices 300a coupled to the mobile stand 600. Similar to the mobile stand 600 shown in FIGS. 35-37, the mobile stand 600 of FIGS. 38-40 and 42 may include a plurality of wheels 620. The plurality of wheels 620 may include two, three, four, or more than four wheels. In some embodiments, the mobile stand 600 includes a single wheel. The mobile stand 600 may include an alternative to a traditional wheel, including but not limited to, a slideable surface (e.g., sled), a continuous track, and/or a pedrail wheel. In many embodiments, the plurality of wheels 620 (or alternative device) is coupled to a bottom surface 640a of the mobile stand 600. The bottom surface 640a may be located opposite the top surface 660a, such that the plurality of wheels 620 may be coupled to a surface of the mobile stand 600 opposite from the surface of the mobile stand 600 coupled to the bottom base(s) 580. FIG. 39 also shows that the plurality of light therapy devices 300a may include more than two light therapy devices 300a. In some embodiments, as shown in FIG. 39, the plurality of light therapy devices 300a comprises six light therapy devices 300a. The plurality of light therapy devices 300a may comprise three, four, or five light therapy devices 300a. In some embodiments, the plurality of light therapy devices 300a comprises more than six light therapy devices 300. A single light therapy device 300 may be coupled to the mobile stand 600.

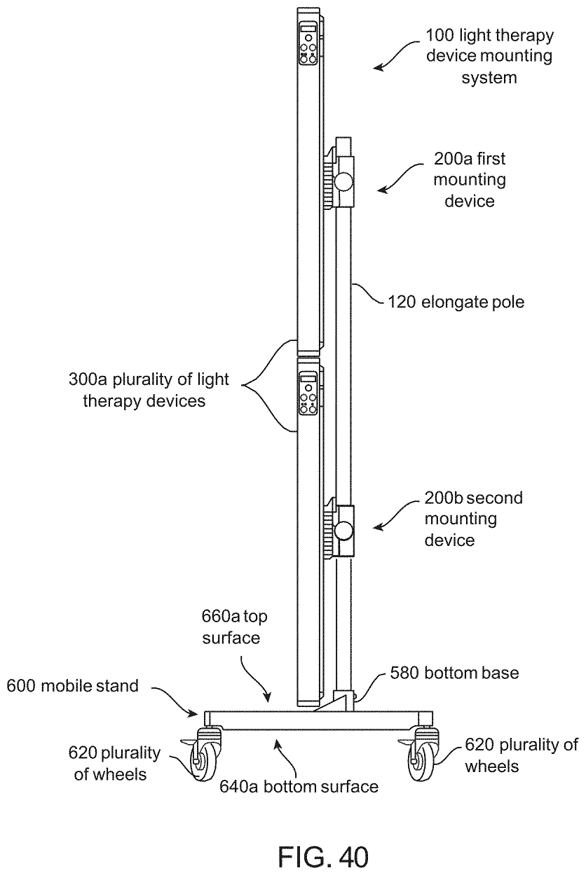

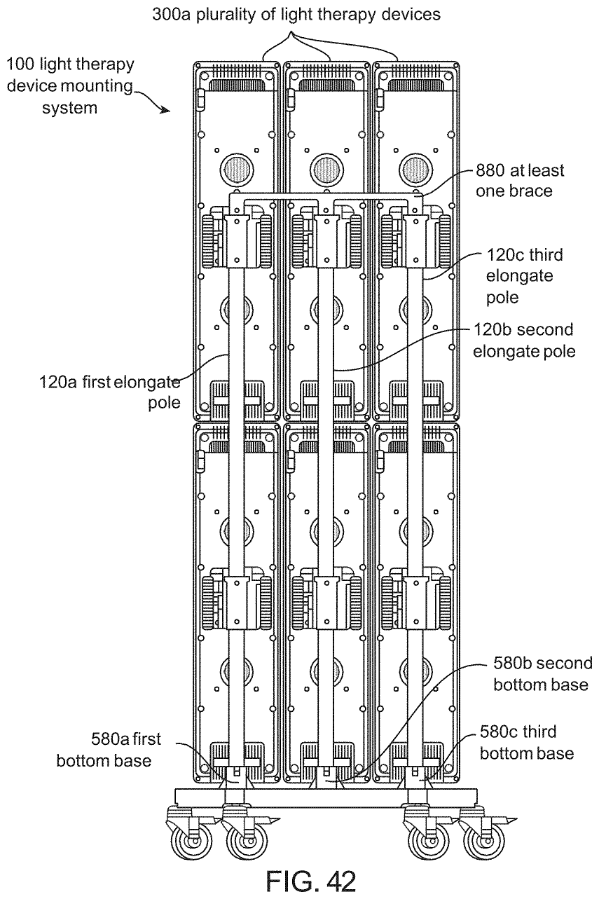

Similar to FIG. 37, FIG. 40 illustrates a side view of the system 100, including a plurality of light therapy devices 300a coupled to a mobile stand 600. Though shown with only two light therapy devices 300a in the plurality of light therapy devices 300a, it should be noted that FIG. 40 may be considered to show a side view of the same embodiment of the system 100 depicted in FIG. 39. As will be discussed further with reference to FIG. 42, the system 100 shown in FIGS. 39 and 40 may include three elongate poles 120, where each pole 120 comprises a first and second mounting device 200a, 200b. Each mounting device 200a, 200b may be configured to couple to a light therapy device 300. As such, the use of three elongate poles 120, where each pole 120 is coupled to two light therapy devices 300a, facilitates the embodiment of the system 100 shown in FIG. 39 with six light therapy devices 300a.

FIG. 40 also shows the bottom base 580 coupled to the top surface 660a of the mobile stand 600. In some embodiments, the bottom base 580 is larger than the base 580 depicted in FIG. 40, such that the base 580 extends further across the top surface 660a of the stand 600. Stated differently, the bottom base 580 may comprise a larger “footprint” on the top surface 660a such that it takes up a larger portion of the top surface 660a. The bottom base 580 may also define a greater height such that it is configured to receive a greater portion of the elongate pole 120. In some embodiments, the bottom base 580 is smaller than the embodiment shown in FIG. 40.

FIGS. 41A, 41B, 41C, and 41D illustrate embodiments of at least one brace 880. In many embodiments, the at least one brace 880 is configured to provide stability to the system 100 when the system 100 includes multiple elongate poles 120 coupled to a mobile stand 600. The at least one brace 880 may be sized and configured to couple to a top portion of at least one elongate pole 120. For example, the at least one brace 880 shown in FIGS. 41A and 41B may be configured to couple to two elongate poles 120, and the at least one brace 880 shown in FIGS. 41C and 41D may be configured to couple to three elongate poles 120. In some embodiments, the at least one brace 880 shown in FIGS. 41A and 41C is configured to couple to the top portions of the elongate poles 120, and the at least one brace 880 shown in FIGS. 41B and 41D is configured to couple to lower portions on the elongate poles 120. The at least one brace 880 of FIGS. 41B and 41D may be configured to couple to the bottom base 580 of each elongate pole 120. The at least one brace 880 may be configured to couple to each elongate pole 120 at the top surface and at least one other point on the elongate pole 120. For example, the at least one brace 880 shown in FIGS. 41A and 41C may be configured to couple to each elongate pole 120 at the top surface and a back or front surface located adjacent the top surface, such that the at least one brace 880 “folds over” the top surface and extends slightly down the length of each elongate pole 120. In some embodiments, the at least one brace 880 shown in FIGS. 41A and 41C is configured to couple to a top surface and a back surface, and the at least one brace 880 shown in FIGS. 41B and 41D is configured to couple to the back surface, of each elongate pole 120. The back surface may be located opposite the front surface, where the front surface comprises the surface of the elongate pole 120 located adjacent the mounting device bracket 400 (e.g., the “front surface” is located closer to the light therapy device 300 than the “back surface”).

The at least one brace 880 may be configured to detachably couple to each elongate pole 120 via any of the coupling mechanisms mentioned in this disclosure. The at least one brace 880 may be configured to fixedly couple to each elongate pole 120 via any of the coupling mechanisms mentioned in this disclosure. For example, the holes shown on the at least one brace 880 (in FIGS. 41A-41D) may be configured to receive at least one coupling mechanism 700 in a manner similar to the plurality of holes 680a located on the mobile stand 600. The elongate pole 120 may include holes configured to receive at least one coupling mechanism 700. These holes may be separate from the plurality of holes 140. In some embodiments, at least one hole of the plurality of holes 140 is configured to receive at least one coupling mechanism 700 in order to couple the elongate pole 120 to at least one brace 880.

FIG. 42 shows a back view of the system 100 depicted in FIGS. 39 and 40. In some embodiments, the system 100 includes a plurality of light therapy devices 300a comprising six light therapy devices 300a, a first elongate pole 120a, a second elongate pole 120b, a third elongate pole 120c, a first bottom base 580a, a second bottom base 580b, and a third bottom base 580c. The first bottom base 580a may be configured to receive the first elongate pole 120a, the second bottom base 580b may be configured to receive the second elongate pole 120b, and the third bottom base 580c may be configured to receive the third elongate pole 120c. As previously discussed, each elongate pole 120 may be configured to couple to two light therapy devices 300a via a first and second mounting device 200a, 200b. FIG. 42 also includes the at least one brace 880, which is shown extending across the top portions of the first, second, and third elongate poles 120a, 120b, and 120c. The at least one brace 880 may be configured to provide stability to the system 100 by coupling the elongate poles 120a, 120b, and 120c. The stability may be important to reduce movement of each elongate pole 120 during movement of the system 100, such as when the mobile stand 600 is rolled from one area to another, or in case of a sudden movement, like an earthquake or a person bumping into the system 100. The stability from the at least one brace 880, combined with the stability from each bottom base 580 coupled to the mobile stand 600, may provide a substantial amount of support and stability to the system 100 and protect the plurality of light therapy devices 300a from damage.

As stated with reference to FIGS. 38A-38C, the mobile stand 600 may be configured to support up to five elongate poles 120. FIG. 42 demonstrates that the number of elongate poles 120 in the system 100 may be dependent on the size of each light therapy device 300 of the plurality of light therapy devices 300a. For example, FIG. 42 includes three elongate poles 120a, 120b, and 120c and shows, along with FIG. 39, that the plurality of light therapy devices 300a does not include much space between each light therapy device 300. As such, the embodiment shown in FIGS. 35 and 38 may not be suitable for more than three elongate poles 120a, 120b, and 120c. In some embodiments, each light therapy device 300 comprises a narrower light therapy device 300 than the devices 300a shown in the Figures. In such an embodiment, there may be enough room to accommodate four or five elongate poles 120 on the mobile stand 600. In contrast, each light therapy device 300 may comprise a wider light therapy device 300 than the devices 300a shown in the Figures. In such an embodiment, the stand 600 shown in FIGS. 38-40 and 42 may only accommodate one or two elongate poles 120. Depending on the size of each light therapy device 300, each device 300 may be configured to couple to a first elongate pole 120a and a second elongate pole 120b in order to provide sufficient support for each light therapy device 300.

### Fixed Stand System Embodiments

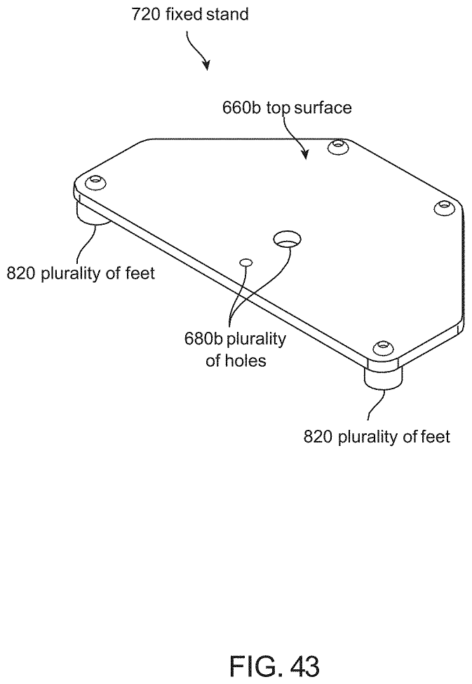

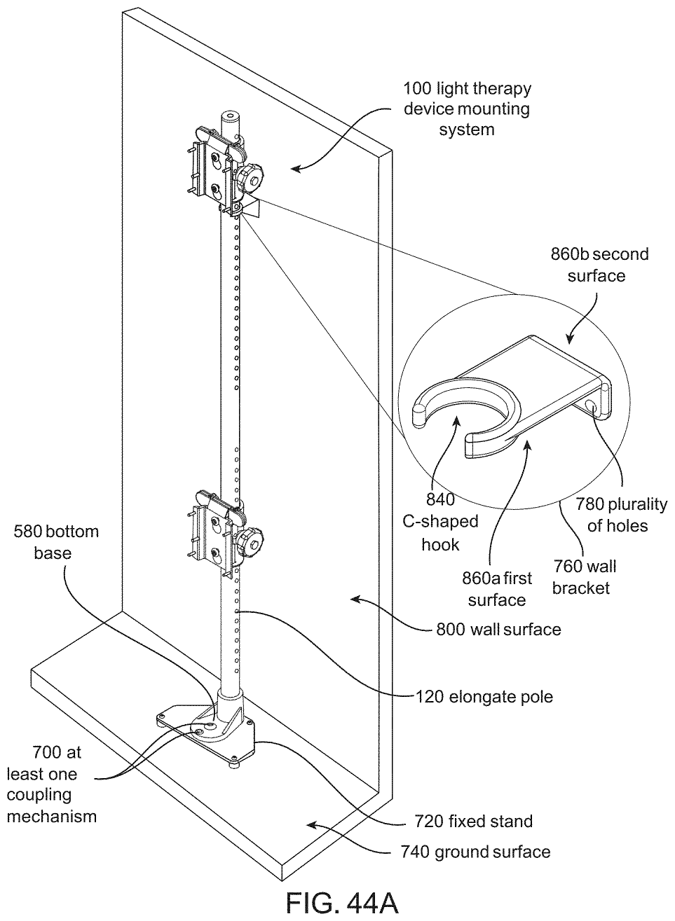

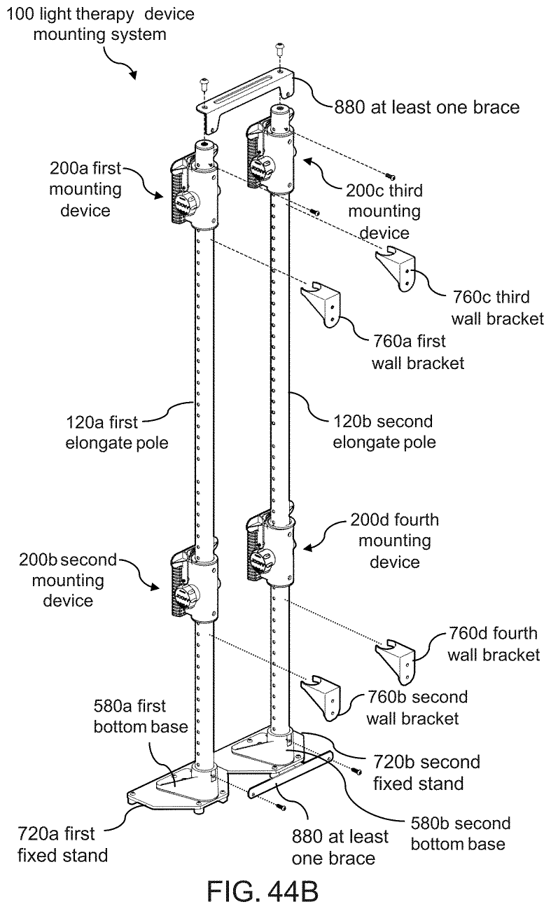

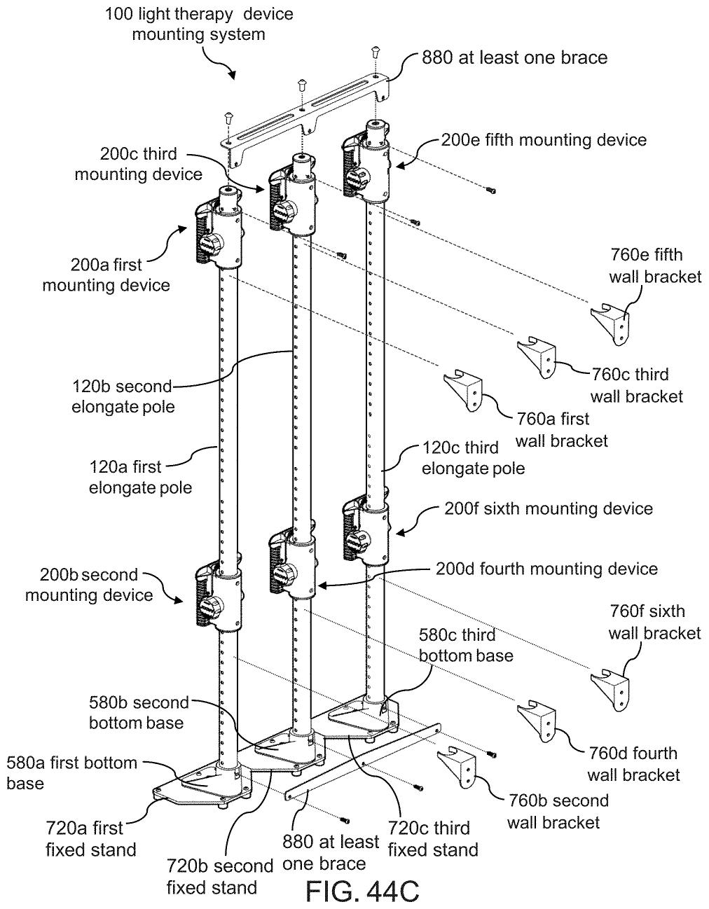

FIG. 43 shows an embodiment of a fixed stand 720, including a top surface 660b, a plurality of holes 680b, and a plurality of feet 820. The plurality of feet 820 may include two, three, four, or more than four feet configured to contact a ground surface 740 (as shown in FIGS. 44A-44C). In some embodiments, the fixed stand 720 includes a single foot configured to contact the ground surface 740. The plurality of feet 820 may be located on a bottom surface 640b of the fixed stand 720 located opposite the top surface 660b. Similar to the mobile stand 600, in many embodiments, the plurality of holes 680b is configured to couple the fixed stand 720 to the elongate pole 120 via the bottom base 580.

FIG. 44A illustrates that the bottom base 580 may include at least one coupling mechanism 700, and, in some embodiments, the plurality of holes 680b is configured to receive the at least one coupling mechanism 700. The at least one coupling mechanism 700 may include any number of suitable coupling mechanisms, including, but not limited to, fasteners such as a screw(s), a bolt(s), a nut(s), a machine screw(s), and any combination thereof. The plurality of holes 680b may be located anywhere on the top surface 660b. In some embodiments, the bottom base 580 comprises a pad configured to couple between the bottom base 580 and the fixed stand 720. The pad may be configured to act as a buffer; for example, when the bottom base 580 and fixed stand 720 comprise metallic materials, the pad may comprise a plastic material.

Instead of a plurality of holes 680b coupled to at least one coupling mechanism 700, the bottom base 580 may be configured to couple to the top surface 660b of the fixed stand 720 via other coupling methods, including, but not limited to, adhesive, magnet(s), welding, and the like. The bottom base 580 may be fixedly coupled to the fixed stand 720 (e.g., adhesive, welding, etc.). In some embodiments, the bottom base 580 is detachably coupled to the fixed stand 720 (magnets, plurality of holes 680b with at least one coupling mechanism 700, etc.).

Though illustrated as an irregular hexagon, it should be noted that the fixed stand 720 may define any shape suitable to support at least one bottom base 580 coupled to at least one elongate pole 120 and at least one light therapy device 300. For example, the fixed stand 720 may define a round, ovoid, triangular, rectangular, pentagonal, hexagonal, heptagonal, etc. shape. The fixed stand 720 may also be sized to include multiple sets of holes in the plurality of holes 680b, similar to the mobile stand 600 shown in FIGS. 38A-38C, such that the fixed stand 720 may accommodate multiple elongate poles 120 via coupling to multiple bottom bases 580. Each foot of the plurality of feet 820 may define any suitable shape.

In some embodiments, as illustrated in FIG. 44A, the system 100 includes a wall bracket 760 coupled to the elongate pole 120 when the elongate pole 120 is coupled to the fixed stand 720. The wall bracket 760 may include a C-shaped hook 840 located on a first surface 860a of the wall bracket 760, as well as a plurality of holes 780 located on a second surface 860b of the wall bracket 760. The first surface 860a may be located opposite the second surface 860b. In many embodiments, the C-shaped hook 840 is configured to flex in order to snapably receive the elongate pole 120 to thereby couple the elongate pole 120 to the wall bracket 760, and thereby, to couple the elongate pole 120 to the wall surface 800. The C-shaped hook 840 may be configured to couple to the elongate pole 120 in another manner, such as slideably receiving the elongate pole 120. The C-shaped hook 840 may comprise another suitable shape, such as “D” or “0.” In some embodiments, the C-shaped hook 840 comprises a hinged closure, similar to that commonly found on a carabiner. The C-shaped hook 840 may comprise a shape configured to substantially surround the elongate pole 120 when the wall bracket 760 is coupled to the elongate pole 120. The C-shaped hook 840 may be configured to couple to any suitable portion of the elongate pole 120, including a first end 160, a second end 180, and/or a middle portion.

In many embodiments, the second surface 860b is configured to contact a wall surface 800, and the plurality of holes 780 is configured to receive a coupling mechanism in order to couple the wall bracket 760 to the wall surface 800. The coupling mechanism may be the same as the at least one coupling mechanism 700. In some embodiments, the coupling mechanism configured to couple the wall bracket 760 to the wall surface 800 comprises at least one drywall screw or similar mechanism. The plurality of holes 780 may comprise two, three, four, or more than four holes. The holes may be configured in a side-by-side or top-to-bottom pattern. In some embodiments, the plurality of holes 780 comprises a single hole.

FIGS. 44B and 44C illustrate back perspective views of the system 100 comprising multiple elongate poles 120, where each elongate pole 120 is coupled to a fixed stand 720. FIG. 44B includes a first elongate pole 120a and a second elongate pole 120b, while FIG. 44C includes a first elongate pole 120a, a second elongate pole 120b, and a third elongate pole 120c. In many embodiments, the first elongate pole 120a is configured to be coupled to a first bottom base 580a and a first fixed stand 720a. The first elongate pole 120a may also comprise two mounting devices 200, a first mounting device 200a and a second mounting device 200b. Similarly, the second elongate pole 120b may be coupled to a second bottom base 580b and a second fixed stand 720b, as well as a third mounting device 200c and a fourth mounting device 200d. As shown in FIG. 44C, the third elongate pole 120c may be coupled to a third bottom base 580c and a third fixed stand 720c, as well as a fifth mounting device 200e and a sixth mounting device 200f. In many embodiments, each mounting device 200a, 200b, 200c, 200d, 200e, 200f is configured to couple to a light therapy device 300. Accordingly, the system 100 of FIG. 44B may be configured to support up to four light therapy devices 300, and the system 100 of FIG. 44C may be configured to support up to six light therapy devices 300.

FIGS. 44B and 44C also include a plurality of wall brackets 760, similar to the wall bracket 760 shown in FIG. 44A. In some embodiments, as illustrated in FIGS. 44B and 44C, each elongate pole 120 may be configured to couple to two wall brackets 760, as compared to the single wall bracket 760 shown in FIG. 44A. Each elongate pole 120 may be configured to couple to more than two wall brackets 760. The inclusion of more than one wall bracket 760 per elongate pole 120 may increase stability of the system 100 by providing an additional anchor point(s) to couple the system 100 to a wall surface 800. FIGS. 44B and 44C also include at least one brace 880 configured to couple together multiple elongate poles 120, as discussed with reference to FIGS. 41A-41D.

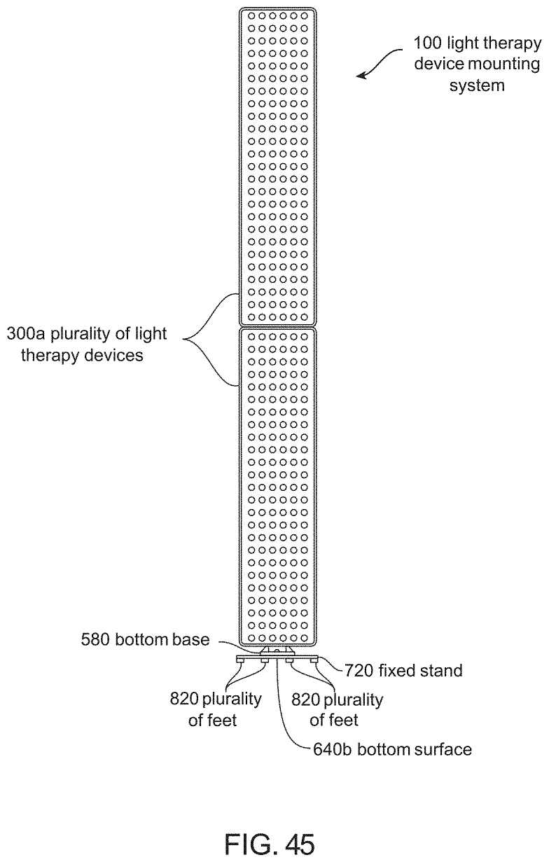

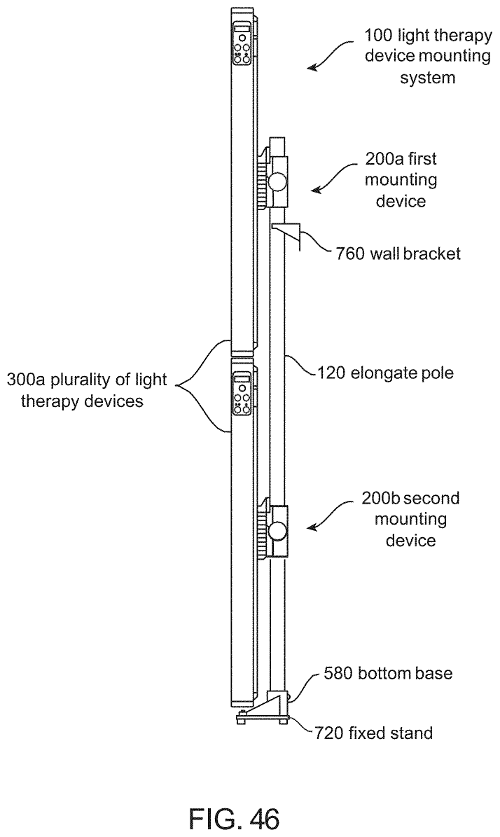

FIG. 45 shows a front view of the system 100, including a plurality of light therapy devices 300a coupled to the fixed stand 720 via the bottom base 580. As previously stated, in some embodiments, the plurality of feet 820 includes four feet. The plurality of feet 820 may be coupled to a bottom surface 640b of the fixed stand 720. FIG. 46 shows a side view of the system 100, including a plurality of light therapy devices 300a coupled to a fixed stand 720. As discussed with reference to FIG. 34, in some embodiments, the elongate pole 120 comprises a first mounting device 200a and a second mounting device 200b. FIG. 46 shows the elongate pole 120 with the first and second mounting devices 200a, 200b, where the use of first and second mounting devices 200a, 200b allows for the coupling of a plurality of light therapy devices 300a to the elongate pole 120.

FIG. 46 also shows that, in some embodiments, the wall bracket 760 is located between the first mounting device 200a and the second mounting device 200b. The wall bracket 760 may be located above the first mounting device 200a. The wall bracket 760 may be located below the second mounting device 200b. In some embodiments, the wall bracket 760 is located closer to the first mounting device 200a than the second mounting device 200b. The wall bracket 760 may be located closer to the second mounting device 200b than the first mounting device 200a. The wall bracket 760 may be located substantially equidistant from the first mounting device 200a and the second mounting device 200b. Some embodiments of the system 100 include a plurality of wall brackets 760 coupled to the elongate pole 120. The system 100 may include a plurality of elongate poles 120, with at least one wall bracket 760 coupled to each elongate pole 120. FIGS. 44-46 depict the bottom base 580 coupled to the top surface 660b of the fixed stand 720. In some embodiments, the bottom base 580 is larger than the base 580 depicted in FIGS. 44, 45, and/or 46, such that the base 580 extends further across the top surface 660b of the stand 720. Stated differently, the bottom base 580 may comprise a larger “footprint” on the top surface 660b such that it takes up a larger portion of the top surface 660b. The bottom base 580 may also define a greater height such that it is configured to receive a greater portion of the elongate pole 120. In some embodiments, the bottom base 580 is smaller than the embodiments shown in FIGS. 44, 45, and/or 46.

As previously mentioned, the fixed stand 720 may be sized to accommodate multiple bottom bases 580 such that multiple elongate poles 120 may be supported by the fixed stand 720. In some embodiments, when the fixed stand 720 is coupled to a plurality of elongate poles 120, the system 100 includes at least one brace 880, similar to those illustrated in FIGS. 41A-41D. The at least one brace 880 may also be used when the system 100 comprises multiple fixed stands 72 located adjacent one another. For example, in some embodiments, the system 100 includes three fixed stands 72 located adjacent one another, each fixed stand 720 comprising an elongate pole 120 coupled to at least one light therapy device 300. The system 100 may also include at least one brace 880, such as the brace illustrated in FIG. 41B, configured to couple to each elongate pole 120 to provide stability to the system 100.

### Floor Stand System Embodiments

Referring now to FIG. 47, a light therapy device floor stand 900 may include a floor plate 920 comprising a first curved side arm 940a and a second curved side arm 940b. In many embodiments, the first curved side arm 940a and second curved side arm 940b are coupled to a top surface 960 of the floor plate 920. At least one of the first curved side arm 940a and the second curved side arm 940b may be coupled to the top surface 960a via any of the coupling mechanisms mentioned in this disclosure. At least one of the first curved side arm 940a and the second curved side arm 940b may be fixedly coupled to the floor plate 920. In some embodiments, at least one of the first curved side arm 940a and the second curved side arm 940b is detachably coupled to the floor plate 920. The first curved side arm 940a may be located at a first side 980a of the floor plate 920, and the second curved side arm 940b may be located at a second side 980b of the floor plate 920. As shown in FIG. 47, the first curved side arm 940a and the second curved side arm 940b may be configured to open facing toward one another. The first and second curved side arms 940a, 940b may define shapes similar to open and closed square brackets, respectively.

In some embodiments, as shown in FIG. 47, the top surface 960 comprises an indented portion (e.g., a channel, groove, trench, rut, or the like), and at least one of the first curved side arm 940a and the second curved side arm 940b is configured to couple to the indented portion. By coupling to the indented portion, at least one of the first curved side arm 940a and second curved side arm 940b may be configured to sit at least partially in the floor plate 920 and at least partially below the top surface 960. The top surface 960 may comprise a substantially continuous surface without an indented portion, such that at least one of the first curved side arm 940a and the second curved side arm 940b is configured to sit on top of the floor plate 920. Disregarding the indented portion, the floor plate 920 may comprise a substantially flat and level top surface 960. In some embodiments, as shown in FIG. 47 and disregarding the indented portion, the floor plate 920 comprises a bowed top surface 960 such that the outer portions of the top surface 960 located furthest from the first and second curved side arms 940a, 940b define a lesser height than a middle portion of the top surface 960. The outer portion of the top surface 960 located furthest from the first and second curved side arms 940a, 940b may define a greater height than the middle portion of the top surface 960.

In some embodiments, the floor plate 920 comprises a substantially rectangular base. It should be noted that the “base” may be considered a bottom surface of the floor plate 920 located opposite the top surface 960, wherein the base is configured to contact a floor surface. The base may define any suitable shape, including but not limited to round, ovoid, triangular, pentagonal, hexagonal, etc. In some embodiments, the floor plate 920 comprises at least one wheel and/or at least one foot coupled to the base of the floor plate 920. It should be noted that though called a light therapy device “floor” stand 900, the stand 900 may be configured to restably couple to a table, counter, desk, chair, stool, or any other indoor or outdoor surface. The floor stand 900 should not be considered to be limited to use on a floor.

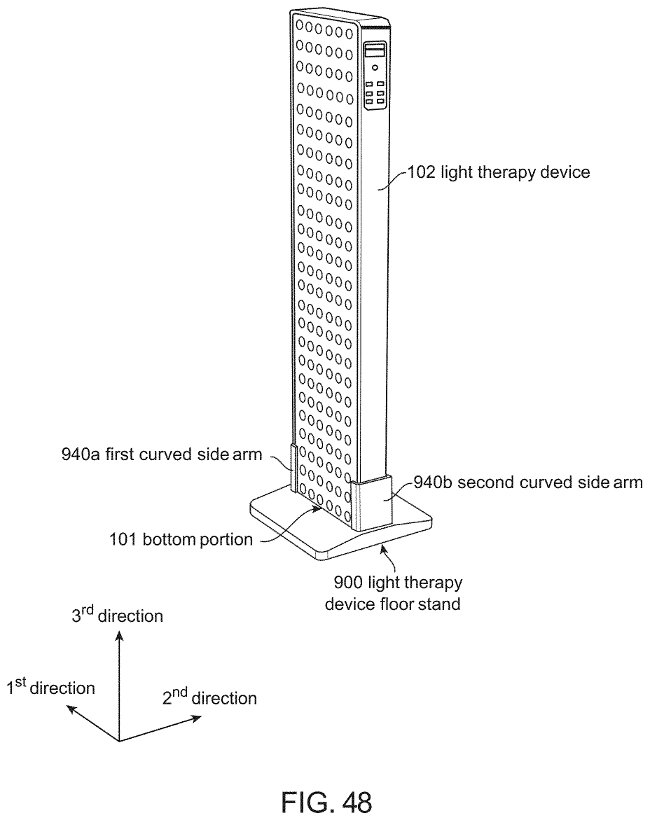

FIG. 48 illustrates the floor stand 900 coupled to a light therapy device 102. The light therapy device 102 may define any number of sizes and configurations. In some embodiments, the light therapy device 102 is substantially identical to the light therapy device 300 mentioned throughout the disclosure. Each of the light therapy device 300 and the light therapy device 102 may comprise a light therapy device produced by Joovv, Inc. of Delaware, USA. For example, the light therapy device 300, 102 may comprise any of the devices disclosed in the following U.S. Nonprovisional Patent Applications and/or issued U.S. Patents: application Ser. No. 15/616,028; application Ser. No. 16/167,385; U.S. Pat. Nos. 10,478,635; 10,639,495; application Ser. No. 16/598,033; and application Ser. No. 16/904,243.

The floor stand 900 may be configured to couple to the light therapy device 102 via the first and second curved side arms 940a, 940b. In many embodiments, the first and second curved side arms 940a, 940b are configured to slideably receive a bottom portion 101 of the light therapy device 102. As previously mentioned, the floor stand 900 may include an indented portion on the top surface 960. In some embodiments, the indented portion is configured to receive at least a portion of the bottom portion 101. This may add stability to the light therapy device 102 by coupling the device 102 to the floor stand 900 via both the indentation in the floor plate 920 and the first and second curved side arms 940a, 940b.

In many embodiments, as shown by the directional indicators in FIG. 48, the floor plate 920 may extend along a first direction and a second direction perpendicular to the first direction, and the first and second curved side arms 940a, 940b may extend along a third direction perpendicular to the first direction and second direction. The light therapy device 102 may also extend along the third direction. In some embodiments, at least one of the first curved side arm 940a and the second curved side arm 940b defines a greater height than depicted in FIG. 48. As such, at least one of the first curved side arm 940a and the second curved side arm 940b may be configured to extend further up (i.e., along the third direction) the light therapy device 102. At least one of the first and second curved side arms 940a, 940b may extend along the third direction to a lesser extent than shown in FIG. 48. The first and second curved side arms 940a, 940b may be configured such that a width of the curved side arms 940a, 940b is just slightly larger than a width of the light therapy device 102. As such, the light therapy device 102 may be configured to fit snugly in the first and second curved side arms 940a, 940b with little and/or no space to move, wobble, rock, or the like.

In some embodiments, the light therapy device 102 comprises a plurality of light therapy devices 102 configured to couple to the floor stand 900. For example, the light therapy device 102 may be coupled to another light therapy device 102 in a top-to-bottom stacked formation, wherein the bottom device 102 is coupled to the floor stand 900 via the curved side arms 940a, 940b and the top device 102 is coupled to the bottom device 102 via attachment posts or a similar method. In some embodiments, a light therapy device 102 is coupled adjacent another light therapy device 102 in a side-by-side formation. In such an embodiment, the first curved side arm 940a may be configured to slideably receive an outer portion of one light therapy device 102, and the second curved side arm 940b may be configured to receive an outer portion of the other light therapy device 102. In some embodiments, the floor stand 900 comprises multiple sets of curved side arms 940a, 940b in order to facilitate coupling to a plurality of light therapy devices 102.

### Table Stand System Embodiments

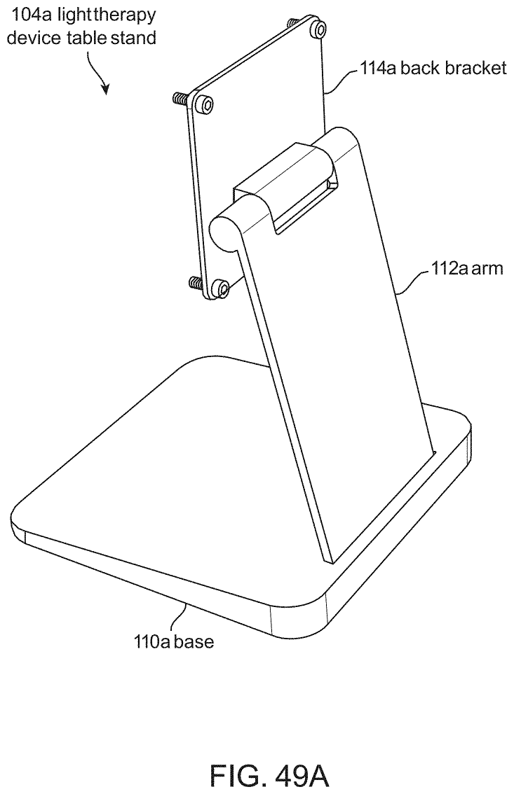

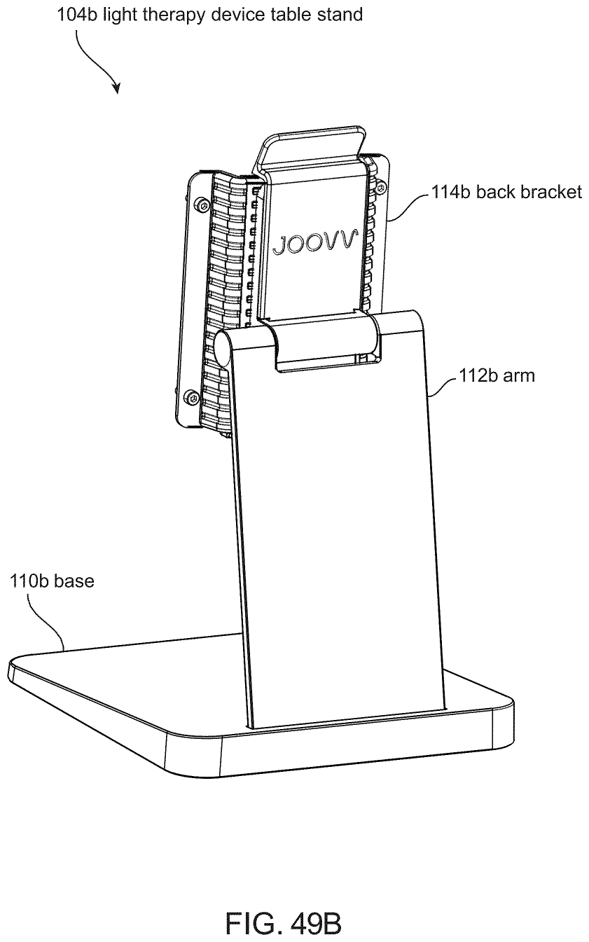

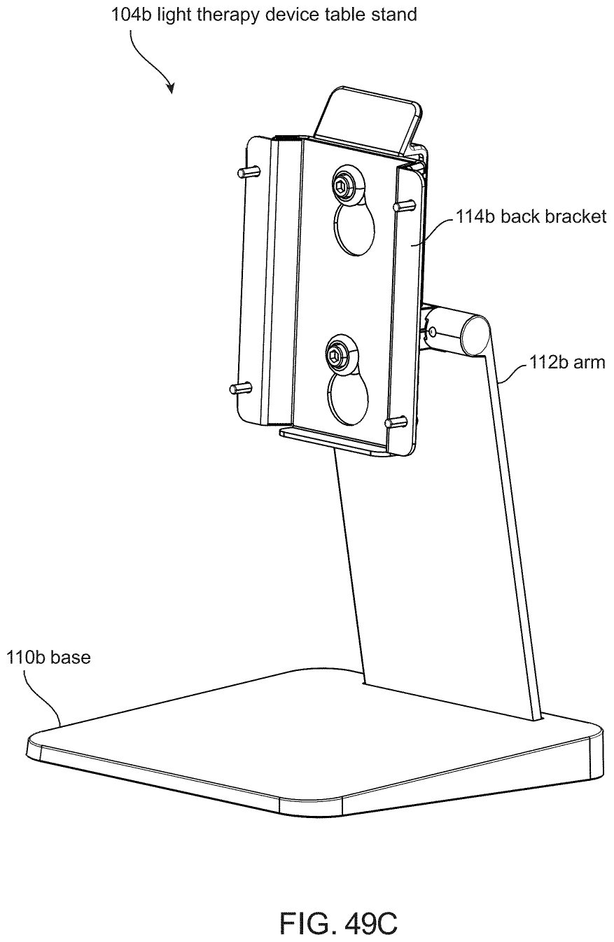

FIGS. 49A and 49B illustrate back perspective views of embodiments of a light therapy device table stand 104a and a light therapy device table stand 104b, respectively. FIG. 49C shows a front perspective view of the light therapy device table stand 104b illustrated in FIG. 49B. It should be noted that though called a “table” stand 104a, 104b, the stand 104a, 104b may be configured to restably couple to a variety of surfaces including, but not limited to, a ground and/or floor surface, a desk, a chair, a stool, a counter, a bed, and any other indoor and/or outdoor surface. In many embodiments, the table stand 104a includes a base 110a, an arm 112a, and a back bracket 114a (as shown in FIG. 49A), and the table stand 104b includes a base 110b, an arm 112b, and back bracket 114b (as shown in FIG. 49B). The base 110 may be configured to contact the table and/or other surfaces.

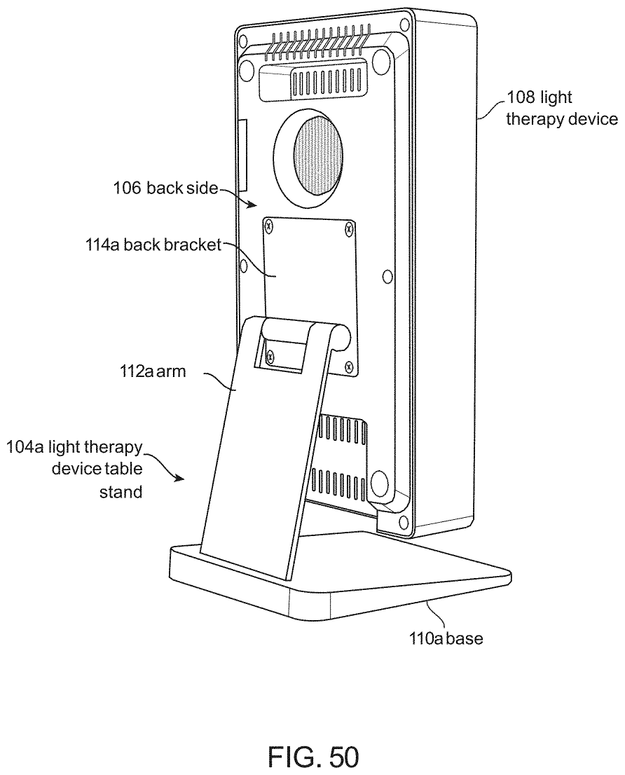

FIG. 50 shows that, in some embodiments, the table stand 104a of FIG. 49A may be configured to couple to a light therapy device 108 via the back bracket 114a. In some embodiments, the back bracket 114a is configured to couple to a back side 106 of the light therapy device 108. The back bracket 114a may detachably couple to the light therapy device 108 via any of the coupling mechanisms disclosed herein. In some embodiments, the back bracket 114a is configured to fixedly couple to the light therapy device 108 via any of the coupling mechanisms disclosed herein. The back bracket 114a may comprise a single bracket, as shown in FIG. 50. In some embodiments, the back bracket 114a comprises a plurality of brackets.

Though not shown in the Figures, the table stand 104b may also be configured to couple to a light therapy device, such as the light therapy device 108, via the back bracket 114b. In many embodiments, the back bracket 114b is configured to couple to the light therapy device 108 in a manner substantially similar to coupling the mounting device 200 to the light therapy device 300 as described earlier in this disclosure, particularly with reference to FIGS. 31-33. In some embodiments, the back bracket 114b is rotatably coupled to the arm 112b.

As demonstrated in FIGS. 49A and 50, the arm 112a may be configured to rotatably couple the back bracket 114a to the base 110a. In many embodiments, the back bracket 114a is adjustable such that the light therapy device 108 is configured to tilt at a wide range of angles. FIG. 50 shows the light therapy device 108 situated substantially straight, which may be suitable for delivering light therapy directly in front of the device 108. In some embodiments, the light therapy device 108 is configured to tilt upwards in order to provide light therapy to an area at an angle above the device 108. For example, a user may stand in front of the device 108 when the stand 104a is restably coupled to a table, and the user may adjust the back bracket 114a such that the device 108 is angled upward in order to emit light toward the upper torso and/or head region of the user. Alternatively, the user may adjust the back bracket 114a such that the device 108 is angled downward in order to emit light towards the lower torso and/or legs of the user. The arm 112a may comprise a single-arm, as shown in FIGS. 49A and 50. In some embodiments, the arm 112a comprises a plurality of arms.

Similarly, with regard to the stand 104b of FIGS. 49B and 49C, the arm 112b may be configured to couple the back bracket 114b to the base 110b. In many embodiments, due to the rotatable coupling, the back bracket 114b is adjustable such that the light therapy device 108 is configured to tilt at a wide range of angles. For example, a user may stand in front of the device 108 when the stand 104b is restably coupled to a table, and the user may adjust the back bracket 114b on the arm 112b such that the device 108 is angled upward in order to emit light toward the upper torso and/or head region of the user. Alternatively, the user may adjust the back bracket 114b on the arm 112b such that the device 108 is angled downward in order to emit light towards the lower torso and/or legs of the user. The arm 112b may comprise a single-arm, as shown in FIGS. 49B and 49C. In some embodiments, the arm 112b comprises a plurality of arms.

The table stand 104a, 104b may be configured to couple to a plurality of light therapy devices 108. In some embodiments, the table stand 104a, 104b comprises a plurality of back brackets 114a, 114b and a plurality of arms 112a, 112b in order to couple to a plurality of light therapy devices 108. The light therapy devices 108 may be configured to couple to one another independent of the table stand 104a, 104b, such that the table stand 104a, 104b is configured to couple to multiple light therapy devices 108 when the table stand 104a, 104b comprises a single arm 112a, 112b and a single back bracket 114a, 114b. For example, the light therapy device 108 may be coupled to another light therapy device 108 in a top-to-bottom stacked configuration such that one light therapy device 108 is coupled on top of another light therapy device 108. In such an embodiment, the back bracket 114a, 114b may be configured to couple to either the top or bottom light therapy device 108.

Similar to the light therapy device 300 and the light therapy device 102, the light therapy device 108 may comprise a light therapy device produced by Joovv, Inc. of Delaware, USA. For example, the light therapy device 300, 102, or 108 may comprise any of the devices disclosed in the following U.S. Nonprovisional Patent Applications and/or issued U.S. Patents: application Ser. No. 15/616,028; aapplication Ser. No. 16/167,385; U.S. Pat. No. 10,478,635; 10,639,495; application Ser. No. 16/598,033; and application Ser. No. 16/904,243.

### Mounting Kit Embodiments

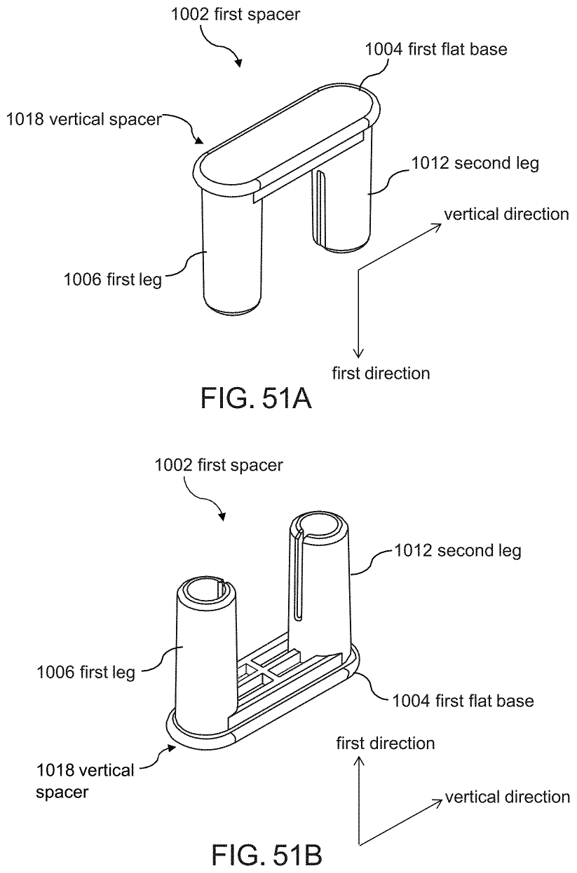

It should be noted that the embodiments shown in, and discussed with reference to, FIGS. 51A-63 may include components previously discussed in this disclosure, though said components may not be labeled in or explicitly discussed with reference to FIGS. 51A-63. For example, the light therapy device mounting kit 1000 may include at least one elongate pole 120, bottom base 580, mounting device 200, brace 880, etc. For the sake of clarity, FIGS. 51A-63 include the reference labels most pertinent to the discussion of the mounting kit 1000, including the vertical spacer(s) 1018, square spacer(s) 1020, horizontal spacer(s) 1040, 0-ring(s) 1048, and cavities 1014, 1032, 1036, 1050, and 1054. In addition, the light therapy devices 1010, 1016, 1034, 1038, 1052, and 1056 may be substantially similar to the light therapy devices shown in previous figures and discussed earlier in this disclosure.

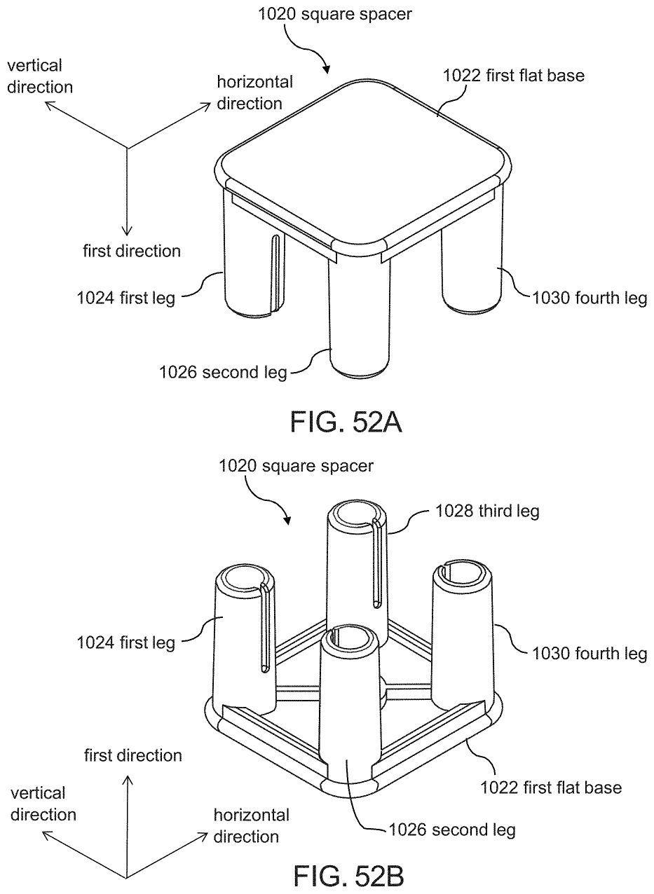

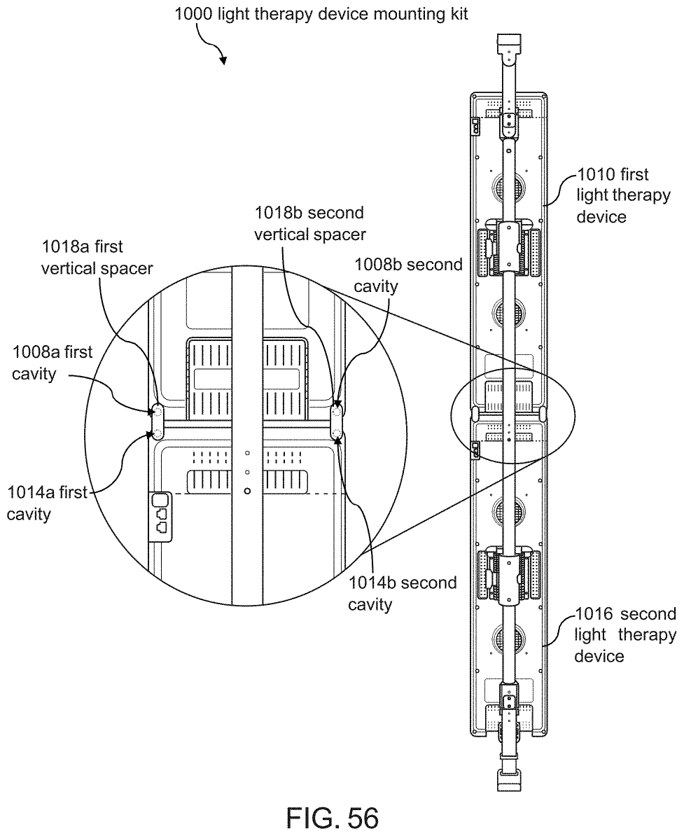

FIGS. 51A and 51B illustrate that, in some embodiments, a first spacer 1002 comprises a first flat base 1004 coupled to a first leg 1006 and a second leg 1012 spaced from the first leg 1006. The first leg 1006 and the second leg 10102 may be configured to protrude away from the first flat base 1004 along a first direction, as demonstrated by the directional indicators in FIGS. 51A and 51B. As will be discussed later in the disclosure, the first leg 1006 may be configured to slideably engage a first cavity 1008a located within a first light therapy device 1010 (see FIG. 56). Similarly, the second leg 1012 of the first spacer 1002 may be configured to slideably engage a first cavity 1014a located within a second light therapy device 1016 (see FIG. 56). In many embodiments, the first spacer 1002 defines a vertical spacer 1018. The first spacer 1002 may define a first vertical spacer 1018a. The first flat base 1004 may be elongate along a vertical direction perpendicular to the first direction, as indicated in FIGS. 51A and 51B. In some embodiments, the first leg 1006 of the vertical spacer 1018 is spaced from the second leg 1012 of the vertical spacer 1018 along the vertical direction. It should be noted that the perspective views shown in FIGS. 51A and 51B, as well as FIGS. 52A-B and 53A-B, may distort the directions such that they do not appear perpendicular.

FIGS. 52A and 52B illustrate top and bottom perspective views, respectively, of a square spacer 1020. In many embodiments, as shown, the square spacer 1020 comprises a first flat base 1022 elongate along a vertical direction and a horizontal direction perpendicular to the vertical direction, as well as a plurality of legs protruding away from the first flat base 1022 along the first direction, which may be perpendicular to the vertical and horizontal directions. The square spacer 1020 may include a first leg 1024, a second leg 1026, a third leg 1028, and a fourth leg 1030. Each of the first leg 1024, second leg 1026, third leg 1028, and fourth leg 1030 may be configured to slideably engage a cavity located within a light therapy device, thereby coupling up to four light therapy devices, as will be discussed later in the disclosure.

In many embodiments, the first leg 1024 of the square spacer 1020 is spaced from the second leg 1026 along the vertical direction and is spaced from the third leg 1028 along the horizontal direction. The second leg 1026 of the square spacer 1020 may be spaced from the first leg 1024 along the vertical direction, and may be spaced from the fourth leg 1030 along the horizontal direction. In some embodiments, the third leg 1028 of the square spacer 1020 is spaced from the first leg 1024 along the horizontal direction, and is spaced from the fourth leg 1030 along the vertical direction. The fourth leg 1030 of the square spacer 1020 may be spaced from the second leg 1026 along the horizontal direction, and may be spaced from the third leg 1028 along the vertical direction.

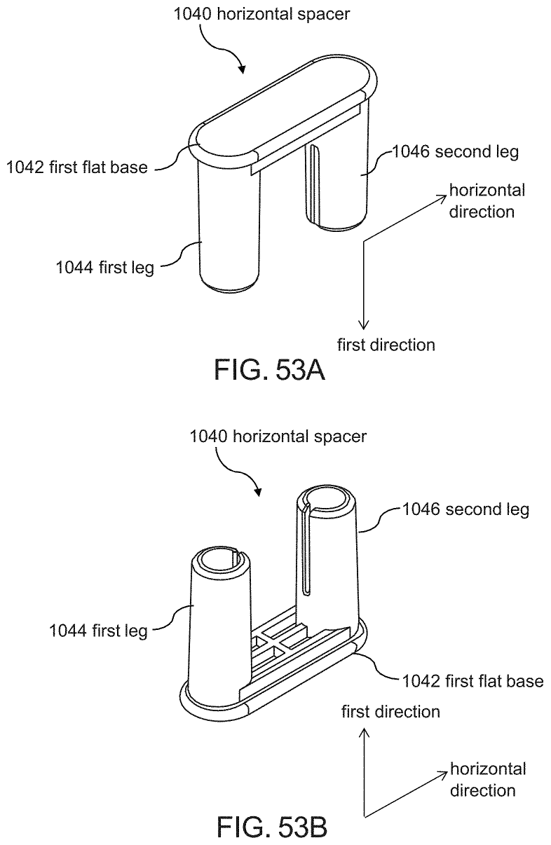

FIGS. 53A and 53B appear similar to FIGS. 51A and 51B, but illustrate perspective views of a horizontal spacer 1040. In some embodiments, the horizontal spacer 1040 includes a first flat base 1042 elongate along the horizontal direction, a first leg 1044 protruding away from the first flat base 1042 along the first direction, and a second leg 1046 protruding away from the first flat base 1042 along the first direction, which is perpendicular to the horizontal direction. The second leg 1046 may be spaced from the first leg 1044 along the horizontal direction. In many embodiments, the first leg 1044 and second leg 1046 are configured to slideably engage cavities located within light therapy devices, as will be discussed later in the disclosure. It should be noted that though the horizontal spacer 1040 and the vertical spacer 1018 may appear substantially the same, they may define different sizes. For example, in some embodiments, the distance between a center portion of the first leg 1006 and a center portion of the second leg 1012 of the vertical spacer 1018 is about 29 mm. The distance between a center portion of the first leg 1044 and a center portion of the second leg 1046 of the horizontal spacer 1040 may be about 27 mm. As such, in some embodiments, the vertical spacer 1018 is slightly larger than the horizontal spacer 1040. The size difference between the vertical spacer 1018 and the horizontal spacer 1040 may reflect that when multiple light therapy devices are coupled to one another, the devices may be closer when placed side-to-side as compared to when arranged top-to-bottom.

The spacing between the legs of the square spacer 1020 may correspond to both the vertical spacer 1018 and the horizontal spacer 1040. For example, the distance between a center portion of the first leg 1024 and a center portion of the second leg 1026, and/or between a center portion of the third leg 1028 and a center portion of the fourth leg 1030, of the square spacer 1020 may be about 29 mm, which corresponds to the size of the vertical spacer 1018. The distance between a center portion of the first leg 1024 and a center portion of the third leg 1028, and/or between a center portion of the second leg 1026 and a center portion of the fourth leg 1030, of the square spacer 1020 may be about 27 mm, which corresponds to the size of the horizontal spacer 1040. Accordingly, the “square” spacer 1020 may not define a perfect square shape. It should be noted that 27 mm and 29 mm are approximate measurements and intended to be non-limiting. Some embodiments of the vertical spacer 1018, square spacer 1020, and/or horizontal spacer 1040 may define different measurements than those listed here. In some embodiments, the horizontal spacer 1040 is slightly larger than the vertical spacer 1018. The horizontal spacer 1040 and the vertical spacer 1018 may be substantially the same size.

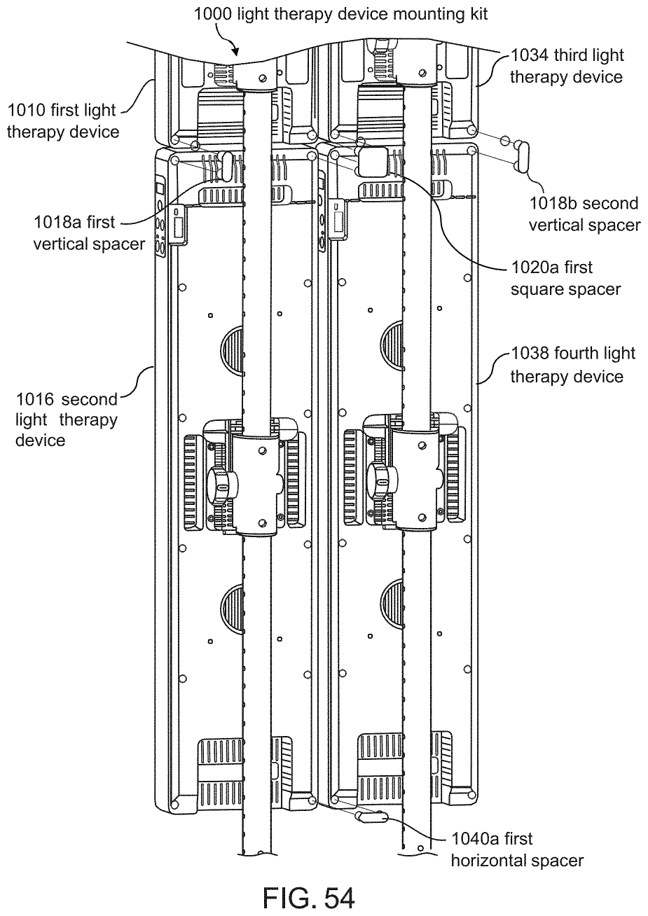

FIG. 54 shows a back perspective view of a light therapy device mounting kit 1000, including a first vertical spacer 1018a, a second vertical spacer 1018b, a first square spacer 1020a, and a first horizontal spacer 1040a. FIG. 54 also includes a first light therapy device 1010, a second light therapy device 1016, a third light therapy device 1034, and a fourth light therapy device 1038. In many embodiments, as illustrated in FIG. 54, the light therapy devices 1010, 1016, 1034, 1038 are coupled to one another via at least one vertical spacer 1018, at least one square spacer 1020, and at least one horizontal spacer 1040. Different combinations of light therapy devices and spacers are shown in, and will be discussed in greater detail with reference to, FIGS. 56-63.

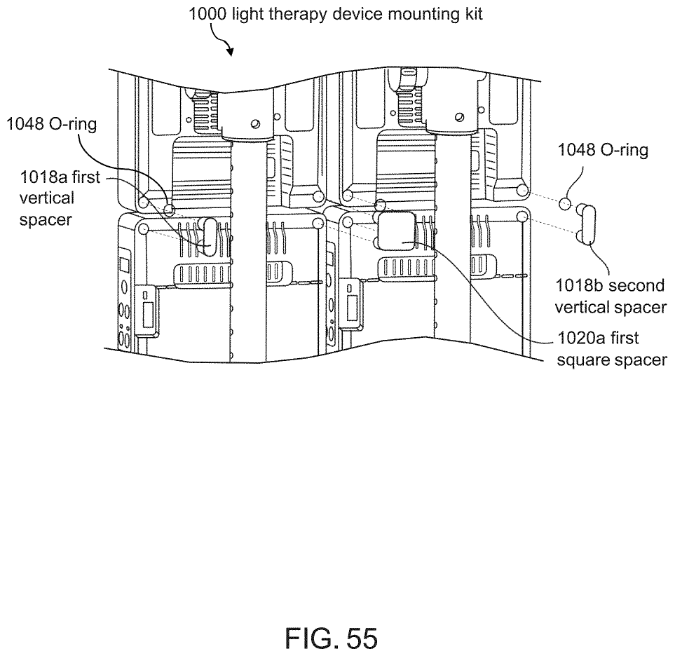

FIG. 55 shows a close-up view of the light therapy device mounting kit 1000 shown in FIG. 54, including the first vertical spacer 1018a, second vertical spacer 1018b, and first square spacer 1020a. FIG. 55 also includes two O-rings 1048. In some embodiments, the mounting kit 1000 includes at least one O-ring 1048, which is arranged and configured to slideably couple to at least one leg of at least one spacer. As shown in FIG. 55, each O-ring 1048 may be configured to couple to the first leg 1006 of the first vertical spacer 1018a and the first leg 1006 of the second vertical spacer 1018b. As previously mentioned, each leg of each of the spacers 1018, 1020, 1040 is configured to slideably engage a cavity located within each light therapy device 1010, 1016, 1034, 1038. In some embodiments, the cavities define different sizes in different light therapy devices, while the legs of each spacer 1018, 1020, 1040 define substantially the same size. For example, cavities of the first light therapy device 1010 and third light therapy device 1034 may define a larger size than cavities of the second light therapy device 1016 and fourth light therapy device 1038, and each O-ring 1048 may be used to ensure a snug and proper fit between each spacer 1018, 1020, 1040 and each light therapy device 1010, 1016, 1034, 1038. Though not shown in FIG. 55, in some embodiments, the mounting kit 1000 includes two additional O-rings 1048 configured to slideably couple to the first leg 1024 and third leg 1028 of the first square spacer 1020a, as those legs 1024, 1028 are configured to couple to the first and third light therapy devices 1010, 1034.

In some embodiments, each of the first leg 1006 of the vertical spacer 1018, the second leg 1012 of the vertical spacer 1018, the first leg 1024 of the square spacer 1020, the second leg 1026 of the square spacer 1020, the third leg 1028 of the square spacer 1020, the fourth leg 1030 of the square spacer 1020, the first leg 1044 of the horizontal spacer 1040, and the second leg 1046 of the horizontal spacer 1040 defines substantially the same length. The length may define about 25 mm from below each first flat base 1004, 1022, 1042, or about 31mm from the top of each first flat base 1004, 1022, 1042. Each of the recited legs of each spacer may also define substantially the same diameter. In some embodiments, the diameter is about 10 mm. Similar to previously listed dimensions, measurements of 25 mm, 31 mm, and 10 mm are used as examples and intended to be non-limiting dimensions. As previously mentioned, due to the equal-sized nature of each leg of each spacer, the O-ring 1048 may be used to compensate for differences in cavity sizes between light therapy devices 1010, 1016, 1034, 1038. The use of O-rings 1048 may help standardize each mounting kit 1000 by allowing for standard spacer sizes, rather than compensating for different sized light therapy devices by making different sized spacers. In some embodiments, each mounting kit 1000 includes a plurality of O-rings 1048.

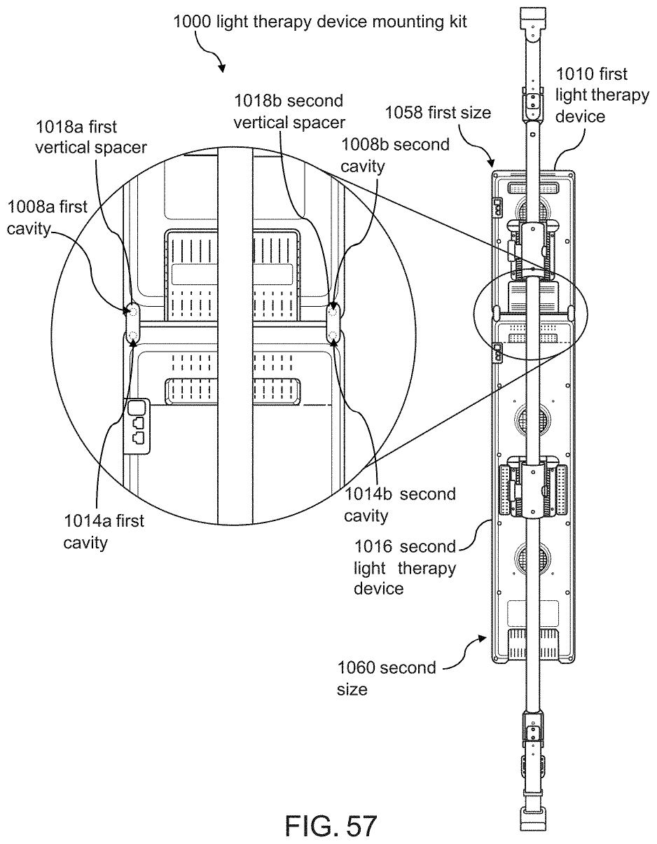

Referring now to FIG. 56, a back view and an inset view of the mounting kit 1000 coupled to a first light therapy device 1010 and a second light therapy device 1016 are shown. The inset view of FIG. 56 illustrates, in greater detail, the first vertical spacer 1018a and second vertical spacer 1018b coupled to the first and second light therapy devices 1010, 1016. Like the first vertical spacer 1018a, the second vertical spacer 1018b may comprise a first flat base 1004, a first leg 1006 protruding away from the first flat base 1004, and a second leg 1012 spaced from the first leg 1006 and also protruding away from the first flat base 1004. As shown in FIG. 56, the first vertical spacer 1018a may be configured to slideably engage a first cavity 1008a located in the first light therapy device 1010 and a first cavity 1014a located in the second light therapy device 1016. The second vertical spacer 1018b may be configured to slideably engage a second cavity 1008b located in the first light therapy device 1010 and a second cavity 1014b located in the second light therapy device 1016. In many embodiments, the first leg 1006 of each of the first vertical spacer 1018a and the second vertical spacer 1018b is configured to engage the cavity 1008 located in the first light therapy device 1010, and the second leg 1012 of each of the first vertical spacer 1018a and the second vertical spacer 1018b is configured to engage the cavity 1014 located in the second light therapy device 1016. Like the cavities shown in FIGS. 57-64, the cavities 1008a, 1008b, 1014a, 1014b are shown in dashed lines in FIG. 56 to indicate that when the first and second vertical spacers 1018a, 1018b are slideably coupled to each light therapy device 1010, 1016, the cavities 1008a, 1008b, 1014a, 1014b may not be visible.

In some embodiments, as illustrated in FIG. 57, the first light therapy device 1010 defines a first size 1058 and the second light therapy device 1016 defines a second size 1060, where the first size 1058 is smaller than the second size 1060. In such embodiments, the mounting kit 1000 may include a first O-ring 1048 and a second O-ring 1048, as shown in FIG. 55, wherein the first O-ring 1048 is configured to couple within the first cavity 1008a of the first light therapy device 1010, and the second O-ring 1048 is configured to couple within the second cavity 1008b of the first light therapy device 1010. As discussed with reference to FIG. 55, the cavities 1008a, 1008b of the first light therapy device 1010 may be a different size, for example have a larger diameter, than the cavities 1014a, 1014b of the second light therapy device 1016. In order to ensure a proper fit of the vertical spacers 1018a, 1018b within the cavities 1008, 1014 of both light therapy devices 1010, 1016, O-rings 1048 may be used. For example, each O-ring 1048 may be slideably coupled on the first leg 1006 of each of the first vertical spacer 1018a and the second vertical spacer 1018b prior to slideably coupling each spacer 1018a, 1018b to the first light therapy device 1010, in order to compensate for the extra space in each cavity 1008a, 1008b, as compared to the cavities 1014a, 1014b. Other than the use of O-rings 1048, the mounting kit 1000 illustrated in FIG. 57 may be substantially the same as the mounting kit 1000 illustrated in FIG. 56. In many embodiments, the mounting kit 1000 of FIGS. 56 and 57 comprises two vertical spacers 1018a, 1018b and at least two O-rings 1048. The mounting kit 1000 may include an extra third O-ring 1048.

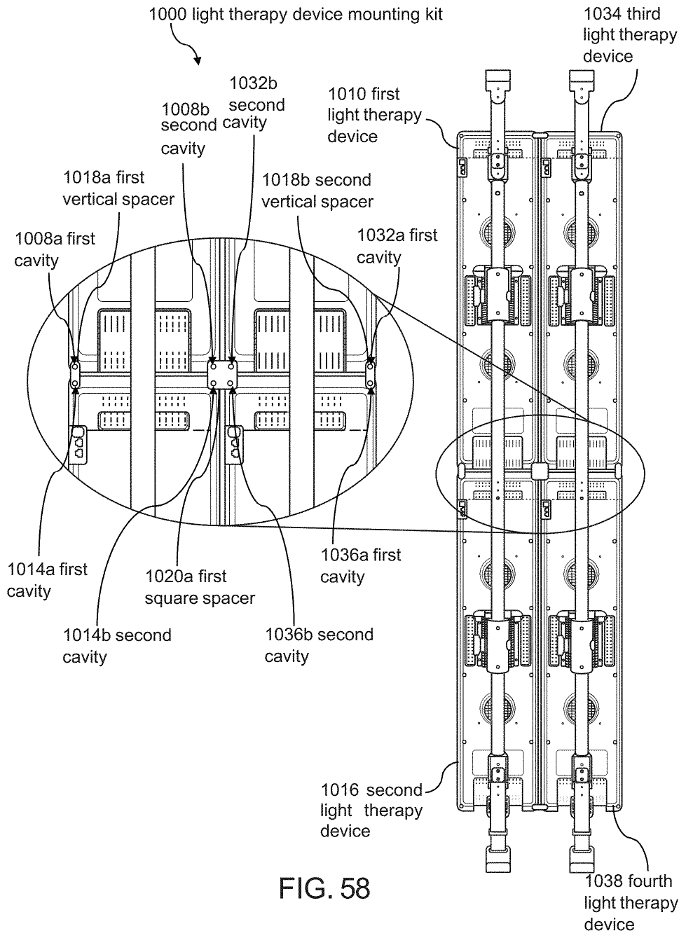

FIG. 58 shows another embodiment of the mounting kit 1000 and illustrates the kit 1000 coupling together a first light therapy device 1010, a second light therapy device 1016, a third light therapy device 1034, and a fourth light therapy device 1038. FIG. 58 also includes an inset view of the junction of the four light therapy devices 1010, 1016, 1034, 1038, which shows a first vertical spacer 1018a, a second vertical spacer 1018b, and a first square spacer 1020a. In many embodiments, and as also shown in FIGS. 54-57, the first vertical spacer 1018a is configured to couple the first light therapy device 1010 and the second light therapy device 1016 by slideably engaging a first cavity 1008a located within the first light therapy device 1010 and a first cavity 1014a located within the second light therapy device 1016. Similarly, the second vertical spacer 1018b may be configured to couple the third light therapy device 1034 and the fourth light therapy device 1038 by slideably engaging a first cavity 1032a located within the third light therapy device 1034 and a first cavity 1036a located within the fourth light therapy device 1038. In many embodiments, the first leg 1006 of the first vertical spacer 1018a is configured to be received by the first cavity 1008a of the first light therapy device 1010 and the second leg 1012 of the first vertical spacer 1018a is configured to be received by the first cavity 1014a of the second light therapy device 1016. The first leg 1006 of the second vertical spacer 1018b may be configured to be received by the first cavity 1032a of the third light therapy device 1034, and the second leg 1012 of the second vertical spacer 1018b may be configured to be received by the first cavity 1036a of the fourth light therapy device 1038.

The inset view of FIG. 58 also includes the first square spacer 1020a. In many embodiments, the first square spacer 1020a is configured to couple all four light therapy devices 1010, 1016, 1034, 1038 by slideably engaging a second cavity 1008b, 1014b, 1032b, 1036b located within each respective device 1010, 1016, 1034, 1038. The first leg 1024 of the first square spacer 1020a may be configured to be received by the second cavity 1008b of the first light therapy device 1010, and the second leg 1026 of the first square spacer 1020a may be configured to be received by the second cavity 1014b of the second light therapy device 1016. In some embodiments, the third leg 1028 of the first square spacer 1020a is configured to be received by the second cavity 1032b of the third light therapy device 1034, and the fourth leg 1030 of the first square spacer 1020a is configured to be received by the second cavity 1036b of the fourth light therapy device 1038.

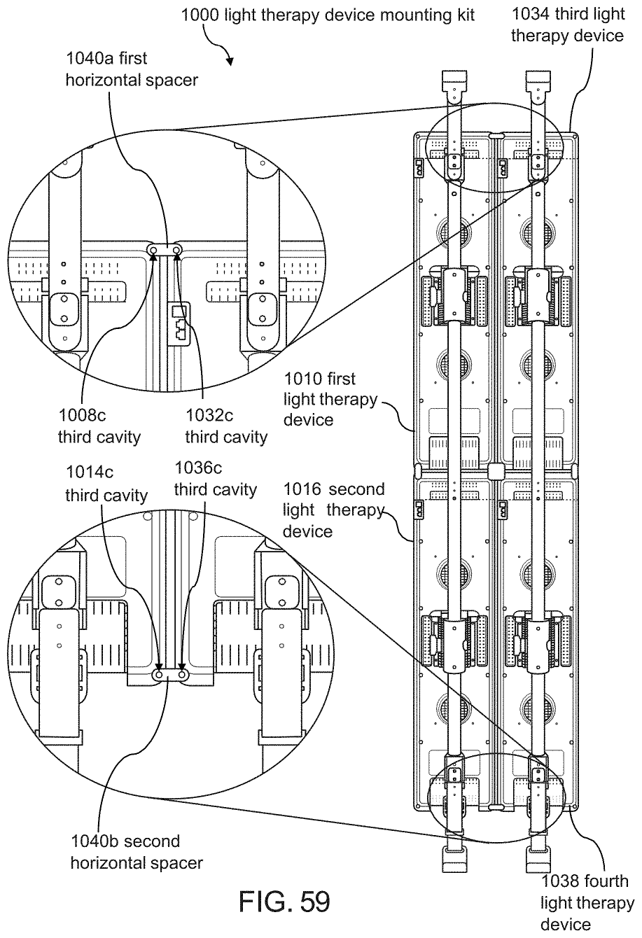

FIG. 59 shows the same mounting kit 1000 of FIG. 58, but includes inset views of the first and second horizontal spacers 1040a, 1040b. In some embodiments, as shown in FIG. 59, the first horizontal spacer 1040a is configured to couple the first light therapy device 1010 and the third light therapy device 1034, and the second horizontal spacer 1040b is configured to couple the second light therapy device 1016 and the fourth light therapy device 1038. The first horizontal spacer 1040a may include a first leg 1044 configured to slideably engage a third cavity 1008c of the first light therapy device 1010 and a second leg 1046 configured to slideably engage a third cavity 1032c of the third light therapy device 1034. Similarly, the second horizontal spacer 1040b may include a first leg 1044 configured to slideably engage a third cavity 1014c of the second light therapy device 1016 and a second leg 1046 configured to slideably engage a third cavity 1036c of the fourth light therapy device 1038.

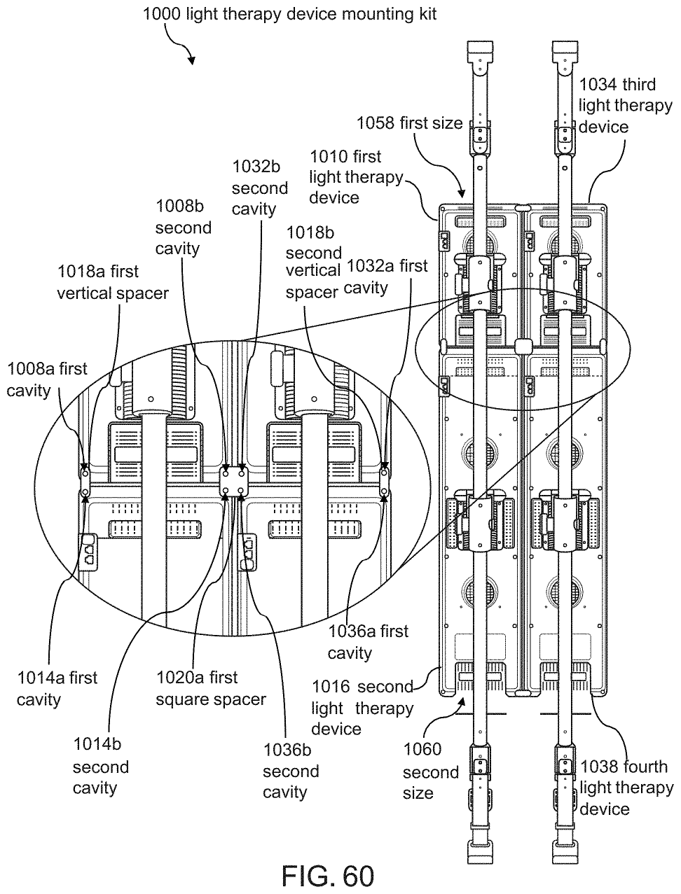

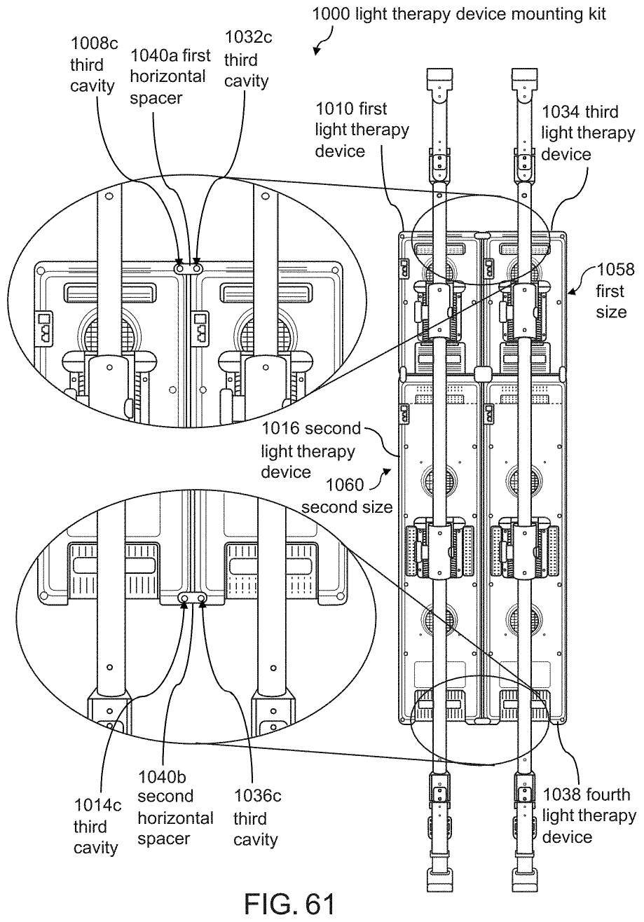

FIG. 60 is similar to FIG. 58, but shows an embodiment where the first light therapy device 1010 and the third light therapy device 1034 define a first size 1058 while the second light therapy device 1016 and the fourth light therapy device 1038 define a second size 1060. As previously discussed, when the light therapy devices 1010, 1016, 1034, 1038 define different sizes, at least one O-ring 1048 may be used to facilitate secure coupling of at least one of the spacers 1018, 1020, 1040 to the smaller light therapy devices. For example, in the embodiment shown in FIG. 60, 0-rings 1048 may be coupled to each of the first leg 1006 of the first vertical spacer 1018a, the first leg 1006 of the second vertical spacer 1018b, the first leg 1024 of the first square spacer 1020a, and the third leg 1028 of the first square spacer 1020a. In addition, O-rings 1048 may be coupled to both the first leg 1044 and second leg 1046 of the first horizontal spacer 1040a shown in FIG. 61. Stated differently, O-rings 1048 may be configured to couple to each leg of each spacer 1018a, 1018b, 1020a, 1040a that slideably couples to a light therapy device of the first size 1058, such as the first light therapy device 1010 in FIG. 57 and the first and third light therapy devices 1010, 1034 in FIGS. 60 and 61.

Similar to FIG. 59, FIG. 61 shows the mounting kit 1000 including inset views of the first and second horizontal spacers 1040a, 1040b. In many embodiments, the first horizontal spacer 1040a is configured to couple to a third cavity 1008c of the first light therapy device 1010 and a third cavity 1032c of the third light therapy device 1034. The second horizontal spacer 1040b may be configured to couple to a third cavity 1014c of the second light therapy device 1016 and a third cavity 1036c of the fourth light therapy device 1038. As previously stated, each of the first leg 1044 and second leg 1046 of the first horizontal spacer 1040a may be configured to couple to an O-ring 1048 prior to coupling to, respectively, the third cavity 1008c of the first light therapy device 1010 and the third cavity 1032c of the third light therapy device 1034. Due to the fact that the second and fourth light therapy devices 1016, 1038 may define the second size 1060, the first leg 1044 and second leg 1046 of the second horizontal spacer 1040b may not need to couple to O-rings 1048 prior to slideably engaging the third cavity 1014c of the second light therapy device 1016 and the third cavity 1036c of the fourth light therapy device 1038, respectively. In some embodiments, the mounting kit 1000 shown in FIGS. 58-61 comprises two vertical spacers 1018a, 1018b, one square spacer 1020a, two horizontal spacers 1040a, 1040b, and at least six O-rings 1048. The mounting kit 1000 may include an additional seventh O-ring 1048.

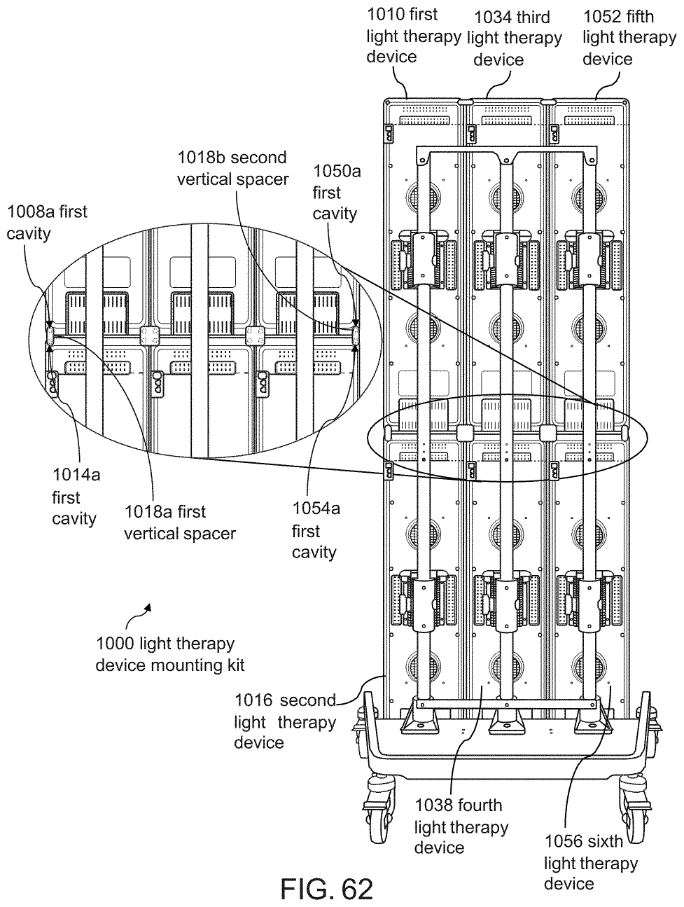

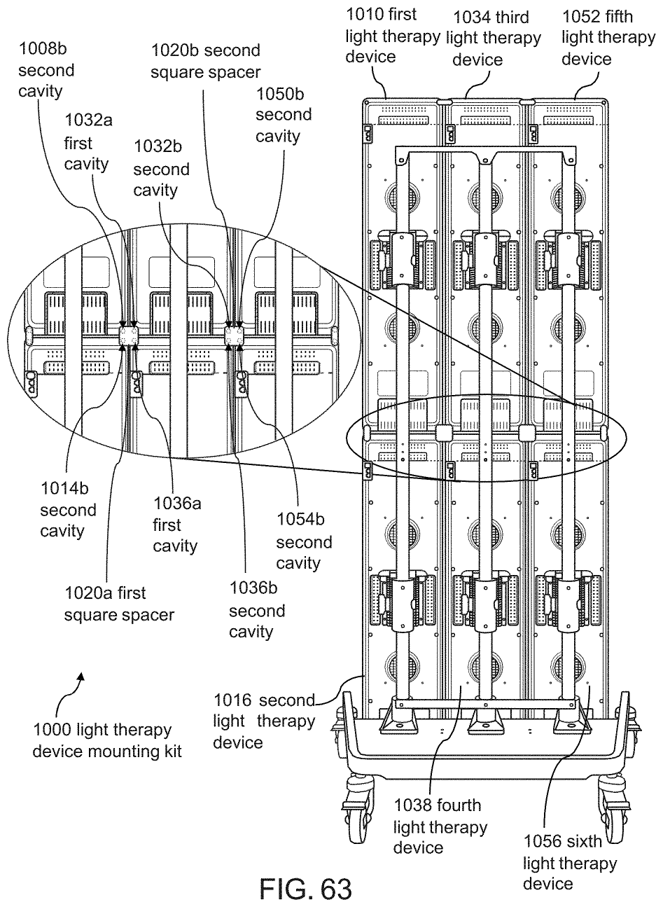

FIGS. 62-64 illustrate embodiments of the mounting kit 1000 coupling together a first light therapy device 1010, a second light therapy device 1016, a third light therapy device 1034, a fourth light therapy device 1038, a fifth light therapy device 1052, and a sixth light therapy device 1056. FIGS. 62 and 63 both include inset views showing the first vertical spacer 1018a, the second vertical spacer 1018b, the first square spacer 1020a, and the second square spacer 1020b. It should be noted that FIGS. 62 and 63 are substantially the same drawing but include different reference labels, for the sake of clarity of the Figures.

FIG. 62 includes the first vertical spacer 1018a, which may be configured to couple the first and second light therapy devices 1010, 1016, and the second vertical spacer 1018b, which may be configured to couple the fifth and sixth light therapy devices 1052, 1056. As discussed with reference to earlier Figures, in many embodiments, the first leg 1006 of the first vertical spacer 1018a is configured to slideably engage a first cavity 1008a of the first light therapy device 1010, and the second leg 1012 of the first vertical spacer 1018a is configured to slideably engage a first cavity 1014a of the second light therapy device 1016. A first leg 1006 of the second vertical spacer 1018b may be configured to slideably engage a first cavity 1050a of the fifth light therapy device 1052, and a second leg 1012 of the second vertical spacer 1018b may be configured to slideably engage a first cavity 1054a of the sixth light therapy device 1056.

FIG. 63 illustrates the first square spacer 1020a and the second square spacer 1020b of the mounting kit 1000. As shown, the first square spacer 1020a may be configured to couple the first, second, third, and fourth light therapy devices 1010, 1016, 1034, 1038, and the second square spacer 1020b may be configured to couple the third, fourth, fifth, and sixth light therapy devices 1034, 1038, 1052, 1056. In some embodiments, a first leg 1024 of the first square spacer 1020a is configured to slideably couple to a second cavity 1008b of the first light therapy device 1010 and a second leg 1026 of the first square spacer 1020a is configured to slideably couple to a second cavity 1014b of the second light therapy device 1016. A third leg 1028 of the first square spacer 1020a may be configured to slideably engage a first cavity 1032a of the third light therapy device 1034, and a fourth leg 1030 of the first square spacer 1020a may be configured to slideably engage a first cavity 1036a of the fourth light therapy device 1038.

In some embodiments, a first leg 1024 of the second square spacer 1020b is configured to slideably engage a second cavity 1032b of the third light therapy device 1034, and a second leg 1026 of the second square spacer 1020b is configured to slideably engage a second cavity 1036b of the fourth light therapy device 1038. A third leg 1028 of the second square spacer 1020b may be configured to slideably couple to a second cavity 1050b of the fifth light therapy device 1052, and a fourth leg 1030 of the second square spacer may be configured to slideably couple to a second cavity 1054b of the sixth light therapy device 1056.

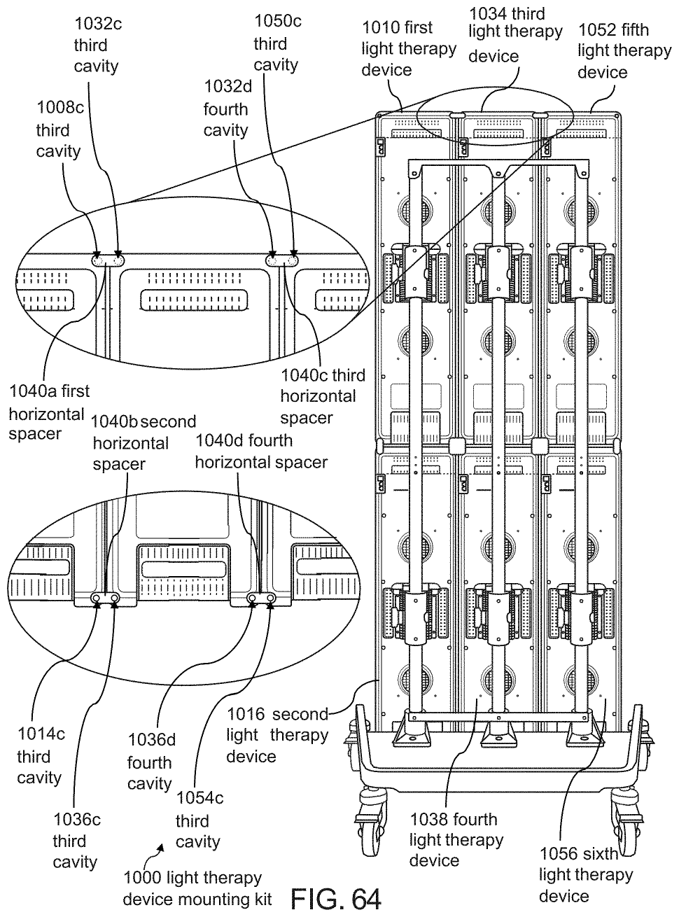

FIG. 64 shows inset views of first, second, third, and fourth horizontal spacers 1040a, 1040b, 1040c, 1040d. In some embodiments, the first horizontal spacer 1040a is configured to couple the first and third light therapy devices 1010, 1034, the second horizontal spacer 1040b is configured to couple the second and fourth light therapy devices 1016, 1038, the third horizontal spacer 1040c is configured to couple the third and fifth light therapy devices 1034, 1052, and the fourth horizontal spacer 1040d is configured to couple the fourth and sixth light therapy devices 1038, 1056. A first leg 1044 of the first horizontal spacer 1040a may be configured to slideably couple to a third cavity 1008c of the first light therapy device 1010, and a second leg 1046 of the first horizontal spacer 1040a may be configured to slideably couple to a third cavity 1032c of the third light therapy device 1034. A first leg 1044 of the third horizontal spacer 1040c may be configured to slideably couple to a fourth cavity 1032d of the third light therapy device 1034, and a second leg 1046 of the third horizontal spacer 1040c may be configured to slideably couple to a third cavity 1050c of the fifth light therapy device 1052.

Turning now to the lower inset view of FIG. 64, an illustration of the second and fourth horizontal spacers 1040b, 1040d is shown. It should be noted that the larger, zoomed-out image of FIG. 64 does not include these horizontal spacers 1040b, 1040d, as they are obscured by the mobile stand shown coupled to the light therapy devices 1010, 1016, 1034, 1038, 1052, 1056. In some embodiments, a first leg 1044 of the second horizontal spacer 1040b is configured to slideably engage a third cavity 1014c of the second light therapy device 1016, and a second leg 1046 of the second horizontal spacer 1040b is configured to slideably engage a third cavity 1036c of the fourth light therapy device 1038. A first leg 1044 of the fourth horizontal spacer 1040d may be configured to slideably couple to a fourth cavity 1036d of the fourth light therapy device 1038, and a second leg 1046 of the fourth horizontal spacer 1040d may be configured to slideably couple to a third cavity 1054c of the sixth light therapy device 1056.

It should be noted that as each of the first, second, third, fourth, fifth, and sixth light therapy devices 1010, 1016, 1034, 1038, 1052, and 1056 is shown in FIGS. 62-64 as the same size, there may not be a need to include O-rings 1048 in the mounting kit 1000 of FIGS. 62-64. In some embodiments, at least one of the light therapy devices 1010, 1016, 1034, 1038, 1052, 1056 defines the first size 1058 illustrated in FIGS. 57, 60, and 61, and is configured to be coupled to at least one O-ring 1048 as discussed with reference to FIGS. 57, 60, and 61. The mounting kit 1000 of FIGS. 62-64 may comprise two vertical spacers 1018a, 1018b, two square spacers 1020a, 1020b, and four horizontal spacers 1040a, 1040b, 1040c, 1040d.

In many embodiments, each of the cavities 1008, 1014, 1032, 1036, 1050, 1054 is located adjacent a corner of each light therapy device 1010, 1016, 1034, 1038, 1052, 1056. Each cavity 1008, 1014, 1032, 1036, 1050, 1054 may define a depth substantially equal to or greater than the length of each leg of each spacer 1018, 1020, 1040; for example, equal to or greater than about 25mm. When each spacer 1018, 1020, 1040 is coupled to each light therapy device 1010, 1016, 1034, 1038, 1052, 1056, each first flat base 1004, 1022, 1042 may be configured to protrude about 6 mm from a back surface of each light therapy device 1010, 1016, 1034, 1038, 1052, 1056. In some embodiments, each leg of each spacer 1018, 1020, 1040 defines a substantially equal and constant diameter throughout the length of each leg. Each leg may be tapered and define a greater diameter adjacent the first flat base 1004, 1022, 1042 than at each distal end of each leg. In some embodiments, the inverse is true and each leg defines a smaller diameter adjacent the first flat base 1004, 1022, 1042.

As shown in FIGS. 51A-B, 52A-B, and 53A-B, each leg may comprise a non-continuous surface, including a split located on each leg running along the first direction. The split may allow for a degree of compression of the diameter of each leg. For example, the split may enable the diameter of a leg to decrease, which may be helpful in coupling a spacer 1018, 1020, 1040 to a cavity of variable/reduced size (as compared to a standard cavity size). Stated differently, the split may enable a slight “squeezing” of the leg (e.g., reducing the width of the split) in order to fit the leg inside the narrower cavity. The split may also be beneficial from a manufacturing standpoint, particularly in an embodiment where each spacer 1018, 1020, 1040 is made from an injection mold. In many embodiments, each leg of each spacer 1018, 1020, 1040 is hollow. The hollow nature of each leg may help reduce weight of the overall mounting kit 1000.

Each spacer 1018, 1020, 1040 may be configured to slideably couple with each cavity via a friction fit. Stated differently, coupling and decoupling each spacer 1018, 1020, 1040 to each light therapy device 1010, 1016, 1034, 1038, 1052, 1056 may comprise a simple push and pull motion, respectively, similar to “plugging” and “unplugging” a power cord from an electrical socket. The light therapy device mounting kit 1000 may be configured for use in conjunction with at least one bracket, such as the at least one brace 880 shown coupling each elongate pole 120 in FIG. 42 (FIGS. 62-64 also show, though do not label, at least one brace 880). In some embodiments, the mounting kit 1000 is configured to be used in place of at least one bracket, as illustrated in FIGS. 58-61. Each spacer 1018, 1020, 1040 may be configured to keep the light therapy devices 1010, 1016, 1034, 1038, 1052, 1056 aligned and immobile in each desired configuration, such as the configurations shown in FIGS. 56-64.

The following dimensions are included as examples only, and are intended to be nonlimiting. Any of the following dimensions may be greater or lesser than the actual dimensions of each spacer/each element of each spacer. Accordingly, in some embodiments, the spacers define the dimensions shown below.

Vertical Spacer 1018

| | | |
|---|---|---|
| Flat base length | 43.2 | mm |
| Flat base width | 14.5 | mm |
| Flat base depth | 6.0 | mm |
| Leg length (below base) | 25.0 | mm |
| Distance between legs (center portion) | 28.7 | mm |
| Distal leg diameter | 10.3 | mm |
| Proximal leg diameter | 11.25 | mm |
| Leg split length | 18.0 | mm |
| Leg split width | 1.25 | mm |

Square Spacer 1020

| | | |
|---|---|---|
| Flat base length | 43.2 | mm |
| Flat base width | 41.25 | mm |
| Flat base depth | 6.0 | mm |
| Leg length (below base) | 25.0 | mm |
| Distance between 1^{st} and 2^{nd}/3^{rd} | 28.7 | mm |
| and 4^{th} legs (center portion) | | |
| Distance between 1^{st} and 3^{rd}/2^{nd} | 26.8 | mm |
| and 4^{th} legs (center portion) | | |
| Distal leg diameter | 10.4 | mm |
| Proximal leg diameter | 11.25 | mm |
| Leg split length | 18.0 | mm |
| Leg split width | 1.25 | mm |

Horizontal Spacer 1040

| | | |
|---|---|---|
| Flat base height | 14.5 | mm |
| Flat base width | 41.25 | mm |
| Flat base depth | 6.0 | mm |
| Leg length (below base) | 25.0 | mm |
| Distance between legs (center portion) | 26.8 | mm |
| Distal leg diameter | 10.3 | mm |
| Proximal leg diameter | 11.25 | mm |
| Leg split length | 18 | mm |
| Leg split width | 1.25 | mm |

### Materials

Any of the components disclosed herein, including but not limited to, the elongate pole 120, the mounting device 200, the top hook 240a, the bottom hook 240b, the light therapy device bracket 320, the at least one projection 370, the hollow cylinder 380, the mounting device bracket 400, the clip 420, the knob 460, the protrusion 480, the top cap 520a, the bottom cap 520b, the extension bar 540, the bottom base 580, the mobile stand 600, the plurality of wheels 620, the at least one coupling mechanism 700, the fixed stand 720, the wall bracket 760, the plurality of feet 820, the C-shaped hook 840, the at least one brace 880, the light therapy device floor stand 900, the floor plate 920, the first curved side arm 940a, the second curved side arm 940b, the light therapy device table stand 104, the base 110, the arm 112, and/or back bracket 114 may comprise any suitable material or combination of materials.

For example, the components may comprise any metallic, plastic, or combination material. Metallic materials may comprise steel, aluminum, or any other metal or combination of metals. In many embodiments, the components are comprised of durable material(s) configured to withstand everyday wear-and-tear and more significant potential damage, such as being dropped, bumped, knocked against a wall and/or door, etc. The components may comprise any color, whether the natural color of a material (e.g., metallic components may be silver in color) or an applied color (e.g., paint, powder coating, etc.).

Each spacer 1002, 1018, 1020, 1040 may comprise ABS plastic. In some embodiments, each spacer 1002, 1018, 1020, 1040 comprises glass fiber filled nylon with a 30% glass fiber fill. Similar to other listed components, the spacers 1002, 1018, 1020, 1040 may include an applied color (e.g., paint, powder coating, etc.), pattern, design, logo, etc. Any pattern, design, logo, or the like may be applied via etching or a similar process, or may comprise applied color (e.g., paint, powder coating, etc.). Each spacer 1002, 1018, 1020, 1040 may comprise a metal material, or a combination of metal and plastic materials. Each O-ring 1048 may comprise plastic, rubber, or a similar material.

### Interpretation

None of the steps described herein is essential or indispensable. Any of the steps can be adjusted or modified. Other or additional steps can be used. Any portion of any of the steps, processes, structures, and/or devices disclosed or illustrated in one embodiment, flowchart, or example in this specification can be combined or used with or instead of any other portion of any of the steps, processes, structures, and/or devices disclosed or illustrated in a different embodiment, flowchart, or example. The embodiments and examples provided herein are not intended to be discrete and separate from each other.

The section headings and subheadings provided herein are nonlimiting. The section headings and subheadings do not represent or limit the full scope of the embodiments described in the sections to which the headings and subheadings pertain. For example, a section titled “Topic 1” may include embodiments that do not pertain to Topic 1 and embodiments described in other sections may apply to and be combined with embodiments described within the “Topic 1” section. Some of the devices, systems, embodiments, and processes use computers. Each of the routines, processes, methods, and algorithms described in the preceding sections may be embodied in, and fully or partially automated by, code modules executed by one or more computers, computer processors, or machines configured to execute computer instructions. The code modules may be stored on any type of non-transitory computer-readable storage medium or tangible computer storage device, such as hard drives, solid state memory, flash memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method, event, state, or process blocks may be omitted in some implementations. The methods, steps, and processes described herein are also not limited to any particular sequence, and the blocks, steps, or states relating thereto can be performed in other sequences that are appropriate. For example, described tasks or events may be performed in an order other than the order specifically disclosed. Multiple steps may be combined in a single block or state. The example tasks or events may be performed in serial, in parallel, or in some other manner. Tasks or events may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present.

The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments can include A, B, and C. The term “and/or” is used to avoid unnecessary redundancy.

The term “about” is used to mean “approximately.” For example, the disclosure includes, “the distance between a center portion of the first leg 1006 and a center portion of the second leg 1012 of the vertical spacer 1018 is about 29 mm.” In this context, “about 29 mm” means “approximately 29 mm.” Any distance between 28 mm and 30 mm may fall into a suitable range of “about 29 mm,” as used in this disclosure.

The term “substantially” is used to mean “completely/exactly” or “nearly completely/exactly.” For example, the disclosure includes, “Each of the recited legs of each spacer may also define substantially the same diameter.” As shown in the tables above, the distal diameter of the legs of the vertical and horizontal spacers defines 10.3 mm, while the distal diameter of the legs of the square spacer defines 10.4 mm. As understood in this disclosure, 10.3 mm and 10.4 mm may be considered “substantially” the same, as they are “nearly exactly” the same.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein.

What is claimed is:

1. A light therapy device mounting kit, comprising:
a first spacer including:
a first flat base of the first spacer;
a first leg of the first spacer protruding away from the first flat base along a first direction, the first leg of the first spacer arranged and configured to slideably engage a first cavity located within a first light therapy device; and
a second leg of the first spacer protruding away from the first flat base along the first direction, the second leg of the first spacer spaced from the first leg of the first spacer, the second leg of the first spacer arranged and configured to slideably engage a first cavity located within a second light therapy device.

2. The light therapy device mounting kit of claim 1, wherein the first flat base is elongate along a vertical direction perpendicular to the first direction, and wherein the first spacer defines a first vertical spacer.

3. The light therapy device mounting kit of claim 2, wherein the first leg of the first vertical spacer is spaced from the second leg of the first vertical spacer along the vertical direction.

4. The light therapy device mounting kit of claim 2, further comprising:
a first square spacer including:
a first flat base of the first square spacer elongate along the vertical direction and a horizontal direction perpendicular to the first direction and the vertical direction;
a first leg of the first square spacer protruding away from the first flat base of the first square spacer along the first direction, the first leg of the first square spacer arranged and configured to slideably engage a second cavity located within the first light therapy device;
a second leg of the first square spacer protruding away from the first flat base of the first square spacer along the first direction, the second leg of the first square spacer spaced from the first leg of the first square spacer, the second leg of the first square spacer arranged and configured to slideably engage a second cavity located within the second light therapy device;
a third leg of the first square spacer protruding away from the first flat base of the first square spacer along the first direction, the third leg of the first square spacer spaced from the first leg of the first square spacer and the second leg of the first square spacer, the third leg of the first square spacer arranged and configured to slideably engage a first cavity located within a third light therapy device; and
a fourth leg protruding away from the first flat base of the first square spacer along the first direction, the fourth leg of the first square spacer spaced from the first leg of the first square spacer, the second leg of the first square spacer, and the third leg of the first square spacer, the fourth leg of the first square spacer arranged and configured to slideably engage a first cavity located within a fourth light therapy device.

5. The light therapy device mounting kit of claim 4, wherein the first leg of the first square spacer is spaced from the second leg of the first square spacer along the vertical direction, and spaced from the third leg of the first square spacer along the horizontal direction, wherein second leg of the first square spacer is spaced from the first leg of the first square spacer along the vertical direction, and spaced from the fourth leg of the first square spacer along the horizontal direction, wherein the third leg of the first square spacer is spaced from the first leg of the first square spacer along the horizontal direction, and spaced from the fourth leg of the first square spacer along the vertical direction, and wherein the fourth leg of the first square spacer is spaced from the second leg of the first square spacer along the horizontal direction, and spaced from the third leg of the first square spacer along the vertical direction.

6. The light therapy device mounting kit of claim 4, further comprising:
a first horizontal spacer including:
a first flat base of the first horizontal spacer elongate along the horizontal direction;
a first leg of the first horizontal spacer protruding away from the first flat base of the first horizontal spacer along the first direction, the first leg of the first horizontal spacer arranged and configured to slideably engage a third cavity located within the second light therapy device; and
a second leg of the first horizontal spacer protruding away from the first flat base of the first horizontal spacer along the first direction, the second leg of the first horizontal spacer spaced from the first leg of the first horizontal spacer, the second leg of the first horizontal spacer arranged and configured to slideably engage a second cavity within the fourth light therapy device.

7. The light therapy device mounting kit of claim 6, wherein the first leg of the first horizontal spacer is spaced from the second leg of the first horizontal spacer along the horizontal direction.

8. The light therapy device mounting kit of claim 2, further comprising a first O-ring arranged and configured to slideably couple to one of the first leg of the first vertical spacer and the second leg of the first vertical spacer.

9. The light therapy device mounting kit of claim 8, further comprising:
a second vertical spacer including:
a first flat base of the second vertical spacer elongate along the vertical direction;
a first leg of the second vertical spacer protruding away from the first flat base along the first direction, the first leg of the second vertical spacer arranged and configured to slideably engage a first cavity located within a third light therapy device; and
a second leg of the second vertical spacer protruding away from the first flat base along the first direction, the second leg of the second vertical spacer spaced from the first leg of the second vertical spacer, the second leg of the second vertical spacer arranged and configured to slideably engage a first cavity located within a fourth light therapy device.

10. The light therapy device mounting kit of claim 9, further comprising a second O-ring and a third O-ring, wherein each of the second O-ring and the third O-ring is arranged and configured to slideably couple to at least one leg of the first vertical spacer and at least one leg of the second vertical spacer.

11. The light therapy device mounting kit of claim 10, further comprising:
a first square spacer including:
a first flat base of the first square spacer elongate along the vertical direction and a horizontal direction perpendicular to the first direction and the vertical direction;
a first leg of the first square spacer protruding away from the first flat base of the first square spacer along the first direction, the first leg of the first square spacer arranged and configured to slideably engage a second cavity located within the first light therapy device;
a second leg of the first square spacer protruding away from the first flat base of the first square spacer along the first direction, the second leg of the first square spacer spaced from the first leg of the first square spacer, the second leg of the first square spacer arranged and configured to slideably engage a second cavity located within the second light therapy device;
a third leg of the first square spacer protruding away from the first flat base of the first square spacer along the first direction, the third leg of the first square spacer spaced from the first leg of the first square spacer and the second leg of the first square spacer, the third leg of the first square spacer arranged and configured to slideably engage a second cavity located within the third light therapy device; and
a fourth leg protruding away from the first flat base of the first square spacer along the first direction, the fourth leg of the first square spacer spaced from the first leg of the first square spacer, the second leg of the first square spacer, and the third leg of the first square spacer, the fourth leg of the first square spacer arranged and configured to slideably engage a second cavity located within the fourth light therapy device;
a first horizontal spacer including:
a first flat base of the first horizontal spacer elongate along the horizontal direction;
a first leg of the first horizontal spacer protruding away from the first flat base of the first horizontal spacer along the first direction, the first leg of the first horizontal spacer arranged and configured to slideably engage a third cavity located within the first light therapy device; and
a second leg of the first horizontal spacer protruding away from the first flat base of the first horizontal spacer along the first direction, the second leg of the first horizontal spacer spaced from the first leg of the first horizontal spacer, the second leg of the first horizontal spacer arranged and configured to slideably engage a third cavity within the third light therapy device; and
a second horizontal spacer including:
a first flat base of the second horizontal spacer elongate along the horizontal direction;
a first leg of the second horizontal spacer protruding away from the first flat base of the second horizontal spacer along the first direction, the first leg of the second horizontal spacer arranged and configured to slideably engage a third cavity located within the second light therapy device; and
a second leg of the second horizontal spacer protruding away from the first flat base of the second horizontal spacer along the first direction, the second leg of the second horizontal spacer spaced from the first leg of the second horizontal spacer, the second leg of the second horizontal spacer arranged and configured to slideably engage a third cavity within the fourth light therapy device.

12. The light therapy device mounting kit of claim 11, further comprising:
a fourth O-ring, a fifth O-ring, a sixth O-ring, and a seventh O-ring, wherein each of the fourth O-ring, the fifth O-ring, the sixth O-ring, and the seventh O-ring is arranged and configured to slideably couple to at least one leg of the first square spacer, at least one leg of the first horizontal spacer, and at least one leg of the second horizontal spacer.

13. The light therapy device mounting kit of claim 6, further comprising:
a second vertical spacer including:
a first flat base of the second vertical spacer elongate along the vertical direction;
a first leg of the second vertical spacer protruding away from the first flat base along the first direction, the first leg of the second vertical spacer arranged and configured to slideably engage a first cavity located within a fifth light therapy device; and
a second leg of the second vertical spacer protruding away from the first flat base along the first direction, the second leg of the second vertical spacer spaced from the first leg of the second vertical spacer, the second leg of the second vertical spacer arranged and configured to slideably engage a first cavity located within a sixth light therapy device.

14. The light therapy device mounting kit of claim 13, further comprising:
a second square spacer including:
a first flat base of the first square spacer elongate along the vertical direction and a horizontal direction perpendicular to the first direction and the vertical direction;
a first leg of the first square spacer protruding away from the first flat base of the first square spacer along the first direction, the first leg of the first square spacer arranged and configured to slideably engage a second cavity located within the third light therapy device;
a second leg of the first square spacer protruding away from the first flat base of the first square spacer along the first direction, the second leg of the first square spacer spaced from the first leg of the first square spacer, the second leg of the first square spacer arranged and configured to slideably engage a third cavity located within the fourth light therapy device;
a third leg of the first square spacer protruding away from the first flat base of the first square spacer along the first direction, the third leg of the first square spacer spaced from the first leg of the first square spacer and the second leg of the first square spacer, the third leg of the first square spacer arranged and configured to slideably engage a second cavity located within the fifth light therapy device; and
a fourth leg protruding away from the first flat base of the first square spacer along the first direction, the fourth leg of the first square spacer spaced from the first leg of the first square spacer, the second leg of the first square spacer, and the third leg of the first square spacer, the fourth leg of the first square spacer arranged and configured to slideably engage a second cavity located within the sixth light therapy device.

15. The light therapy device mounting kit of claim 14, further comprising:
a second horizontal spacer including:
a first flat base of the second horizontal spacer elongate along the horizontal direction;
a first leg of the second horizontal spacer protruding away from the first flat base of the second horizontal spacer along the first direction, the first leg of the second horizontal spacer arranged and configured to slideably engage a third cavity located within the first light therapy device; and
a second leg of the first horizontal spacer protruding away from the first flat base of the first horizontal spacer along the first direction, the second leg of the first horizontal spacer spaced from the first leg of the first horizontal spacer, the second leg of the first horizontal spacer arranged and configured to slideably engage a third cavity within the third light therapy device;
a third horizontal spacer including:
a first flat base of the third horizontal spacer elongate along the horizontal direction;
a first leg of the third horizontal spacer protruding away from the first flat base of the third horizontal spacer along the first direction, the first leg of the third horizontal spacer arranged and configured to slideably engage a third cavity located within the fourth light therapy device; and
a second leg of the third horizontal spacer protruding away from the first flat base of the third horizontal spacer along the first direction, the second leg of the third horizontal spacer spaced from the first leg of the third horizontal spacer, the second leg of the third horizontal spacer arranged and configured to slideably engage a third cavity within the sixth light therapy device; and
a fourth horizontal spacer including:
a first flat base of the fourth horizontal spacer elongate along the horizontal direction;
a first leg of the fourth horizontal spacer protruding away from the first flat base of the fourth horizontal spacer along the first direction, the first leg of the fourth horizontal spacer arranged and configured to slideably engage a fourth cavity located within the third light therapy device; and
a second leg of the fourth horizontal spacer protruding away from the first flat base of the fourth horizontal spacer along the first direction, the second leg of the fourth horizontal spacer spaced from the first leg of the fourth horizontal spacer, the second leg of the fourth horizontal spacer arranged and configured to slideably engage a third cavity within the fifth light therapy device.

16. A light therapy system, comprising:
a first light therapy device comprising a first front treatment surface and a first back surface located opposite the first front treatment surface;
a second light therapy device comprising a second front treatment surface and a second back surface located opposite the second front treatment surface;
a first vertical spacer including:
a first flat base of the first vertical spacer elongate along a vertical direction;
a first leg of the first vertical spacer protruding away from the first flat base along
a first direction that is perpendicular to the vertical direction, the first leg of the first vertical spacer arranged and configured to slideably engage a first cavity located within the first light therapy device; and
a second leg of the first vertical spacer protruding away from the first flat base along the first direction, the second leg of the first vertical spacer spaced from the first leg of the first vertical spacer, the second leg of the first vertical spacer arranged and configured to slideably engage a first cavity located within a second light therapy device; and
a second vertical spacer including:
a first flat base of the second vertical spacer elongate along the vertical direction;
a first leg of the second vertical spacer protruding away from the first flat base along the first direction, the first leg of the second vertical spacer arranged and configured to slideably engage a second cavity located within the first light therapy device; and
a second leg of the second vertical spacer protruding away from the first flat base along the first direction, the second leg of the second vertical spacer spaced from the first leg of the second vertical spacer, the second leg of the second vertical spacer arranged and configured to slideably engage a second cavity located within the second light therapy device.

17. The light therapy system of claim 16, wherein the first light therapy device defines a first size and the second light therapy device defines a second size, wherein the first size is smaller than the second size, the system further comprising:
a first O-ring arranged and configured to slideably couple to the first leg of the first vertical spacer, such that the first O-ring is arranged and configured to couple within the first cavity of the first light therapy device; and
a second O-ring arranged and configured to slideably couple to the first leg of the second vertical spacer, such that the second O-ring is arranged and configured to couple within the second cavity of the first light therapy device.

18. The light therapy system of claim 16, further comprising:
a third light therapy device comprising a third front treatment surface and a third back surface located opposite the third front treatment surface, wherein the first leg of the second vertical spacer is arranged and configured to slideably engage a first cavity located within the third light therapy device;
a fourth light therapy device comprising a fourth front treatment surface and a fourth back surface located opposite the fourth front treatment surface, wherein the second leg of the second vertical spacer is arranged and configured to slideably engage a first cavity located within the fourth light therapy device;
a first square spacer including:
a first flat base of the first square spacer elongate along the vertical direction and a horizontal direction perpendicular to the first direction and the vertical direction;
a first leg of the first square spacer protruding away from the first flat base of the first square spacer along the first direction, the first leg of the first square spacer arranged and configured to slideably engage a second cavity located within the first light therapy device;
a second leg of the first square spacer protruding away from the first flat base of the first square spacer along the first direction, the second leg of the first square spacer spaced from the first leg of the first square spacer, the second leg of the first square spacer arranged and configured to slideably engage a second cavity located within the second light therapy device;
a third leg of the first square spacer protruding away from the first flat base of the first square spacer along the first direction, the third leg of the first square spacer spaced from the first leg of the first square spacer and the second leg of the first square spacer, the third leg of the first square spacer arranged and configured to slideably engage a second cavity located within the third light therapy device; and
a fourth leg protruding away from the first flat base of the first square spacer along the first direction, the fourth leg of the first square spacer spaced from the first leg of the first square spacer, the second leg of the first square spacer, and the third leg of the first square spacer, the fourth leg of the first square spacer arranged and configured to slideably engage a second cavity located within the fourth light therapy device;
a first horizontal spacer including:
a first flat base of the first horizontal spacer elongate along the horizontal direction;
a first leg of the first horizontal spacer protruding away from the first flat base of the first horizontal spacer along the first direction, the first leg of the first horizontal spacer arranged and configured to slideably engage a third cavity located within the first light therapy device; and
a second leg of the first horizontal spacer protruding away from the first flat base of the first horizontal spacer along the first direction, the second leg of the first horizontal spacer spaced from the first leg of the first horizontal spacer, the second leg of the first horizontal spacer arranged and configured to slideably engage a third cavity within the third light therapy device; and
a second horizontal spacer including:
a first flat base of the second horizontal spacer elongate along the horizontal direction;
a first leg of the second horizontal spacer protruding away from the first flat base of the second horizontal spacer along the first direction, the first leg of the second horizontal spacer arranged and configured to slideably engage a third cavity located within the second light therapy device; and
a second leg of the second horizontal spacer protruding away from the first flat base of the second horizontal spacer along the first direction, the second leg of the second horizontal spacer spaced from the first leg of the second horizontal spacer, the second leg of the second horizontal spacer arranged and configured to slideably engage a third cavity within the fourth light therapy device.

19. The light therapy system of claim 18, wherein the first light therapy device defines a first size, the second light therapy device defines a second size, the third light therapy device defines a third size, and the fourth light therapy device defines a fourth size, wherein the first size and third size are smaller than the second size and fourth size, the system further comprising:
a first O-ring arranged and configured to slideably couple to the first leg of the first vertical spacer, such that the first O-ring is arranged and configured to couple within the first cavity of the first light therapy device;
a second O-ring arranged and configured to slideably couple to the first leg of the second vertical spacer, such that the second O-ring is arranged and configured to couple within the first cavity of the third light therapy device;
a third O-ring arranged and configured to slideably couple to the first leg of the first square spacer, such that the third O-ring is arranged and configured to couple within the second cavity of the first light therapy device;
a fourth O-ring arranged and configured to slideably couple to the third leg of the first square spacer, such that the fourth O-ring is arranged and configured to couple within the second cavity of the third light therapy device;
a fifth O-ring arranged and configured to slideably couple to the first leg of the first horizontal spacer, such that the fifth O-ring is arranged and configured to couple within the third cavity of the first light therapy device; and
a sixth O-ring arranged and configured to slideably couple to the second leg of the first horizontal spacer, such that the sixth O-ring is arranged and configured to couple within the third cavity of the third light therapy device.

20. The light therapy system of claim 18, further comprising:
a fifth light therapy device comprising a fifth front treatment surface and a fifth back surface located opposite the fifth front treatment surface, wherein the first leg of the second vertical spacer is arranged and configured to slideably engage a first cavity located within the fifth light therapy device;
a sixth light therapy device comprising a sixth front treatment surface and a sixth back surface located opposite the sixth front treatment surface, wherein the second leg of the second vertical spacer is arranged and configured to slideably engage a first cavity located within the sixth light therapy device;
the first square spacer, wherein the first leg of the first square spacer is arranged and configured to slideably engage a second cavity located within the first light therapy device, the second leg of the first square spacer is arranged and configured to slideably engage a second cavity located within the second light therapy device, the third leg of the first square spacer is arranged and configured to slideably engage a first cavity located within the third light therapy device, and the fourth leg of the first square spacer is arranged and configured to slideably engage a first cavity located within the fourth light therapy device;
a second square spacer including:
a first flat base of the second square spacer elongate along the vertical direction and a horizontal direction perpendicular to the first direction and the vertical direction;
a first leg of the second square spacer protruding away from the first flat base of the second square spacer along the first direction, the first leg of the second square spacer arranged and configured to slideably engage a second cavity located within the third light therapy device;
a second leg of the second square spacer protruding away from the first flat base of the second square spacer along the first direction, the second leg of the second square spacer spaced from the first leg of the second square spacer, the second leg of the second square spacer arranged and configured to slideably engage a second cavity located within the fourth light therapy device;
a third leg of the second square spacer protruding away from the first flat base of the second square spacer along the first direction, the third leg of the second square spacer spaced from the first leg of the second square spacer and the second leg of the second square spacer, the third leg of the second square spacer arranged and configured to slideably engage a second cavity located within the fifth light therapy device; and
a fourth leg protruding away from the first flat base of the second square spacer along the first direction, the fourth leg of the second square spacer spaced from the first leg of the second square spacer, the second leg of the second square spacer, and the third leg of the second square spacer, the fourth leg of the second square spacer arranged and configured to slideably engage a second cavity located within the sixth light therapy device;
a third horizontal spacer including:
a first flat base of the third horizontal spacer elongate along the horizontal direction;
a first leg of the third horizontal spacer protruding away from the first flat base of the third horizontal spacer along the first direction, the first leg of the third horizontal spacer arranged and configured to slideably engage a fourth cavity located within the third light therapy device; and
a second leg of the third horizontal spacer protruding away from the first flat base of the third horizontal spacer along the first direction, the second leg of the third horizontal spacer spaced from the first leg of the third horizontal spacer, the second leg of the third horizontal spacer arranged and configured to slideably engage a third cavity within the fifth light therapy device; and
a fourth horizontal spacer including:
a first flat base of the fourth horizontal spacer elongate along the horizontal direction;
a first leg of the fourth horizontal spacer protruding away from the first flat base of the fourth horizontal spacer along the first direction, the first leg of the fourth horizontal spacer arranged and configured to slideably engage a fourth cavity located within the fourth light therapy device; and
a second leg of the fourth horizontal spacer protruding away from the first flat base of the fourth horizontal spacer along the first direction, the second leg of the fourth horizontal spacer spaced from the first leg of the fourth horizontal spacer, the second leg of the fourth horizontal spacer arranged and configured to slideably engage a third cavity within the sixth light therapy device.
